SEQUENCE LISTING (9 sequences)

SEQ ID NO: 1 - DNA, 2893 bp, Homo sapiens
ggcaccagctcagccccaagccactgctctcccatcccagtccctggaaatccacccacttggcccagctcaccccaactccaacccactgggacccagtctccaggggcctgactgtgggcggcagccactcctgagtgagcaaaggttcctccgcggtgctctcccgtccagagccctgctgatggggaagtccgaaggccccgtggggatggtggagagcgctggccgtgcagggcagaagcgcccggggttcctggagggggggctgctgctgctgctgctgctggtgaccgctgccctggtggccttgggtgtcctctacgccgaccgcagagggaagcagctgccacgccttgctagccggctgtgcttcttacaggaggagaggacctttgtaaaacgaaaaccccgagggatcccagaggcccaagaggtgagcgaggtctgcaccacccctggctgcgtgatagcagctgccaggatcctccagaacatggacccgaccacggaaccgtgtgacgacttctaccagtttgcatgcggaggctggctgcggcgccacgtgatccctgagaccaactcaagatacagcatctttgacgtcctccgcgacgagctggaggtcatcctcaaagcggtgctggagaattcgactgccaaggaccggccggctgtggagaaggccaggacgctgtaccgctcctgcatgaaccagagtgtgatagagaagcgaggctctcagcccctgctggacatcttggaggtggtgggaggctggccggtggcgatggacaggtggaacgagaccgtaggactcgagtgggagctggagcggcagctggcgctgatgaactcacagttcaacaggcgcgtcctcatcgacctcttcatctggaacgacgaccagaactccagccggcacatcatctacatagaccagcccaccttgggcatgccctcccgagagtactacttcaacggcggcagcaaccggaaggtgcgggaagcctacctgcagttcatggtgtcagtggccacgttgctgcgggaggatgcaaacctgcccagggacagctgcctggtgcaggaggacatggtgcaggtgctggagctggagacacagctggccaaggccacggtaccccaggaggagagacacgacgtcatcgccttgtaccaccggatgggactggaggagctgcaaagccagtttggcctgaagggatttaactggactctgttcatacaaactgtgctatcctctgtcaaaatcaagctgctgccagatgaggaagtggtggtctatggcatcccctacctgcagaaccttgaaaacatcatcgacacctactcagccaggaccatacagaactacctggtctggcgcctggtgctggaccgcattggtagcctaagccagagattcaaggacacacgagtgaactaccgcaaggcgctgtttggcacaatggtggaggaggtgcgctggcgtgaatgtgtgggctacgtcaacagcaacatggagaacgccgtgggctccctctacgtcagggaggcgttccctggagacagcaagagcatggtcagagaactcattgacaaggtgcggacagtgtttgtggagacgctggacgagctgggctggatggacgaggagtccaagaagaaggcgcaggagaaggccatgagcatccgggagcagatcgggcaccctgactacatcctggaggagatgaacaggcgcctggacgaggagtactccaatctgaacttctcagaggacctgtactttgagaacagtctgcagaacctcaaggtgggcgcccagcggagcctcaggaagcttcgggaaaaggtggacccaaatctctggatcatcggggcggcggtggtcaatgcgttctactccccaaaccgaaaccagattgtattccctgccgggatcctccagccccccttcttcagcaaggagcagccacaggccttgaactttggaggcattgggatggtgatcgggcacgagatcacgcacggctttgacgacaatggccggaacttcgacaagaatggcaacatgatggattggtggagtaacttctccacccagcacttccgggagcagtcagagtgcatgatctaccagtacggcaactactcctgggacctggcagacgaacagaacgtgaacggattcaacacccttggggaaaacattgctgacaacggaggggtgcggcaagcctataaggcctacctcaagtggatggcagagggtggcaaggaccagcagctgcccggcctggatctcacccatgagcagctcttcttcatcaactatgcccaggtgtggtgcgggtcctaccggcccgagttcgccatccaatccatcaagacagacgtccacagtcccctgaagtacagggtactggggtcgctgcagaacctggccgccttcgcagacacgttccactgtgcccggggcacccccatgcaccccaaggagcgatgccgcgtgtggtagccaaggccctgccgcgctgtgcggcccacgcccacctgctgctcggaggcatctgtgcgaaggtgcagctagcggcgacccagtgtacgtcccgccccggccaaccatgccaagcctgcctgccaggcctctgcgcctggcctagggtgcagccacctgcctgacacccagggatgagcagtgtccagtgcagtacctggaccggagccccctccacagacacccgcggggctcagtgcccccgtcacagctctgtagagacaatcaactgtgtcctgcccaccctccaaggtgcattgtcttccagtatctacagcttcagacttgagctaagtaaatgcttcaaagaaaaaaaaaaaaaaaaaaaaa

SEQ ID NO: 2 - protein, 779 aa, Homo sapiens
MGKSEGPVGMVESAGRAGQKRPGFLEGGLLLLLLLVTAALVALGVLYADRRGKQLPRLASRLCFLQEERTFVKRKPRGIPEAQEVSEVCTTPGCVIAAARILQNMDPTTEPCDDFYQFACGGWLRRHVIPETNSRYSIFDVLRDELEVILKAVLENSTAKDRPAVEKARTLYRSCMNQSVIEKRGSQPLLDILEVVGGWPVAMDRWNETVGLEWELERQLALMNSQFNRRVLIDLFIWNDDQNSSRHIIYIDQPTLGMPSREYYFNGGSNRKVREAYLQFMVSVATLLREDANLPRDSCLVQEDMVQVLELETQLAKATVPQEERHDVIALYHRMGLEELQSQFGLKGFNWTLFIQTVLSSVKIKLLPDEEVVVYGIPYLQNLENIIDTYSARTIQNYLVWRLVLDRIGSLSQRFKDTRVNYRKALFGTMVEEVRWRECVGYVNSNMENAVGSLYVREAFPGDSKSMVRELIDKVRTVFVETLDELGWMDEESKKKAQEKAMSIREQIGHPDYILEEMNRRLDEEYSNLNFSEDLYFENSLQNLKVGAQRSLRKLREKVDPNLWIIGAAVVNAFYSPNRNQIVFPAGILQPPFFSKEQPQALNFGGIGMVIGHEITHGFDDNGRNFDKNGNMMDWWSNFSTQHFREQSECMIYQYGNYSWDLADEQNVNGFNTLGENIADNGGVRQAYKAYLKWMAEGGKDQQLPGLDLTHEQLFFINYAQVWCGSYRPEFAIQSIKTDVHSPLKYRVLGSLQNLAAFADTFHCARGTPMHPKERCRVW

SEQ ID NO: 3 - DNA, 20 bp, Homo sapiens
ctgtcttgatggattggatg

SEQ ID NO: 4 - DNA, 22 bp, Homo sapiens
gtccttggcagtcgaattctcc

SEQ ID NO: 5 - DNA, 2975 bp, Homo sapiens
cagagctcgtttagtgaaccgtcagaattttgtaatacgactcactatagggcggccgcgaattcggcaccagctcagccccaagccactgctctcccatcccagtccctggaaatccacccacttggcccagctcaccccaactccaacccactgggacccagtctccaggggcctgactgtgggcggcagccactcctgagtgagcaaaggttcctccgcggtgctctcccgtccagagccctgctgatggggaagtccgaaggccccgtggggatggtggagagcgctggccgtgcagggcagaagcgcccggggttcctggagggggggctgctgctgctgctgctgctggtgaccgctgccctggtggccttgggtgtcctctacgccgaccgcagagggaagcagctgccacgccttgctagccggctgtgcttcttacaggaggagaggacctttgtaaaacgaaaaccccgagggatcccagaggcccaagaggtgagcgaggtctgcaccacccctggctgcgtgatagcagctgccaggatcctccagaacatggacccgaccacggaaccgtgtgacgacttctaccagtttgcatgcggaggctggctgcggcgccacgtgatccctgagaccaactcaagatacagcatctttgacgtcctccgcgacgagctggaggtcatcctcaaagcggtgctggagaattcgactgccaaggaccggccggctgtggagaaggccaggacgctgtaccgctcctgcatgaaccagagtgtgatagagaagcgaggctctcagcccctgctggacatcttggaggtggtgggaggctggccggtggcgatggacaggtggaacgagaccgtaggactcgagtgggagctggagcggcagctggcgctgatgaactcacagttcaacaggcgcgtcctcatcgacctcttcatctggaacgacgaccagaactccagccggcacatcatctacatagaccagcccaccttgggcatgccctcccgagagtactacttcaacggcggcagcaaccggaaggtgcgggaagcctacctgcagttcatggtgtcagtggccacgttgctgcgggaggatgcaaacctgcccagggacagctgcctggtgcaggaggacatggtgcaggtgctggagctggagacacagctggccaaggccacggtaccccaggaggagagacacgacgtcatcgccttgtaccaccggatgggactggaggagctgcaaagccagtttggcctgaagggatttaactggactctgttcatacaaactgtgctatcctctgtcaaaatcaagctgctgccagatgaggaagtggtggtctatggcatcccctacctgcagaaccttgaaaacatcatcgacacctactcagccaggaccatacagaactacctggtctggcgcctggtgctggaccgcattggtagcctaagccagagattcaaggacacacgagtgaactaccgcaaggcgctgtttggcacaatggtggaggaggtgcgctggcgtgaatgtgtgggctacgtcaacagcaacatggagaacgccgtgggctccctctacgtcagggaggcgttccctggagacagcaagagcatggtcagagaactcattgacaaggtgcggacagtgtttgtggagacgctggacgagctgggctggatggacgaggagtccaagaagaaggcgcaggagaaggccatgagcatccgggagcagatcgggcaccctgactacatcctggaggagatgaacaggcgcctggacgaggagtactccaatctgaacttctcagaggacctgtactttgagaacagtctgcagaacctcaaggtgggcgcccagcggagcctcaggaagcttcgggaaaaggtggacccaaatctctggatcatcggggcggcggtggtcaatgcgttctactccccaaaccgaaaccagattgtattccctgccgggatcctccagccccccttcttcagcaaggagcagccacaggccttgaactttggaggcattgggatggtgatcgggcacgagatcacgcacggctttgacgacaatggccggaacttcgacaagaatggcaacatgatggattggtggagtaacttctccacccagcacttccgggagcagtcagagtgcatgatctaccagtacggcaactactcctgggacctggcagacgaacagaacgtgaacggattcaacacccttggggaaaacattgctgacaacggaggggtgcggcaagcctataaggcctacctcaagtggatggcagagggtggcaaggaccagcagctgcccggcctggatctcacccatgagcagctcttcttcatcaactatgcccaggtgtggtgcgggtcctaccggcccgagttcgccatccaatccatcaagacagacgtccacagtcccctgaagtacagggtactggggtcgctgcagaacctggccgccttcgcagacacgttccactgtgcccggggcacccccatgcaccccaaggagcgatgccgcgtgtggtagccaaggccctgccgcgctgtgcggcccacgcccacctgctgctcggaggcatctgtgcgaaggtgcagctagcggcgacccagtgtacgtcccgccccggccaaccatgccaagcctgcctgccaggcctctgcgcctggcctagggtgcagccacctgcctgacacccagggatgagcagtgtccagtgcagtacctggaccggagccccctccacagacacccgcggggctcagtgcccccgtcacagctctgtagagacaatcaactgtgtcctgcccaccctccaaggtgcattgtcttccagtatctacagcttcagacttgagctaagtaaatgcttcaaagaaaaaaaaaaaaaaaaaaaaactcgactctagattgcg

SEQ ID NO: 6 - protein, 9 aa, Homo sapiens
MGKSEGPVG

SEQ ID NO: 7 - DNA, 27 bp, Homo sapiens
atggggaagtccgaaggccccgtgggg

SEQ ID NO: 8 - protein, 9 aa, Synthetic
GGFLRRVCA

SEQ ID NO: 9 - protein, 6 aa, Synthetic
GGFLRR

(19) United States
(12) Patent Application Publication
(10) Pub. No.: US 2004/0014131 A1
(43) Pub. Date: Jan. 22, 2004

(54) Assay methods

(75) Inventors: Neil Benson, Sandwich (GB); Helen Frances Boyd, Sandwich (GB); Peter Stacey, Sandwich (GB); Leonard Gabriel Contillo, Oakdale, CT (US); David Harlan Singleton, Noank, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/192,407

(22) Filed: Jul. 9, 2002

(51) Int. Cl. G01N 33/53; C12Q 1/37

(52) U.S. Cl. 435/7.1; 435/23

(57) ABSTRACT

The invention relates to methods for identifying agents, which bind to and/or modulate exopeptidases or endopeptidases, preferably human soluble secreted endopeptidase (SEP), oxytocinase, neutral endopeptidase (NEP), or non-human SEP, preferably comprising contacting said agents with a polypeptide comprising: SEQ ID NO: 2, an amino acid sequence encoded by the polynucleotide sequence of SEQ ID NO: 1 or SEQ ID NO: 5; or a polypeptide encoded by the cDNA of NCIMB 41110, and determining whether binding and/or modulation occurs. Preferably, said method is carried out in the presence of a substrate peptide labelled with at least one fluorescent donor dye and at least one fluorescence acceptor dye, with the method used to detect said agents being a competitive binding Fluorescence Resonance Energy Transfer (FRET) assay.

### ASSAY METHODS

This application claims priority under 35 U.S.C.§119(e), from U.S. provisional application 60/220,908 filed Jul. 26, 2000, and under 35 U.S.C.§120 from U.S. application Ser. Nos. 09/905,846 filed Jul. 13, 2001 and 09/948,429 filed Sep. 7, 2001; and under 35 U.S.C.§119(a) from G.B. application 0017387.2 filed Jul. 24, 2000. All of these applications are incorporated herein by reference.

### FIELD OF THE INVENTION

The present invention relates to methods for identifying agents, which bind to and/or modulate exopeptidases or endopeptidases, preferably human soluble secreted endopeptidase (SEP), oxytocinase, neutral endopeptidase (NEP), or non-human SEP.

Preferred methods are those which are carried out in the presence of a substrate peptide labelled with at least one fluorescent donor dye and at least one fluorescence acceptor dye, with the method used to detect said agents being a competitive binding Fluorescence Resonance Energy Transfer (FRET) assay.

The present invention also relates to agents (candidate modulators, preferably candidate inhibitors or selective inhibitors) identified by the methods of the invention.

### BACKGROUND OF THE INVENTION

### HUMAN SOLUBLE SECRETED ENDOPEPTIDASE (HUMAN SEP)

Proteases form a large family of enzymes that cleave proteins and peptides at the peptide bond that forms the backbone of the peptide or protein chain. Proteases are found in all organisms from bacteria to man. In humans, approximately 1% of all genes (400-1000) are predicted to encode protease enzymes. They participate in activation and maturation of nascent polypeptides, the degradation of mis-folded and damaged proteins, and the controlled turnover of peptides and proteins both inside, and outside, the cell. Their activities are important for many processes including digestion, normal growth, endocrine function, wound healing, inflammation, angiogenesis, tissue remodelling during embryonic development, tumour metastasis, cardiovascular disease, neurological disease, and bacterial, parasitic, and viral infections.

Proteases can be broadly categorised on the basis of where they cleave their substrate. Exopeptidases, which include aminopeptidases, dipeptidyl peptidases, tripeptidases, carboxypeptidases, peptidyl-di-peptidases, dipeptidases and omega peptidases, cleave residues at the termini of their substrate. Endopeptidases, including serine proteases, cysteine proteases, and metalloendopeptidases, cleave at a sequence within the peptide.

An important group of endopeptidases known as zinc metalloproteases are characterised by having a requirement for the binding of a zinc ion in their catalytic site. Zinc metalloproteases can be subdivided into classes (for review see FEBS Letters 354 (1994) pp. 1-6), with one such class being the neprilysin (NEP)-like zinc metalloproteases (FASEB Journal, Vol 11, 1997 pp. 355-384). The NEP class includes at least 7 enzymes that are structurally related to each other (see later). They are typically membrane-bound, with a large carboxy-terminal extracellular domain, a short membrane-spanning region, and a short intracellular domain at the amino terminus. Known members of this family are neprilysin (also called neutral endopeptidase (NEP), CD10, CALLA, enkephalinase or EC 3.4.24.11), endothelin-converting enzymes (ECE-1 and ECE-2), PEX, KELL, X-converting enzyme/damage induced neural endopeptidase (XCE/DINE), and an enzyme identified in rodents called soluble secreted endopeptidase/neprilysin II (SEP/NEPII; Ghaddar, G et al, Biochem Journal, Vol 347, 2000, pp. 419-429; Ikeda, K et al, Journal Biological Chemistry, Vol 274, 1999, pp. 32469-32477; Tanja, O et al, Biochem Biophys Research Communication, Vol 271, 2000, pp. 565-570; International Patent Application WO 99/53077). The functions of the members of this class are thought to be related to peptidergic signalling. This is a process that occurs in most organisms, including humans, in which peptide molecules are used as “messengers” to elicit physiological responses. This typically involves the production and release of the peptide messenger by a specific cell, sometimes as an inactive precursor that is cleaved by a protease to become active. The active form of the peptide then binds a specific receptor on the surface of another cell where it elicits a response. The peptide is then inactivated by degradation by another protease.

NEP was the first member of this class to be discovered. NEP is a promiscuous protease in that it is able to proteolyse and inactivate many biological peptides, e.g. enkephalins, bradykinin and substance P. It usually cleaves the peptide on the amino-terminal side of a hydrophobic residue. NEP can be found in many tissues of the body and is most abundant in kidney. Its physiological functions are not fully understood, but one indication from the phenotype of NEP “knockout” mice is that it is involved in preventing endotoxic shock.

ECE-1 is a protein 37% identical to NEP. ECE-1 is broadly distributed throughout the body and converts the inactive precursor peptide big-endothelin into endothelin, which is a potent vasoconstrictor, important for maintaining vascular tone. The ECE-2 enzyme is derived form a separate gene to ECE-1, but its amino acid sequence is similar, with an overall homology of 59%. The physiological importance of ECE-2 to endothelin production is unclear. ECE-2 mRNA is present at much lower levels then ECE-1 mRNA and it has a different pH optimum to ECE-1, being inactive at neutral pH, and most active at pH 5.5.

The KELL enzyme is a clinically important member of the NEP class found in erythroid tissues. The antigens of the KELL blood group system reside in this protein which can cause haemolytic reaction to blood transfusions.

The PEX gene was identified as being genetically linked to a disorder called X-linked hypophosphatemic rickets; a dominant disorder typified by decreased renal tubular phosphate reabsorption. Based on its close homology to the other members of the NEP family (49-60%) it is also predicted to be a membrane-bound metalloprotease, but no substrate has yet been found.

XCE (Valdenaire, O et al, Molecular Brain Research, Vol 64, 1999, pp. 211-221), and its rat equivalent DINE (Kiryu-Seo, S et al, Proceedings of the National Academy of Science USA, 2000, pp. 4345-4350), are expressed predominantly in the central nervous system. DINE expression is up-regulated following neuronal damage, and this is thought to help prevent neuronal apoptosis, possibly as a result of the DINE-mediated proteolytic activation of antioxidant enzymes. A physiological substrate of XCE/DINE has also not yet been identified, but from their sequence they are clearly predicted to be proteases, and for DINE, this has been proven using a synthetic peptide substrate.

Rodent SEP and NEPII were discovered most recently. NEPII is likely to be a rat equivalent of SEP, which is a mouse enzyme, as they share 91% amino acid identity. They are the members of this class closest to NEP in their amino acid sequence, both being 54% identical to human NEP. The mRNA of both is highly abundant in the testis and can also be detected at low levels in a broad range of other tissues. In the case of rat NEPII, the mRNA has also been found at comparatively high levels in the brain and pituitary. When produced recombinantly in mammalian cells, both mouse SEP and rat NEPII can be found in the growth media. This suggests they could be secreted proteases that may be able to circulate and hence cleave peptides at other sites in the body. Mouse SEP and rat NEPII, like some other members of this class such as ECE-1, exhibit splice variation. In the case of mouse SEP and rat NEPII, this splice variation results in isoforms with alterations in sequences involved in membrane localisation and secretion. The physiological significance of this is unclear but it is likely there could be membrane-bound, circulating, and intracellular forms of these enzymes. Mouse SEP has been shown to be able to cleave a range of important biological peptides including enkephalin, endothelin, big-endothelin, Bradykinin and substance P. Like NEP, therefore, it has a fairly broad substrate specificity and may have several physiological functions in different tissues.

Enzymes in this NEP class, like other metalloprotease enzymes, have been shown to be amenable to inhibition by small drug-like molecules (for example, thiorphan and phosphoramidon). This, together with the emerging nature of the physiological function of some members of the NEP-like enzymes in modulating peptidergic signalling, makes them attractive targets for pharmaceutical intervention. NEP inhibitors have been developed for indications including cardiovascular disease, and it is likely that, as knowledge of their function increases further, specific inhibitors of NEP-like enzymes may have a role in the prophylaxis and/or treatment of many other indications such as sexual dysfunction (especially male sexual dysfunction, e.g. male erectile dysfunction (MED), and female sexual dysfunction (FSD), e.g. female sexual arousal disorder (FSAD)), preterm labour, pre-eclampsia, endometriosis, reproductive disorders (especially male and female infertility, polycystic ovarian syndrome, implantation failure), hypertension, heart failure, angina, renal insufficiency, cyclical oedema, hyperaldosteroneism, glaucoma, asthma, inflammation, leukaemia, pain, epilepsy, affective disorders, dementia and geriatric confusion, obesity and gastrointestinal disorders (especially diarrhoea and irritable bowel syndrome), septic shock, the modulation of gastric acid secretion and the treatment of hyperreninaemia.

### SEXUAL DYSFUNCTION

Sexual dysfunction (SD) is a significant clinical problem, which can affect both males and females. The causes of SD may be both organic as well as psychological. Organic aspects of SD are typically caused by underlying vascular diseases, such as those associated with hypertension or diabetes mellitus, by prescription medication and/or by psychiatric disease such as depression. Physiological factors include fear, performance anxiety and interpersonal conflict. SD impairs sexual performance, diminishes self-esteem and disrupts personal relationships thereby inducing personal distress. In the clinic, SD disorders have been divided into female sexual dysfunction (FSD) disorders and male sexual dysfunction (MSD) disorders (Melman et al 1999). FSD is best defined as the difficulty or inability of a woman to find satisfaction in sexual expression. Male sexual dysfunction (MSD) is generally associated with erectile dysfunction, also known as male erectile dysfunction (MED) (Benet et al 1994).

The agents identified by the methods of the invention are particularly beneficial for the prophylaxis and/or treatment of sexual dysfunction in the male (e.g. male erectile dysfunction—MED) and in the female—female sexual dysfunction (FSD), e.g. female sexual arousal disorder (FSAD).

### FEMALE SEXUAL DYSFUNCTION (FSD)

FSD can be defined as the difficulty or inability of a woman to find satisfaction in sexual expression. FSD is a collective term for several diverse female sexual disorders (Leiblum, S. R. (1998). Definition and classification of female sexual disorders. Int. J. Impotence Res., 10, S104-S106; Berman, J. R., Berman, L. & Goldstein, I. (1999). Female sexual dysfunction: Incidence, pathophysiology, evaluations and treatment options. Urology, 54, 385-391.). The woman may have lack of desire, difficulty with arousal or orgasm, pain with intercourse or a combination of these problems. Several types of disease, medications, injuries or psychological problems can cause FSD. Treatments in development are targeted to treat specific subtypes of FSD, predominantly desire and arousal disorders.

The categories of FSD are best defined by contrasting them to the phases of normal female sexual response: desire, arousal and orgasm (Leiblum, S. R. (1998). Definition and classification of female sexual disorders. Int. J. Impotence Res., 10, S104-S106). Desire or libido is the drive for sexual expression. Its manifestations often include sexual thoughts either when in the company of an interested partner or when exposed to other erotic stimuli. Arousal is the vascular response to sexual stimulation, an important component of which is genital engorgement and includes increased vaginal lubrication, elongation of the vagina and increased genital sensation/sensitivity. Orgasm is the release of sexual tension that has culminated during arousal.

Hence, FSD occurs when a woman has an inadequate or unsatisfactory response in any of these phases, usually desire, arousal or orgasm. FSD categories include hypoactive sexual desire disorder, sexual arousal disorder, orgasmic disorders and sexual pain disorders. Improvement of the genital response to sexual stimulation (as in female sexual arousal disorder) may also improve the associated pain, distress and discomfort associated with intercourse and so treat other female sexual disorders.

Thus, in accordance with a preferred aspect of the invention, there is provided use of an agent identified by a method of the invention in the preparation of a medicament for the treatment or prophylaxis of hypoactive sexual desire disorder, sexual arousal disorder, orgasmic disorder or sexual pain disorders, more preferably for the treatment or prophylaxis of sexual arousal disorder, orgasmic disorder, or sexual pain disorders, and most preferably in the treatment or prophylaxis of sexual arousal disorder.

Hypoactive sexual desire disorder (HSDD) is present if a woman has no or little desire to be sexual, and has no or few sexual thoughts or fantasies. This type of FSD can be caused by low testosterone levels, due either to natural menopause or to surgical menopause. Other causes include illness, medications, fatigue, depression and anxiety.

Female sexual arousal disorder (FSAD) is characterised by inadequate genital response to sexual stimulation. The genitalia do not undergo the engorgement that characterises normal sexual arousal. The vaginal walls are poorly lubricated, so that intercourse is painful. Orgasms may be impeded. Arousal disorder can be caused by reduced oestrogen at menopause or after childbirth and during lactation, as well as by illnesses, with vascular components such as diabetes and atherosclerosis. Other causes result from treatment with diuretics, antihistamines, antidepressants e.g. selective serotonin re-uptake inhibitors (SSRIs) or antihypertensive agents.

Female orgasmic disorder (FOD) is characterised by persistent or recurrent delay in, or absence of, orgasm following a normal sexual excitement phase. Women exhibit wide variability in the type or intensity of stimulation that triggers orgasm. The diagnosis of FOD should be based on the clinician's judgement that the woman's orgasmic capacity is less than would be reasonable for her age, sexual experience, and the adequacy of the sexual stimulation she receives.

Sexual pain disorders (includes dyspareunia and vaginismus) is characterised by pain resulting from penetration and may be caused by medications which reduce lubrication, endometriosis, pelvic inflammatory disease, inflammatory bowel disease or urinary tract problems.

The prevalence of FSD is difficult to gauge because the term covers several types of problem, some of which are difficult to measure, and because the interest in treating FSD is relatively recent. Many women's sexual problems are associated either directly with the female ageing process or with chronic illnesses such as diabetes and hypertension.

Because FSD consists of several subtypes that express symptoms in separate phases of the sexual response cycle, there is not a single therapy. Current treatment of FSD focuses principally on psychological or relationship issues. Treatment of FSD is gradually evolving as more clinical and basic science studies are dedicated to the investigation of this medical problem. Female sexual complaints are not all psychological in pathophysiology, especially for those individuals who may have a component of vasculogenic dysfunction (e.g. FSAD) contributing to the overall female sexual complaint. There are at present no drugs licensed for the treatment of FSD. Empirical drug therapy includes oestrogen administration (topically or as hormone replacement therapy), androgens or mood-altering drugs such as buspirone or trazodone. These treatment options are often unsatisfactory due to low efficacy or unacceptable side effects.

Since interest is relatively recent in treating FSD pharmacologically, therapy consists of the following: psychological counselling, over-the-counter sexual lubricants, and investigational candidates, including drugs approved for other conditions. These medications consist of hormonal agents, either testosterone or combinations of oestrogen and testosterone and more recently vascular drugs that have proved effective in male erectile dysfunction (MED). None of these agents has yet been demonstrated to be effective in treating FSD.

As discussed, the agents identified by the methods of the invention are particularly useful for the prophylaxis and/or treatment of female sexual arousal disorder (FSAD).

The Diagnostic and Statistical Manual (DSM) IV of the American Psychiatric Association defines Female Sexual Arousal Disorder (FSAD) as being:

“. . . a persistent or recurrent inability to attain or to maintain until completion of the sexual activity adequate lubrication-swelling response of sexual excitement. The disturbance must cause marked distress or interpersonal difficulty . . . ”.

The arousal response consists of vasocongestion in the pelvis, vaginal lubrication and expansion and swelling of the external genitalia. The disturbance causes marked distress and/or interpersonal difficulty. Studies investigating sexual dysfunction in couples reveals that up to 76% of women have complaints of sexual dysfunction and that 30-50% of women in the USA experience FSD (Berman, J. R., Berman, L. A., Werbin, T. J. et al. (1999). Female sexual dysfunction: Anatomy, physiology, evaluation and treatment options. Curr Opin Urology, 9, 563-568).

FSAD is a highly prevalent sexual disorder affecting pre-, peri- and post-menopausal (± hormone replacement therapy (HRT)) women. It is associated with concomitant disorders such as depression, cardiovascular diseases, diabetes and urogenital (UG) disorders.

The primary consequences of FSAD are lack of engorgement/swelling, lack of lubrication and lack of pleasurable genital sensation. The secondary consequences of FSAD are reduced sexual desire, pain during intercourse and difficulty in achieving an orgasm.

It has recently been hypothesised that there is a vascular basis for at least a proportion of patients with symptoms of FSAD (Goldstein et al., Int. J. Impot. Res., 10, S84-S90, 1998) with animal data supporting this view (Park et al., Int. J. Impot. Res., 9, 27-37, 1997).

Without being bound by theory, we believe that neuropeptides such as vasoactive intestinal peptide (VIP) are major neurotransmitter candidates in the control of the female sexual arousal response, especially in the control of genital blood flow. VIP and other neuropeptides are degraded/metabolised by SEP. Thus, SEP inhibitors will potentiate the endogenous vasorelaxant effect of VIP released during arousal. This will lead to a prophylaxis and/or treatment of FSAD, such as through enhanced genital blood flow and thence genital engorgement. We have shown that inhibitors of SEP enhance pelvic nerve-stimulated and VIP-induced increases in vaginal and clitoral blood flow. In addition, we have shown that SEP inhibitors enhance VIP and nerve-mediated relaxations of isolated vagina wall.

Thus the present invention is advantageous as it helps provide a means for restoring a normal sexual arousal response—namely increased genital blood flow leading to vaginal, clitoral and labial engorgement. This will result in increased vaginal lubrication via plasma transudation, increased vaginal compliance and increased genital sensitivity. Hence, the present invention provides a means to restore, or potentiate, the normal sexual arousal response.

By female genitalia herein we mean: “The genital organs consist of an internal and external group. The internal organs are situated within the pelvis and consist of ovaries, the uterine tubes, uterus and the vagina. The external organs are superficial to the urogenital diaphragm and below the pelvic arch. They comprise the mons pubis, the labia majora and minora pudendi, the clitoris, the vestibule, the bulb of the vestibule, and the greater vestibular glands” (Gray's Anatomy, C. D. Clemente, 13^{th} American Edition).

R. J. Levin teaches us that because “. . . male and female genitalia develop embryologically from the common tissue anlagen, [that] male and female genital structures are argued to be homologues of one another. Thus the clitoris is the penile homologue and the labia homologues of the scrotal sac . . . ” (Levin, R. J. (1991), Exp. Clin. Endocrinol., 98, 61-69).

### MALE ERECTILE DYSFUNCTION (MED)

It is known that some individuals can suffer from male erectile dysfunction (MED).

Male erectile dysfunction (MED) is defined as:

“. . . the inability to achieve and/or maintain a penile erection for satisfactory sexual performance (NIH Consensus Development Panel on Impotence, 1993) . . .”.

It has been estimated that the prevalence of erectile dysfunction (ED) of all degrees (minimal, moderate and complete impotence) is 52% in men 40 to 70 years old, with higher rates in those older than 70 (Melman et al 1999). The condition has a significant negative impact on the quality of life of the patient and their partner; often resulting in increased anxiety and tension which leads to depression and low self-esteem. Whereas two decades ago, MED was primarily considered to be a psychological disorder (Benet et al 1994), it is now known that for the majority of patients there is an underlying organic cause. As a result, much progress has been made in identifying the mechanism of normal penile erection and the pathophysiology of MED.

Penile erection is a haemodynamic event, which is dependent upon the balance of contraction and relaxation of the corpus cavernosal smooth muscle and vasculature of the penis (Lemer et al 1993). Corpus cavernosal smooth muscle is also referred to herein as corporal smooth muscle or in the plural sense corpus cavernosa. Relaxation of the corpus cavemosal smooth muscle leads to an increased blood flow into the trabecular spaces of the corpus cavernosa, causing them to expand against the surrounding tunica and compress the draining veins. This produces a vast elevation in blood pressure which results in an erection (Naylor, 1998).

The changes that occur during the erectile process are complex and require a high degree of co-ordinated control involving the peripheral and central nervous systems, and the endocrine system (Naylor, 1998). Corporal smooth muscle contraction is modulated by sympathetic noradrenergic innervation via activation of postsynaptic α₁ adrenoceptors. MED may be associated with an increase in the endogenous smooth muscle tone of the corpus cavernosum. However, the process of corporal smooth muscle relaxation is mediated partly by non-adrenergic, non-cholinergic (NANC) neurotransmission. There are a number of other NANC neurotransmitters found in the penis, other than nitric oxide (NO), such as calcitonin gene related peptide (CGRP) and vasoactive intestinal peptide (VIP). The main relaxing factor responsible for mediating this relaxation is NO, which is synthesised from L-arginine by nitric oxide synthase (NOS) (Taub et al 1993; Chuang et al 1998). It is thought that reducing corporal smooth muscle tone may aid NO to induce relaxation of the corpus cavernosum. During sexual arousal in the male, NO is released from neurones and the endothelium and binds to and activates soluble guanylate cyclase (sGC) located in the smooth muscle cells and endothelium, leading to an elevation in intracellular cyclic guanosine 3′,5′-monophosphate (cGMP) levels. This rise in cGMP leads to a relaxation of the corpus cavernosum due to a reduction in the intracellular calcium concentration ([Ca²⁺]ᵢ), via unknown mechanisms thought to involve protein kinase G activation (possibly due to activation of Ca²⁺ pumps and Ca²⁺-activated K⁺ channels; Chuang et al., 1998).

Sildenafil citrate (also known as Viagram™) has recently been developed by Pfizer as the first oral drug treatment for MED. Sildenafil acts by inhibiting cGMP breakdown in the corpus cavemosa by selectively inhibiting phosphodiesterase 5 (PDE5), thereby limiting the hydrolysis of cGMP to 5′GMP (Boolel et al., 1996; Jeremy et al., 1997) and thereby increasing the intracellular concentrations of cGMP and facilitating corpus cavernosal smooth muscle relaxation.

Currently, all other available MED therapies on the market, such as treatment with prostaglandin based compounds i.e. alprostadil which can be administered intra-urethrally (available from Vivus Inc., as Muse™) or via small needle injection (available from Pharmacia & Upjohn, as Caverject™), are either inconvenient and/or invasive. Other treatments include vacuum constriction devices, vasoactive drug injection or penile prostheses implantation (Montague et al., 1996). Although injectable vasoactive drugs show high efficacy, side effects such as penile pain, fibrosis and priapism are common, and injection therapy is not as convenient as oral therapy therefore sildenafil currently represents the most preferred therapy on the market.

There is no prior documented evidence for the expression or a functional role of SEP in the penis or corpus cavernosum or in the erectile mechanism/process.

There is also no prior documented evidence for a functional or biochemical effect for SEP inhibitors on the penis or corpus cavernosum or alternatively in the erectile mechanism/process.

There is no prior documented evidence for the expression or a functional role of SEP in the vagina or clitoral corpus cavernosum or in the female sexual arousal mechanism/process.

There is also no prior documented evidence for a functional or biochemical effect for SEP inhibitors in the vagina or clitoral corpus cavemosum or in the female sexual arousal mechanism/process.

Thus, a seminal finding described herein is the ability to treat a male or a female suffering from sexual dysfunction, in particular MED or FSAD, with use of a soluble secreted endopeptidase inhibitor (SEPi). Surprisingly the applicants have also found that inhibition of SEP with a SEPi significantly enhances the nerve-stimulated arousal process.

The present invention is advantageous as it provides methods for identifying agents that are able to restore or potentiate a normal sexual arousal response—namely increased penile blood flow leading to erection of the penis in males and a increased vaginal clitoral blood flow leading to genital engorgement in females.

References

Argiolas, A. et al (1995), Neuromodulation of penile erection. Prog Neurobiol. 47: 235-255.

Benet, A. E. et al (1994), Male erectile dysfunction assessment and treatment options. Comp. Ther. 20: 669-673.

Boolel, M. et al (1996). Sildenafil, a novel effective oral therapy for male erectile dysfunction. Br. J. of Urology 78: 257-261.

Carter A J. et al (1998). Effect of the selective phosphodiesterase type 5 inhibitor sildenafil on erectile dysfunction in the anesthetized dog. J. Urol. 160: 242-6.

Chiou, W. F. et al (1998). Relaxation of corpus cavemosum and raised intracavemous pressure by berberine in rabbit. Br. J. Pharmacol. 125: 1677-1684.

Jeremy, J. Y. et al (1997). Effects of sildenafil, a type-5 cGMP phosphodiesterase inhibitor, and papaverine on cyclic GMP and cyclic AMP levels in the rabbit corpus cavernosum in vitro. Br. J. Urology 79: 958-963.

Lemer, S. E. et al (1993). A review of erectile dysfunction: new insights and more questions. J. Urology 149: 1246-1255.

Melman,A. & Gingell, J. C. (1999). The epidemiology and pathophysiology of erectile dysfunction. J. Urology 161: 5-11.

Montague, D. et al (1996). Clinical guidelines panel on erectile dysfunction: Summary report on the treatment of organic erectile dysfunction. J. Urology 156: 2007-2011.

Naylor, A. M. (1998). Endogenous neurotransmitters mediating penile erection. Br. J. Urology 81: 424-431.

NIH Consensus Development Panel on Impotence (1993). NIH Consensus Conference Impotence. J.A.M.A. 270: 83.

Omote M. (1999). Pharmacological profiles of sildenafil (VIAGRA) in the treatment of erectile dysfunction: efficacy and drug interaction with nitrate. Nippon Yakurigaku Zasshi. 114:213-8.

Taub, H. C. et al (1993). Relationship between contraction and relaxation in human and rabbit corpus cavernosum. Urology 42: 698-704.

Traish A M, et al (1999). Effects of castration and androgen replacement on erectile function in a rabbit model. Endocrinology. 140: 1861-8.

### SUMMARY OF THE INVENTION

In a broad aspect, the present invention relates to modulators (especially inhibitiors) of novel amino acid sequences and methods to indentify them. Whilst the modulators and methods of the invention relate to a specific novel amino acid sequence that has been identified (see WO 02/06492—filed 16 Jul. 2001), it is to be understood that the invention refers to that sequence as well as novel variants, fragments, derivatives and homologues thereof. The modulators and methods of the invention also relate to other amino acids of the same or similar type/family.

Thus, in brief, some aspects of the present invention relate to:

Assay methods using novel sequences.

Novel assay methods using peptidases generally, specific peptidases and, more specifically, novel amino acids.

Agents/compounds identified by use of said assay methods.

As used herein, “amino acid sequence” refers to peptide or protein sequences and may refer to portions thereof. In addition, the term “amino acid sequence” is synonymous with the phrase “polypeptide sequence”. Also, the term “nucleotide sequence” is synonymous with the phrase “polynucleotide sequence”.

Other features and advantages of the invention will be apparent from the following detailed description and from the claims. While the invention is described in connection with specific embodiments, it will be understood that other changes and modifications that may be practiced are also part of this invention and are also within the scope of the appendant claims. This application is intended to cover any equivalents, variations, uses, or adaptations of the invention that follow, in general, the principles of the invention, including departures from the present disclosure that come within known or customary practice within the art, and that are able to be ascertained without undue experimentation. Additional guidance with respect to making and using nucleic acids and polypeptides is found in standard textbooks of molecular biology, protein science, and immunology (see, e.g., Davis et al., Basic Methods in Molecular Biology, Elsevir Sciences Publishing, Inc., New York, N.Y., 1986; Hames et al., Nucleic Acid Hybridization, IL Press, 1985; Molecular Cloning, Sambrook et al., Current Protocols in Molecular Biology, Eds. Ausubel et al., John Wiley and Sons; Current Protocols in Human Genetics, Eds. Dracopoli et al., John Wiley and Sons; Current Protocols in Protein Science, Eds. John E. Coligan et al., John Wiley and Sons; and Current Protocols in Immunology, Eds. John E. Coligan et al., John Wiley and Sons). All publications mentioned herein are incorporated by reference in their entireties.

### DESCRIPTION OF THE FIGURES

FIG. 1 shows an analysis of open reading frames (ORFs) of human SEP cDNA sequence.

FIG. 2 shows a comparison of human SEP to most closely related human proteins by pairwise alignment from the blastp algorithm.

FIG. 3 shows a comparison of human, rat and mouse sequences for SEP by pairwise alignment from blastp (protein) and fasta (coding nucleotide) algorithms.

FIG. 4 shows multiple alignment of human SEP and related human proteins showing catalytic domain.

FIG. 5 shows multiple alignment of human, rat and mouse SEP proteins showing catalytic domain.

FIG. 6 shows multiple alignment of human, rat and mouse SEP coding sequence showing catalytic domain.

FIG. 7 shows a phylogenetic analysis by Neighbour-Joining Distance method expressed as a radial tree derived from multiple alignment of whole SEP-like proteins.

FIG. 8 shows a phylogenetic analysis by Neighbour-Joining Distance method expressed as a radial tree derived from the catalytic domain region of the multiple alignment.

FIG. 9 shows the FRET-based assay principle. Increased fluorescence is detected in the presence of the substrate peptide cleaving enzyme (“+ Enzyme”) compared to the fluorescence level detected in the absence of the substrate peptide cleaving enzyme (“− Enzyme”). In the presence of an appropriate enzyme inhibitor, the fluorescence level detected would be similar to that detected in the absence of the substrate peptide cleaving enzyme (“− Enzyme”). Intensity=fluorescence intensity. Wavelength=in nm (nanometres).

FIG. 10 shows a plot of Assay Value Ratio (AVR) results over time on Robolab.

FIG. 11 shows the second validation experiment. The percentage cut-off for selection of primary actives was set at 30%. “+ Spike”=addition of known inhibitor compound. “−Spike”=no inhibitor compound added.

FIG. 12 shows a plot of Assay Value Ratio (AVR) results during primary screening. “⋄” represents one AVR plate.

FIG. 13 shows distribution of results in the primary screen. The hit rate for actives was 2.2%.

FIG. 14 shows some screening statistics.

FIG. 15 shows an example of an IC₅₀ curve from a confirmed active. The data in general was of a high standard, reflecting the quality of the assay. RFU=relative fluorescence units. [I}uM=[I] μM=concentration of inhibitor (I) in micromoles.

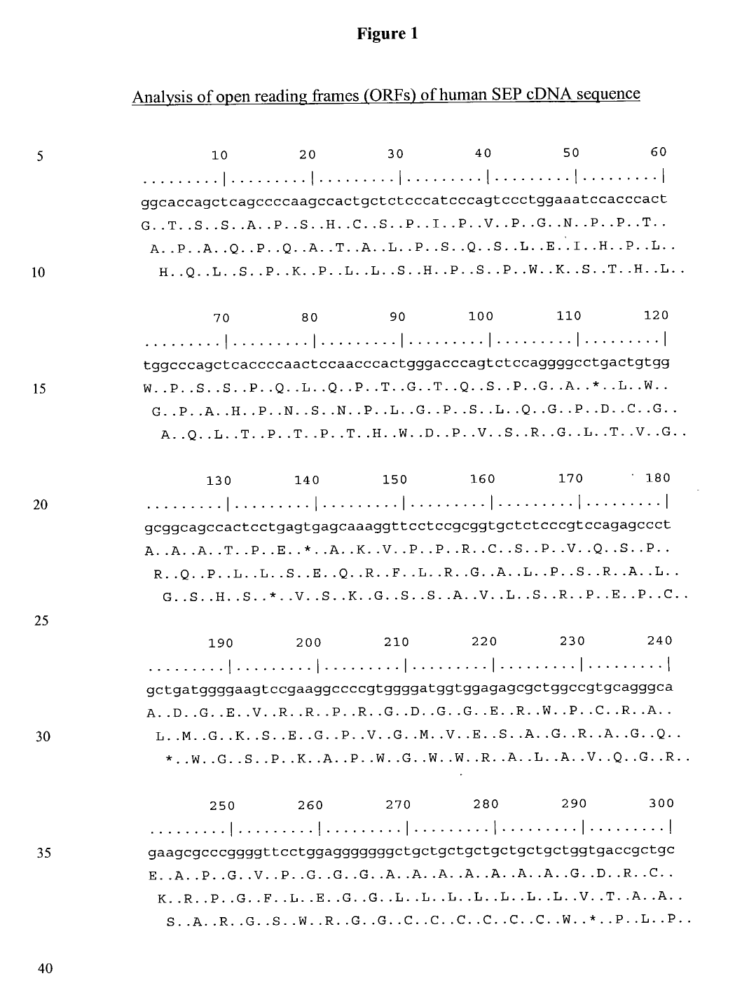

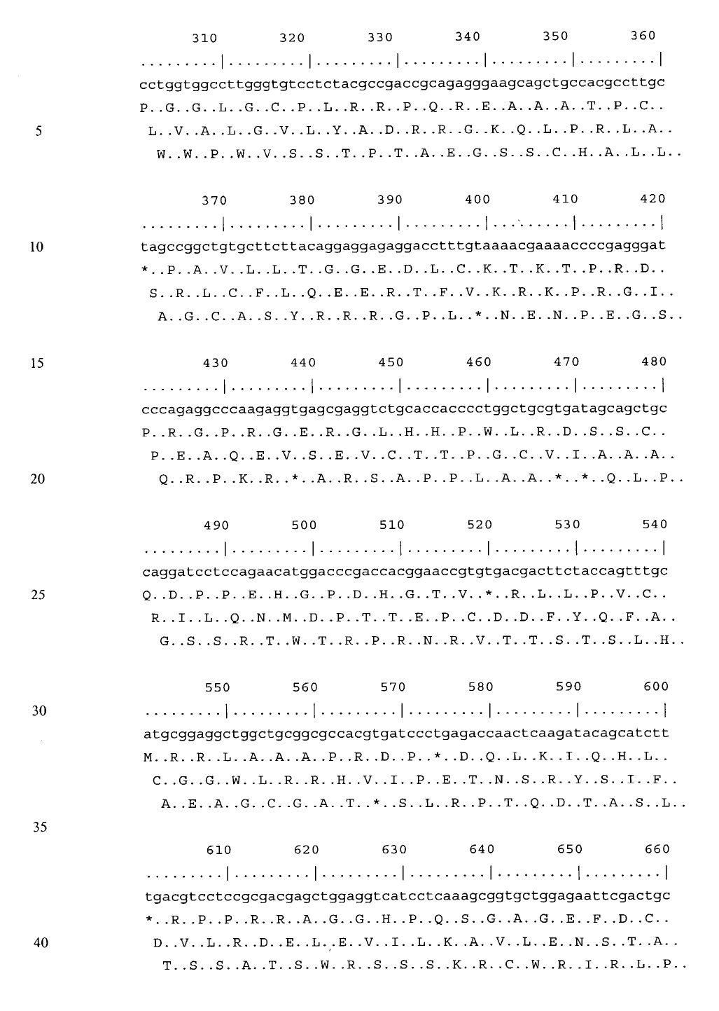

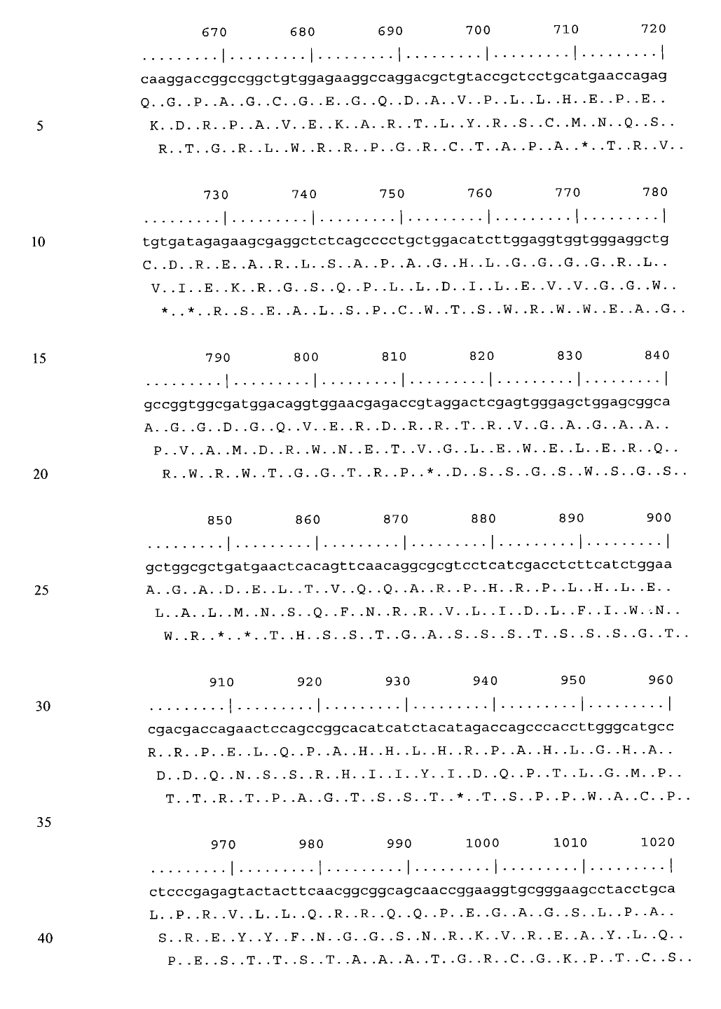

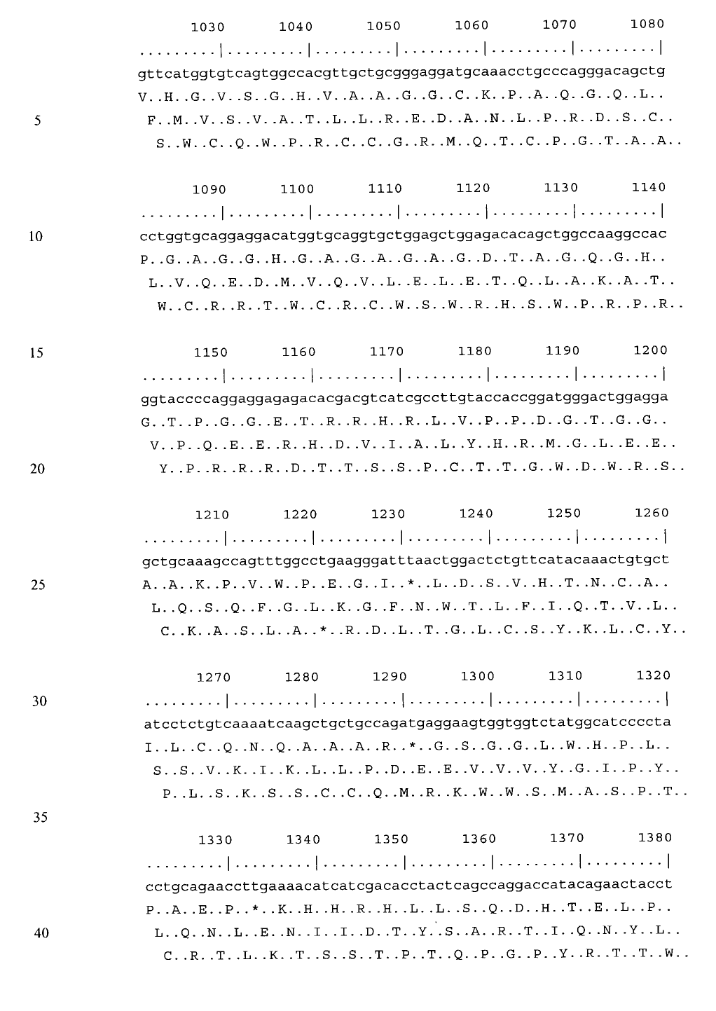

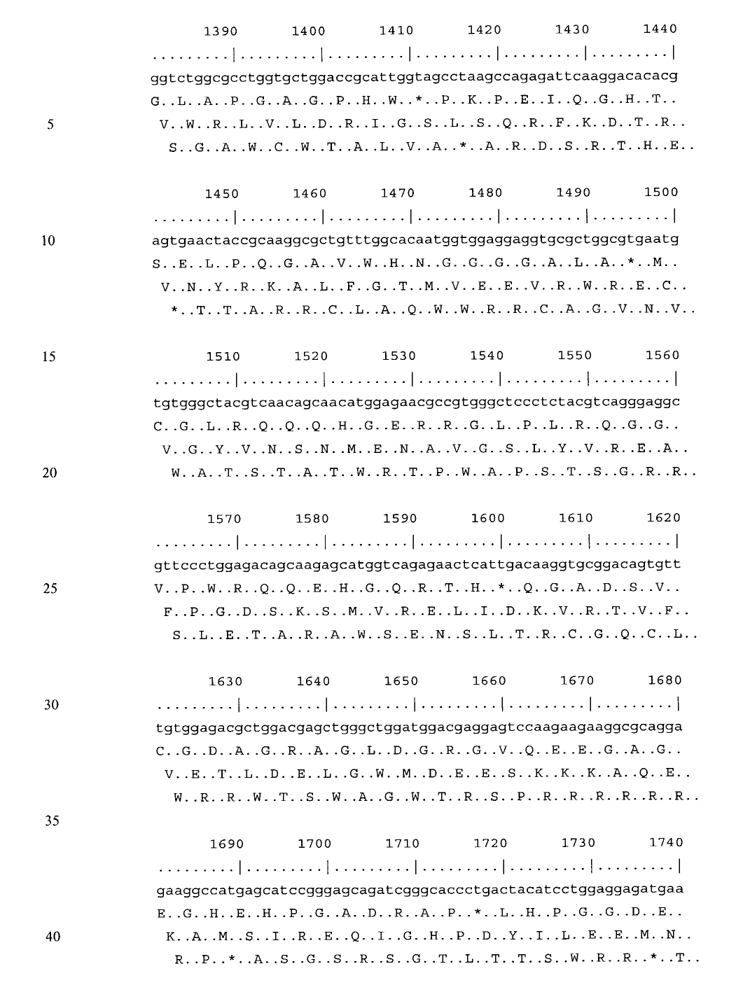

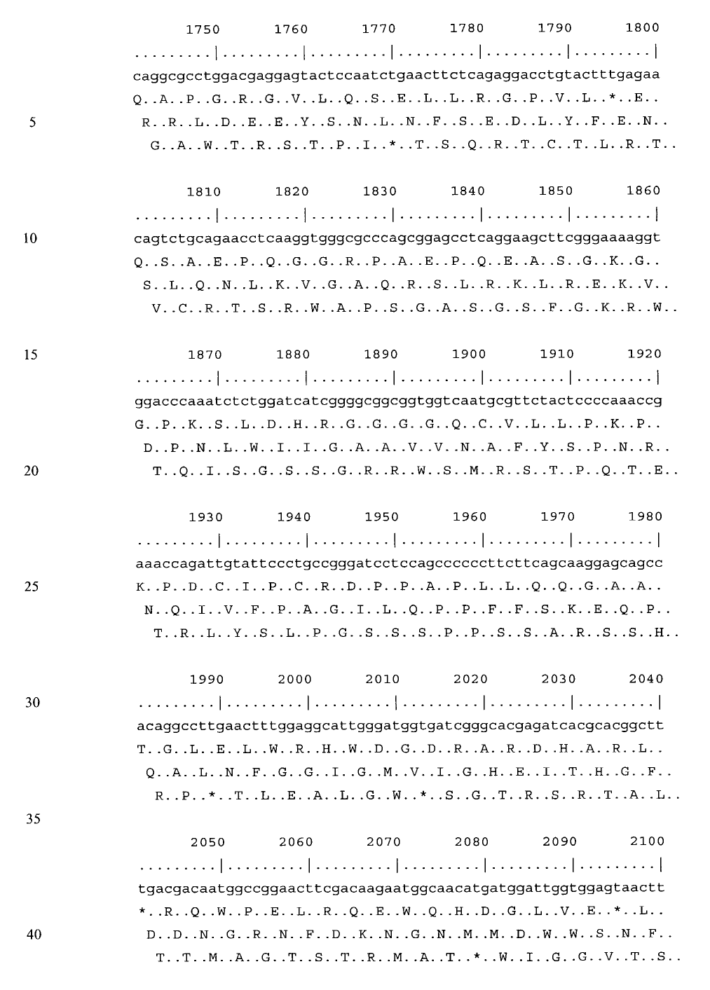

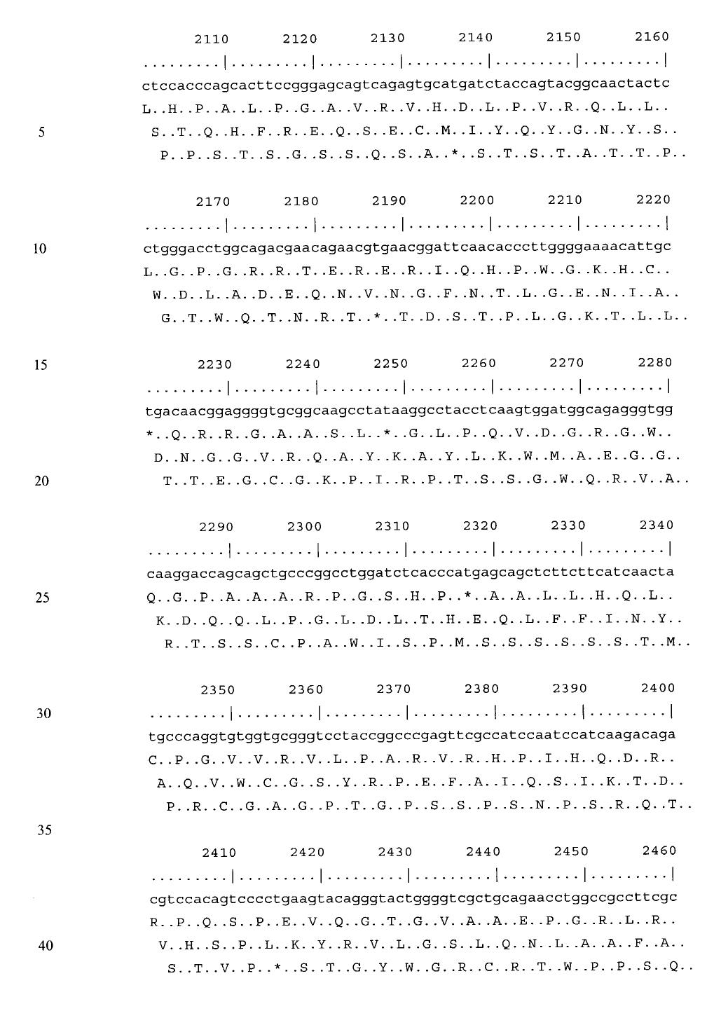

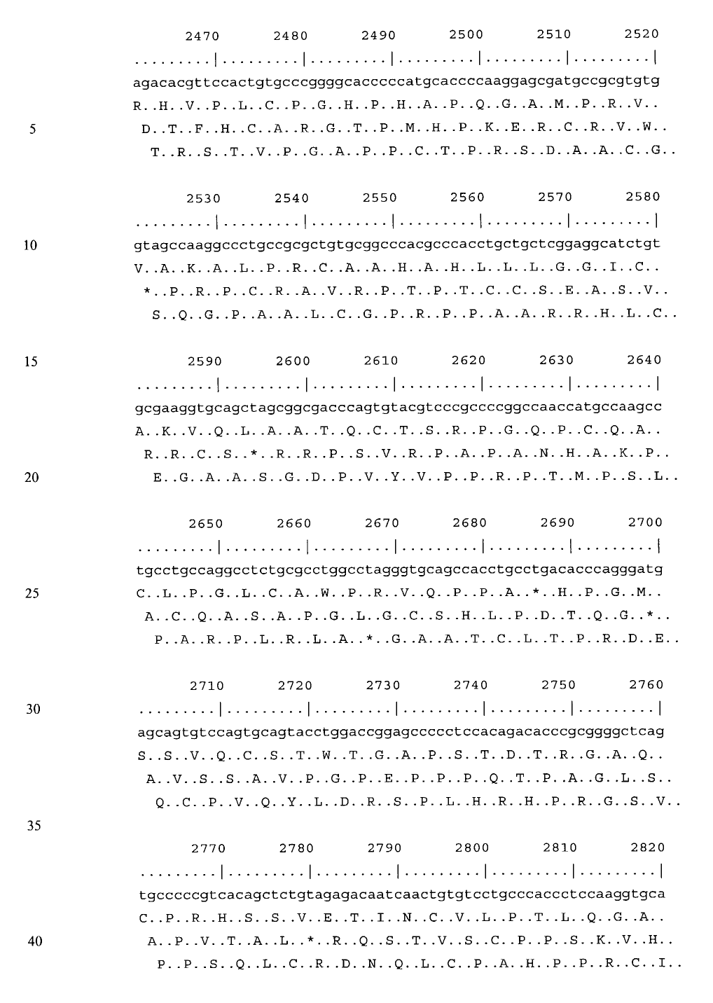

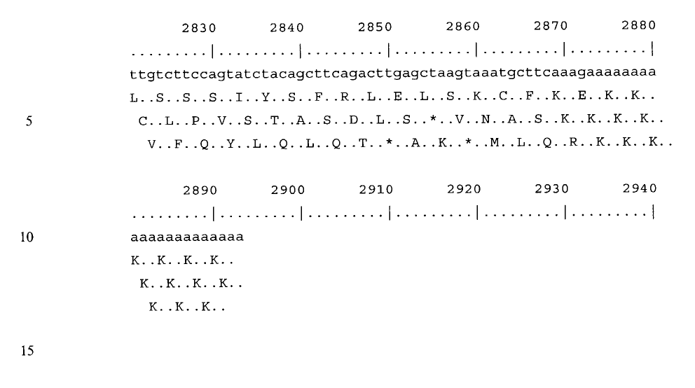

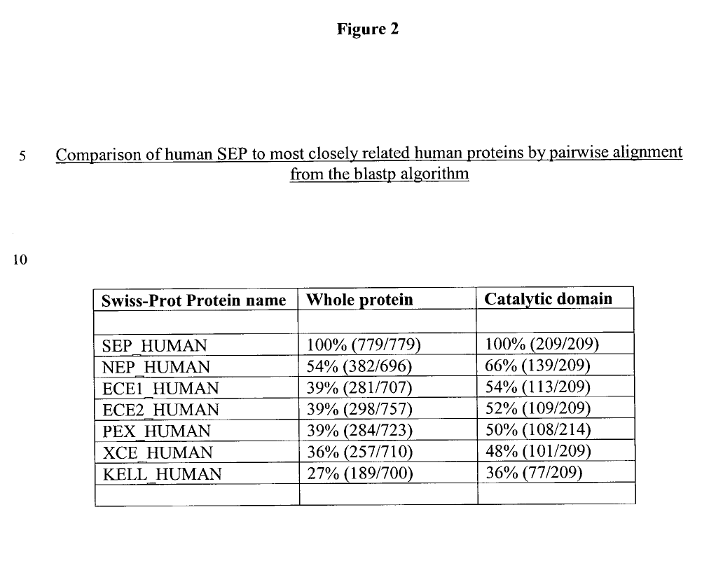

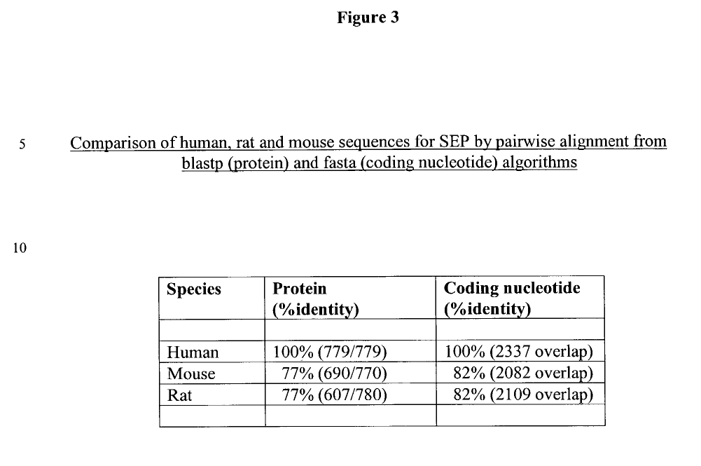

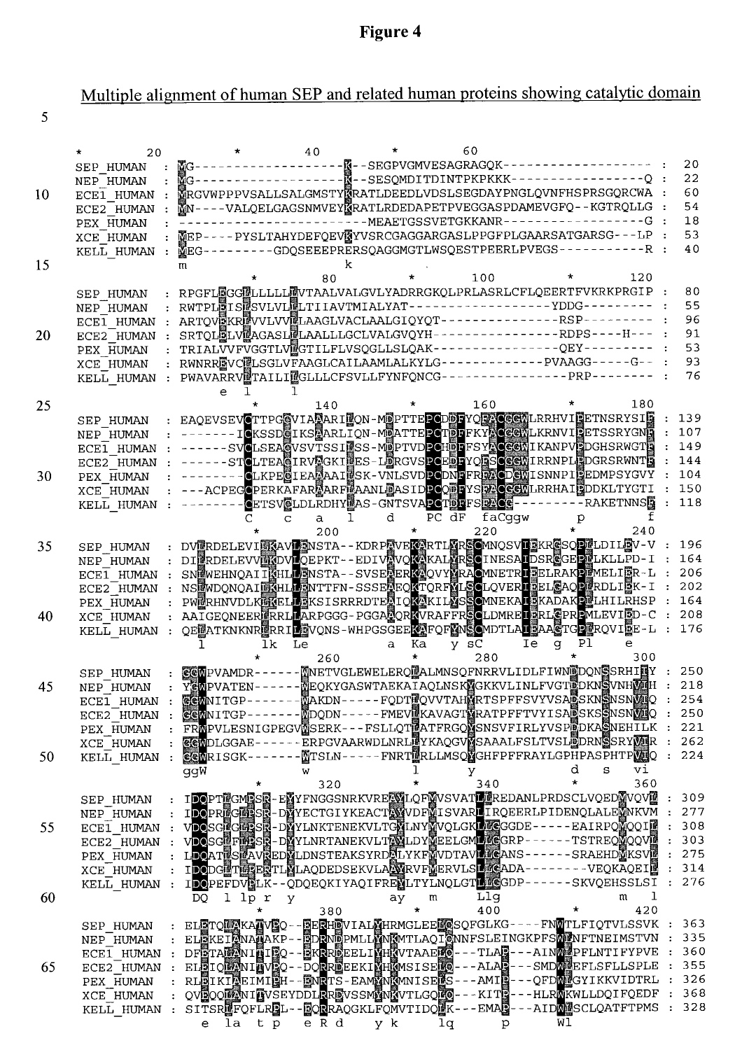

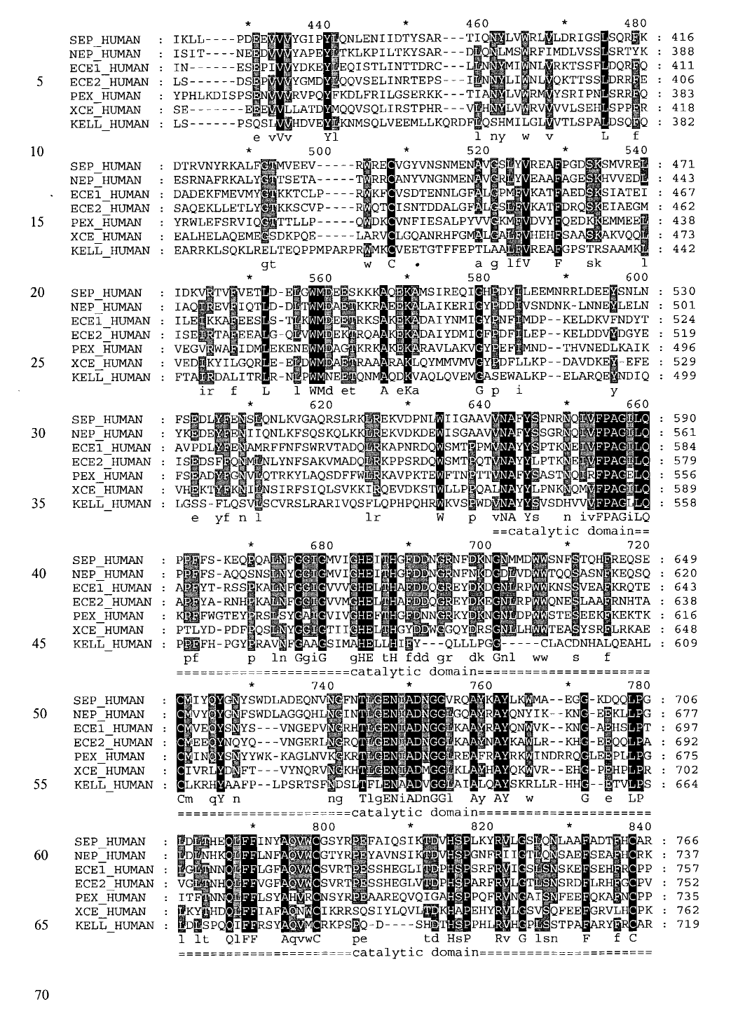

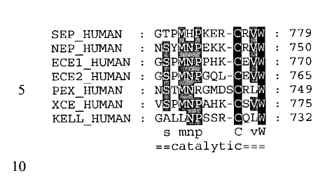

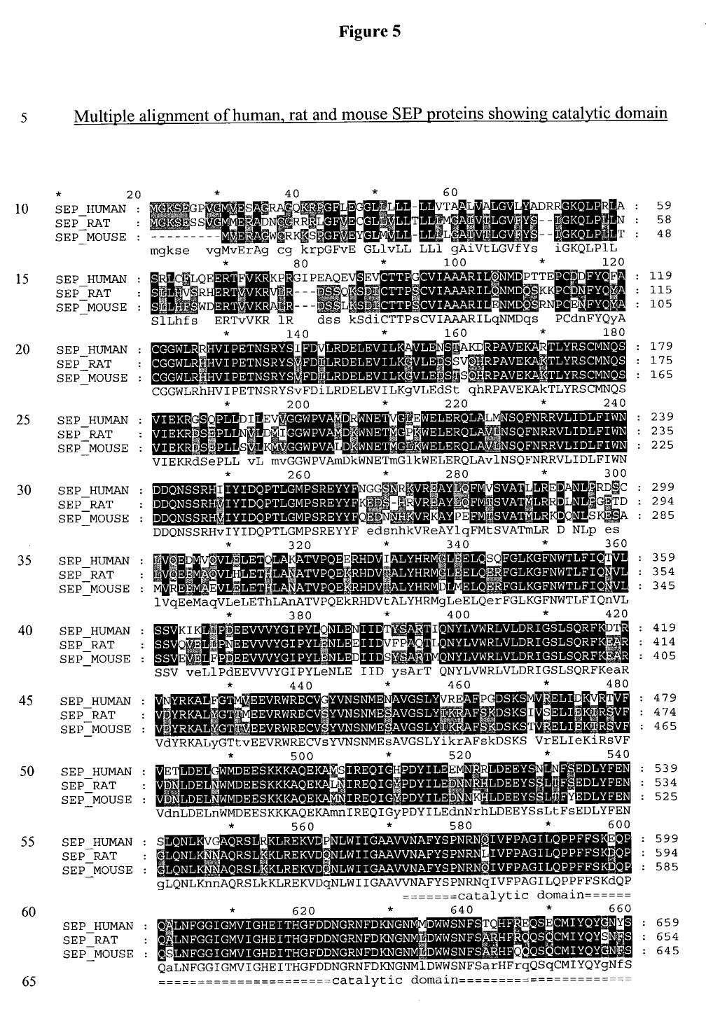

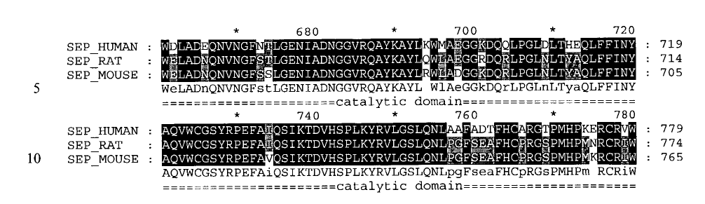

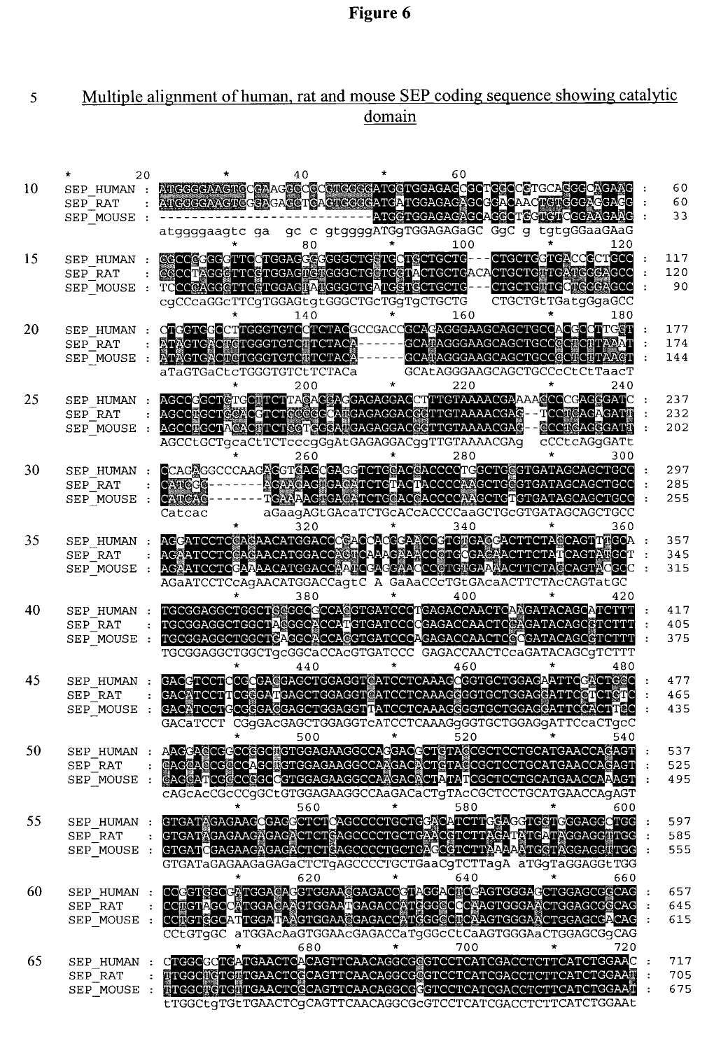

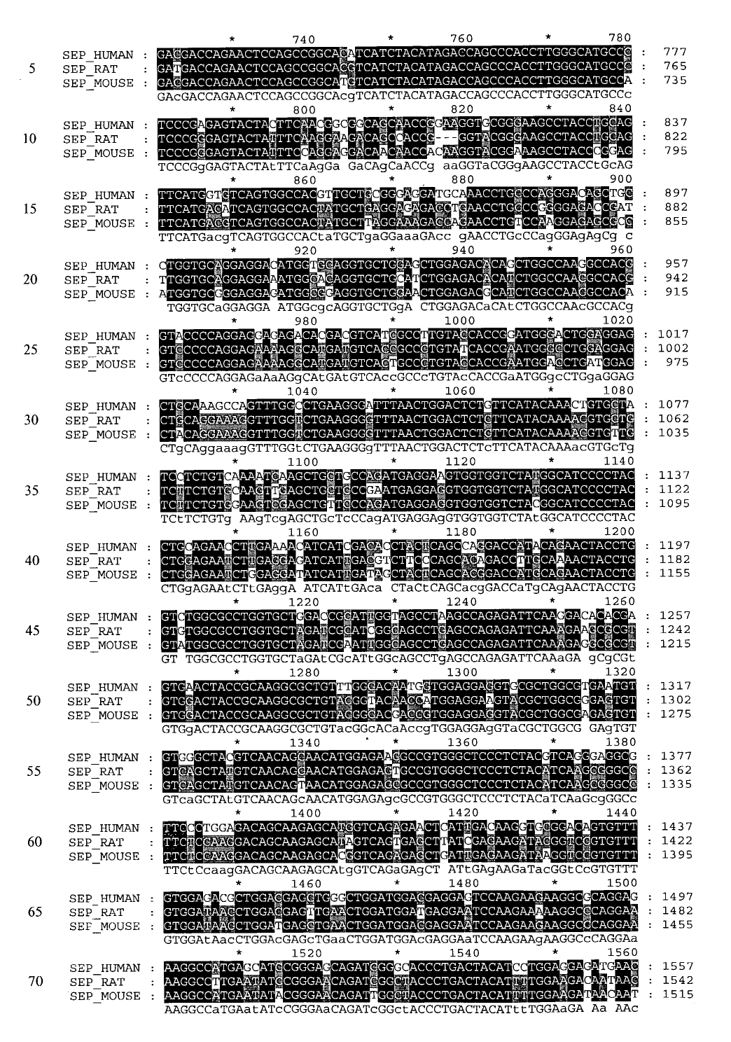

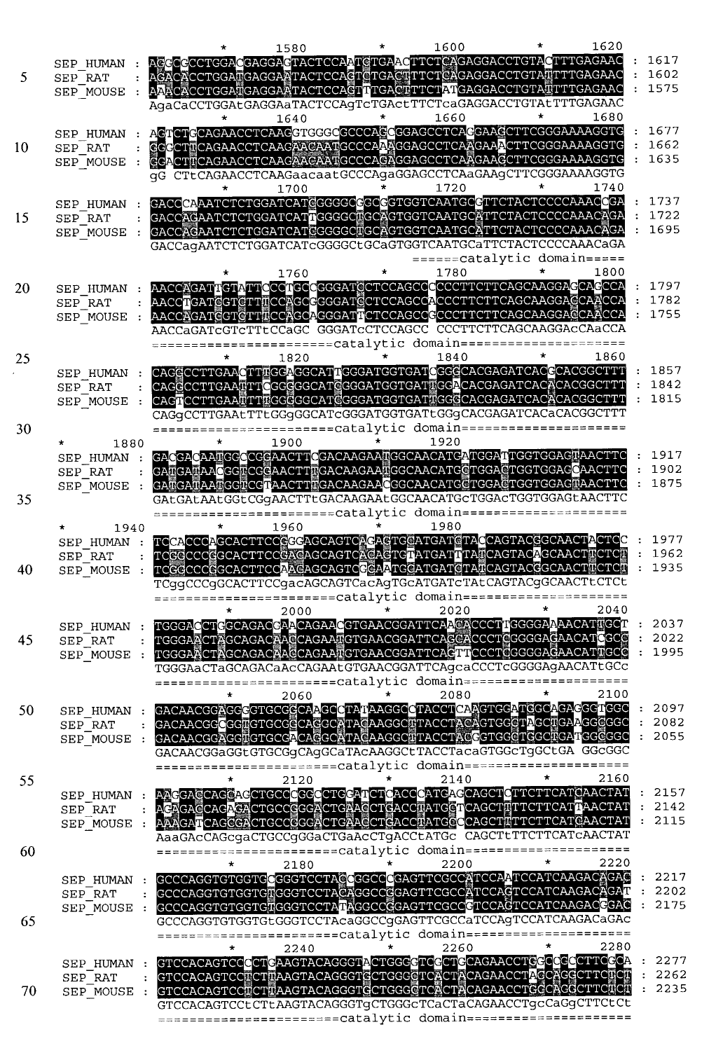

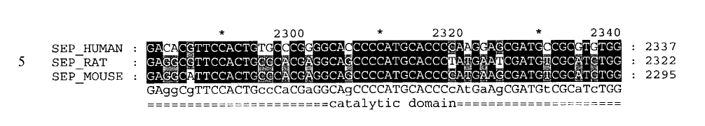

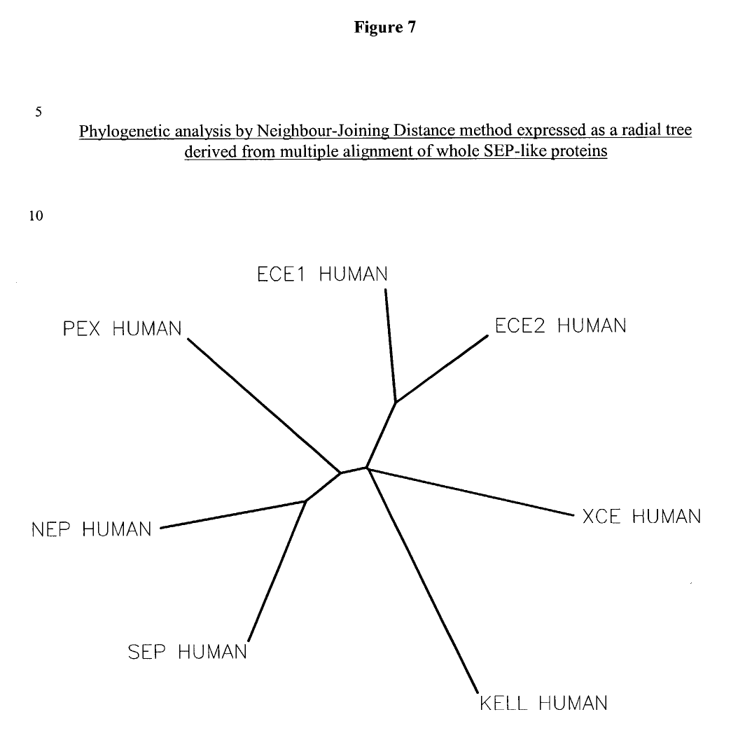

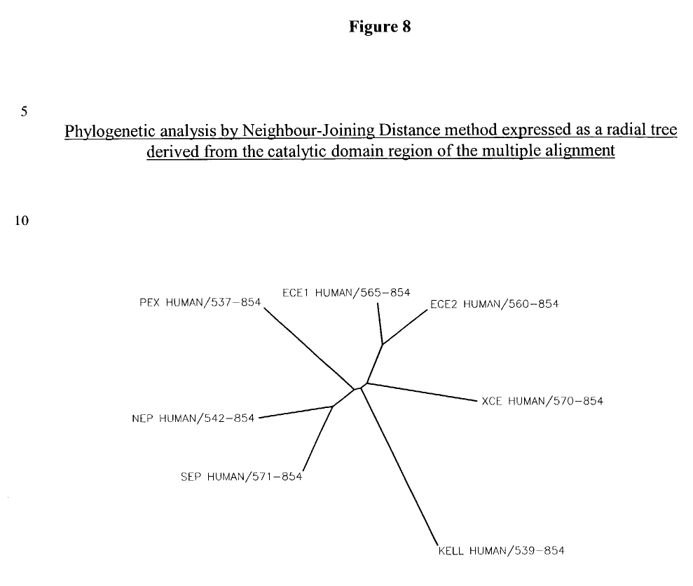

### DETAILED DESCRIPTION

According to a first aspect of the present invention, there is provided a method for identifying an agent which binds to and/or modulates a SEP polypeptide comprising contacting said agent with a polypeptide comprising: SEQ ID NO: 2, an amino acid sequence encoded by the polynucleotide sequence of SEQ ID NO: 1 or SEQ ID NO: 5; or a polypeptide encoded by the cDNA of NCIMB 41110, and determining whether binding and/or modulation occurs.

Preferably, said SEP polypeptide comprises one or more of:

(a) a polypeptide having the deduced amino acid sequence translated from the polynucleotide sequence in SEQ ID NO: 1 or SEQ ID NO: 5 and variants, fragments, homologues, analogues and derivatives thereof;

(b) a polypeptide of SEQ ID NO: 2 and variants, fragments, homologues, analogues and derivatives thereof;

(c) a polypeptide encoded by the cDNA of NCIMB 41110 and variants, fragments, homologues, analogues and derivatives thereof; or

(d) a polypeptide which has at least 78% identity to (i) the polypeptide encoded by the polynucleotide of SEQ ID NO: 1 or SEQ ID NO: 5, (ii) the polypeptide of SEQ ID NO: 2, or (iii) the polypeptide encoded by the cDNA of NCIMB 41110.

Preferably, the polypeptide comprises a polypeptide sequence that has at least 85%, preferably at least 90%, more preferably at least 95%, most preferably at least 98% identity to (i) the polypeptide encoded by the polynucleotide of SEQ ID NO: 1 or SEQ ID NO: 5, (ii) the polypeptide of SEQ ID NO: 2, or (iii) the polypeptide encoded by the cDNA of NCIMB 41110.

Preferably, the polypeptide described above comprises the amino acid sequence MGKSEGPVG (SEQ ID NO: 6). Preferably said amino acid sequence MGKSEGPVG (SEQ ID NO: 6) is at or near the amino terminus of the polypeptide.

Preferably, the polynucleotide which encodes the polypeptide described above comprises the nucleotide sequence ATGGGGAAGTCCGAAGGCCCCGTGGGG (SEQ ID NO: 7). Preferably said nucleotide sequence ATGGGGAAGTCCGAAGGCCCCGTGGGG (SEQ ID NO: 7) is at or near the 5′ end of the polynucleotide.

Said SEP polypeptide is described in more detail in WO 02/06492 (filed 16 Jul. 2001), which is incorporated herein by reference in its entirety.

Preferably, the method according to the first aspect of the invention comprises contacting said SEP polypeptide with a SEP substrate peptide in the presence of said agent, wherein said substrate peptide is capable of providing a detectable signal in response to cleavage by said SEP polypeptide, wherein said agent is identified as a modulator of said SEP polypeptide if there is a difference in the detectable signal in the presence and in the absence of said agent. More preferably, said method identifies an agent that decreases said detectable signal and is a SEP polypeptide inhibitor. Even more preferably, said substrate peptide is labelled with at least one fluorescent donor dye and said signal is detected by Fluorescence Resonance Energy Transfer (FRET) assay. Yet more preferably, said labelled substrate peptide is Rhodamine green-Gly-Gly-dPhe-Leu-Arg-Arg-Val-CyS(QSY™7)-βAla-NH₂, 5-(and 6) tetramethyl rhodamine Gly-Gly-dPhe-Leu-Arg-Arg-Val-CyS(QSY™7)-βAla-NH₂ or 5-carboxyfluorescein-Gly-Gly-dPhe-Leu-Arg-Arg-Val-Cys(5-(and 6)tetramethylrhodamine)-βAla-NH₂. Preferably, binding between said SEP polypeptide and said agent is detected. Most preferably, said method is a competitive binding assay.

According to a second aspect of the present invention, there is provided a method for identifying an agent which inhibits or selectively inhibits a peptidase comprising contacting said peptidase and a peptidase substrate peptide selected from the group consisting of Rhodamine green-Gly-Gly-dPhe-Leu-Arg-Arg-Val-Cys(QSY™7)-βAla-NH₂, 5-(and 6) tetramethyl rhodamine Gly-Gly-dPhe-Leu-Arg-Arg-Val-Cys(QSY™7)-βAla-NH₂ and 5-carboxyfluorescein-Gly-Gly-dPhe-Leu-Arg-Arg-Val-Cys (5- (and 6) tetramethylrhodamine)-βAla-NH₂, in the presence of said agent, wherein said substrate peptide is capable of providing a detectable signal in response to cleavage by said peptidase as detected by Fluorescence Resonance Energy Transfer (FRET) assay, wherein said agent is identified as an inhibitor of said peptidase if there is a decrease in the detectable signal in the presence of said agent as compared to in the absence of said agent.

Preferably, said peptidase is an exopeptidase or an endopeptidase. More preferably, said peptidase is oxytocinase, neutral endopeptidase (NEP), or non-human SEP.

According to a third aspect of the present invention, there is provided an agent identified by any of the methods described above; wherein said agent is not phosphoramidon, thiorphan, fasidotrilat, omapatrilat or FR901533. When SEP modulators are sought, preferably said agent modulates the SEP polypeptide described above. More preferably, the compound inhibits or selectively inhibits the SEP polypeptide.

Preferably, said agent identified by any of the methods used to identify SEP modulators has a greater than 30-fold selectivity (preferably greater than 50-fold selectivity) for SEP over neutral endopeptidase NEP EC 3.4.24.11 and angiotensin converting enzyme (ACE).

Preferably, said agent identified by any of the methods used to identify SEP modulators has a greater than 100-fold selectivity for SEP over endothelin converting enzyme (ECE).

Also provided by the present invention is a pharmaceutical composition comprising the agent identified by the methods described above and one or more pharmaceutically acceptable carriers, diluents or excipients.

According to another aspect of the invention, there is provided a method for identifying a candidate modulator, which binds to and/or modulates the SEP polypeptide described above comprising contacting said SEP polypeptide with a candidate modulator and determining whether modulation occurs.

Preferably, said method comprises:

(a) contacting (i) a substrate peptide of the SEP polypeptide described above with (ii) the SEP polypeptide, in the presence of (iii) a candidate modulator of the SEP polypeptide, wherein said substrate peptide is capable of providing a detectable signal in response to cleavage of said substrate peptide by the SEP polypeptide; and

(b) determining whether the cleavage of said substrate peptide by the SEP polypeptide has been modulated or not by said candidate modulator by detecting the presence or absence of said detectable signal associated with said substrate peptide.

Preferably, said candidate modulator is a candidate inhibitor. More preferably, said substrate peptide is labelled with at least one fluorescent donor dye and at least one fluorescence acceptor dye and the assay used to detect candidate inhibitors of the polypeptide described above is a Fluorescence Resonance Energy Transfer (FRET) assay. Most preferably, said labelled substrate peptide is Rhodamine green-Gly-Gly-dPhe-Leu-Arg-Arg-Val-CyS(QSY™-7)-βAla-NH₂ (CP4) (SEQ ID NO: 8) or 5-(and 6) tetramethyl rhodamine Gly-Gly-dPhe-Leu-Arg-Arg-Val-Cys(QSY™-7)-βAla-NH₂ (CP5) (SEQ ID NO: 8) or 5-carboxyfluorescein-Gly-Gly-dPhe-Leu-Arg-Arg-Val-Cys (5- (and 6) tetramethylrhodamine)-βAla-NH₂ (CP6) (SEQ ID NO: 8).

Also contemplated by the present invention are FRET assays using the labelled substrate peptide CP4, CP5, or CP6, wherein the SEP polypeptide described above is replaced by any peptidase. Preferably, said peptidase is an exopeptidase or an endopeptidase. More preferably, said exopeptidase is oxytocinase and said endopeptidase is NEP or non-human SEP.

Thus, according to a preferred aspect of the present invention, there is provided a method for identifying a candidate inhibitor which binds to and/or inhibits the SEP polypeptide described above, which comprises:

(a) contacting (i) a substrate peptide of the SEP polypeptide described above with (ii) the SEP polypeptide, in the presence of (iii) a candidate inhibitor of the SEP polypeptide, wherein said substrate peptide is capable of providing a detectable signal in response to cleavage of said substrate peptide by the SEP polypeptide; and

(b) determining whether the cleavage of said substrate peptide by the SEP polypeptide has been inhibited or not by said candidate inhibitor by detecting the presence or absence of said detectable signal associated with said substrate peptide.

Preferably, said substrate peptide is labelled with at least one fluorescent donor dye and at least one fluorescence acceptor dye and the assay used to detect candidate inhibitors of the polypeptide described above is a Fluorescence Resonance Energy Transfer (FRET) assay. Most preferably, said labelled substrate peptide is CP4, CP5, or CP6.

Also contemplated by the present invention are FRET assays using the labelled substrate peptide CP4, CP5, or CP6, wherein the SEP polypeptide described above is replaced by any peptidase. Preferably, said peptidase is an exopeptidase or an endopeptidase. More preferably, said exopeptidase is oxytocinase and said endopeptidase is NEP or non-human SEP.

The FRET assays of the present invention are based on an assay developed by Carvalho et al. for use with NEP (Carvalho et al., Annal. Biochem. 237, pp. 167-173 (1996)). The FRET assays of the invention utilise a similar intramolecularly quenched fluorogenic peptide substrate, but with a novel combination of fluorogenic donor/acceptor dyes.

The preparation of the synthetic labelled substrate peptides, e.g., CP4, CP5, and CP6, are further detailed below.

According to an alternative aspect of the present invention, there is provided a method for identifying a candidate modulator, which binds to and/or modulates the SEP polypeptide described above comprising contacting said SEP polypeptide with a candidate modulator and determining whether modulation occurs; wherein said binding between the SEP polypeptide and said candidate modulator is detected.

Preferably, said method comprises:

(a) contacting a candidate modulator with cells secreting the SEP polypeptide described above, said SEP polypeptide being associated with a second component capable of providing a detectable signal in response to the binding of a candidate modulator to said SEP polypeptide; said contacting being under conditions sufficient to permit binding of candidate modulators to the SEP polypeptide; and

(b) identifying a candidate modulator capable of SEP polypeptide binding by detecting the signal produced by said second component.

Preferably, said method is a competitive binding assay. More preferably, said method comprises:

(a) contacting (i) a detectable first component known to bind to the SEP polypeptide described above and (ii) a candidate modulator, with cells secreting the above SEP polypeptide, said SEP polypeptide being associated with a second component capable of providing a detectable signal in response to the binding of a candidate modulator to said SEP polypeptide; said contacting being under conditions sufficient to permit binding of candidate modulators to the SEP polypeptide; and

(b) determining whether the first component binds to the SEP polypeptide by detecting the absence or otherwise of a signal generated from the interaction of the first component with the SEP polypeptide.

Also contemplated by the present invention are FRET assays using the labelled substrate peptide CP4, CP5, or CP6, wherein the SEP polypeptide described above is replaced by any peptidase. Preferably, said peptidase is an exopeptidase or an endopeptidase. More preferably, said exopeptidase is oxytocinase and said endopeptidase is NEP or non-human SEP.

According to another aspect of the present invention, there is provided a candidate modulator identified by any of the methods described above. Preferably, said modulator binds to and/or inhibits or selectively inhibits the SEP polypeptide or peptidases described above.

In a further aspect, the present invention relates to an assay method for identifying an agent (hereinafter referred to as a SEP inhibitor or SEPi) that can be used to treat female sexual dysfunction, in particular FSAD, or male sexual dysfunction, in particular MED, the assay method comprising: determining whether a test agent can directly enhance the endogenous genital engorgement process or erectile process; wherein said enhancement is defined as a potentiation of genital blood flow or intracavemosal pressure (ICP) (and/or cavemosal blood flow) in the presence of a test agent as defined herein; such potentiation by a test agent is indicative that the test agent may be useful in the prophylaxis and/or treatment of female sexual dysfunction, in particular FSAD, or male sexual dysfunction, in particular MED and wherein said test agent is a SEPi.

By way of example, the present invention relates to an assay method for identifying an agent that can directly enhance the endogenous genital arousal or erectile process in order to treat female sexual dysfunction, in particular FSAD, or male sexual dysfunction, in particular MED, the assay method comprising: contacting a test agent which has a moiety capable of inhibiting the metabolic breakdown of a peptide (preferably a fluorescent labelled peptide, such as Rhodamine green-Gly-Gly-dPhe-Leu-Arg-Arg-Val-CyS(QSY™7)-βAla-NH₂) (SEQ ID NO: 8) or the other fluorescent labelled peptides described above, said peptide being normally metabolised by SEP; and measuring the activity and/or levels of peptide remaining after a fixed time (for example via fluorometric analysis); wherein the change in the level of the fluorescence by the cleavage product peptide Rhodamine green-Gly-Gly-dPhe-Leu-Arg-Arg-OH is indicative of the potency (IC₅₀) of the test agent and is indicative that the test agent may be useful in the prophylaxis and/or treatment of female sexual dysfunction, in particular FSAD, or male sexual dysfunction, in particular MED; and wherein said agent is a SEPi.

In a further aspect, the present invention relates to a process comprising the steps of: (a) performing the assay according to the present invention; (b) identifying one or more agents that can directly enhance the endogenous genital arousal process or erectile process; and (c) preparing a quantity of those one or more identified agents; and wherein said agent is a SEPi.

With this aspect, the agent identified in step (b) may be modified so as to, for example, maximise activity and then step (a) may be repeated. These steps may be repeated until the desired activity or pharmacokinetic profile has been achieved.

Thus, in a further aspect, the present invention relates to a process comprising the steps of: (a1) performing the assay according to the present invention; (b1) identifying one or more agents that can directly enhance the endogenous genital arousal process or erectile process; (b2) modifying one or more of said identified agents; (a2) optionally repeating step (a1); and (c) preparing a quantity of those one or more identified agents (i.e. those that have been modified); and wherein said agent is a SEPi.

Thus, in accordance with a preferred aspect of the invention, there is provided use of an agent identified by any of the methods of the invention described herein in the manufacture of a medicament for the treatment or prophylaxis of female sexual dysfunction (FSD), in particular FSAD, or male sexual dysfunction, in particular MED. Treatment or prophylaxis of other FSDs are also contemplated, including: hypoactive sexual desire disorder (HSDD), female orgasmic disorder (FOD) and sexual pain disorders (in particular dyspareunia or vaginismus).

It is to be understood that any candidate modulator, candidate inhibitor, candidate selective inhibitor, agent (hereinafter referred to as a SEP inhibitor or SEPi), etc. that is identified using any assay (or modification thereof) described above is also deemed an aspect of the present invention. Moreover, it is also to be understood that any candidate modulator, candidate inhibitor, candidate selective inhibitor, agent, etc., which is capable of modulating (preferably inhibiting or selectively inhibiting) any peptidase and that is identified using any assay (or modification thereof) described above is also deemed an aspect of the present invention. Preferably, said candidate modulator, candidate inhibitor, candidate selective inhibitor, agent, etc. modulates (preferably inhibits or selectively inhibits) an exopeptidase or an endopeptidase. More preferably, said exopeptidase is oxytocinase and said endopeptidase is NEP or non-human SEP.

As endopeptidases are involved in, inter alia, regulating bioactive peptide activity (e.g. peptidergic signalling processes), modulators (e.g. inhibitors, including selective inhibitors) of endopeptidases can find use in modulating such activity.

Some endopeptidases, such as ECE-1, are involved in proteolysis of biologically inactive peptides into their active form. Therefore, modulators (e.g. inhibitors, including selective inhibitors) of endopeptidases can find use in modulating such activity.

Human SEP, inter alia, may therefore be involved in regulating bioactive peptide activity and/or in proteolysis of biologically inactive peptides into their active form.

Consequently, the present invention provides an agent identified by any of the methods described above for use as a pharmaceutical. Such agents, which can act as modulators (preferably inhibitors or selective inhibitors) of the SEP polypeptide or peptidases described above, can therefore find use in the therapeutic areas that concern aspects of regulating bioactive peptide activity, such as modulating peptidergic signalling processes and/or in proteolysis of biologically inactive peptides into their active form. Such therapeutically usefully areas include, but are not limited to, sexual dysfunction (e.g. female sexual dysfunction, in particular FSAD, or male sexual dysfunction, in particular MED), fertility disorders, neurodegenerative disorders such as stroke, cardiovascular diseases such as hypertension, wound healing/tissue repair, etc.

The term “inhibitor” (and the like) in the context of the present invention means an agent that can substantially prevent the molecule which is being “inhibited” from carrying out its usual biological function/action. Such an “inhibitor” may inhibit more than one type of molecule, i.e. it can be a “non-selective inhibitor”.

The term “selective inhibitor” (and the like) in the context of the present invention means an agent that can substantially prevent the molecule which is being “inhibited” from carrying out its usual biological function/action. Such an “inhibitor” generally inhibits only one type of molecule or a limited number of types of molecules. Some “selective inhibitors” are, of course, more selective than others.

For example, when the agent of the invention is a SEP inhibitor (SEPi), said SEPi's of the present invention have an IC₅₀ at less than 100 nanomolar, more preferably, at less than 50 nanomolar.

Preferably, the SEP inhibitors according to the present invention have greater than 30-fold, more preferably greater than 50-fold selectivity for SEP over neutral endopeptidase NEP EC 3.4.24.11 and angiotensin converting enzyme (ACE). This reduces the prospect of cardiovascular events (e.g. drop in blood pressure) when the SEPi is administered systemically (e.g. by mouth). Preferably the SEPi also has a greater than 100-fold selectivity over endothelin converting enzyme (ECE).

SEPi compounds are prepared according to the teachings presented in the Experimental section (infra). They are tested as agents and are found to be useful for enhancing the endogenous erectile process, and thereby being useful in the prophylaxis and/or treatment of MED and FSAD.

Without being limited to any particular theory it is proposed herein that by inhibiting SEP, other neuronally released vasoactive agents (most likely vasoactive intestinal protein (VIP)), that are released during sexual arousal, are enhanced. It is believed that use of the SEPi potentiates the effects of neuropeptides (most likely VIP) that are released during sexual stimulation, and hence potentiates the male erectile mechanism by increasing cavernosal blood flow and thus intracavemosal pressure and female engorgement by increasing genital blood flow.

It is further proposed that the use of the compounds according to the present invention acts via enhancing a non-NO-dependant NANC pathway to treat MED and FSAD, and to potentiate or facilitate the nitrergic signalling in the penis and vagina/clitoris.

In our studies we have developed a robust reproducible model of the physiology of male and female sexual arousal. This model uses an anaesthetised rabbit and employs Laser Doppler technologies to monitor intracavernosal pressure and genital blood flow whilst routinely recording cardiovascular parameters. We are capable of measuring small changes in intracavernosal pressure within the penis and vaginal (and even clitoral) blood flow induced by pelvic nerve stimulation or infusion of VIP in the absence and presence of test agents.

We believe that our animal model directly reflects the clinical data. Hence, this model can be used to study candidate agents for the prophylaxis and/or treatment of e.g. MED and FSAD, such as measuring enhancement of penile erection via increases in intracavernosal pressure and enhancement of vaginal or clitoral blood flow.

In a further aspect, the present invention relates to an assay method for identifying an agent that can directly enhance the endogenous genital arousal process or erectile process in order to treat FSAD or MED, the assay method comprising: administering an agent to the animal model of the present invention; and measuring the change in the endogenous genital arousal process or erectile process; wherein said change is defined as a potentiation of vaginal/clitoral blood flow, intracavemosal pressure (ICP) (and/or cavernosal blood flow) in the animal model in the presence of a test agent as defined; and wherein said agent is a SEPi.

In a further aspect, the present invention relates to a diagnostic method, the method comprising isolating a sample from a female or male; determining whether the sample contains an entity present in such an amount as to cause female sexual dysfunction, preferably FSAD, or male sexual dysfunction, preferably MED; wherein the entity has a direct effect on the endogenous genital arousal process in the female or erectile process in the corpus cavemosum of the male; and wherein said entity can be modulated to achieve a beneficial effect by use of an agent; and wherein said agent is a SEPi.

In a further aspect, the present invention relates to a diagnostic composition or kit comprising means for detecting an entity in an isolated female or male sample; wherein the means can be used to determine whether the sample contains the entity and in such an amount to cause female sexual dysfunction, preferably FSAD, or male sexual dysfunction, preferably MED, or is in an amount so as to cause sexual dysfunction, preferably FSAD or MED; wherein the entity has a direct effect on the endogenous genital arousal process or erectile process and wherein said entity can be modulated to achieve a beneficial effect by use of an agent; and wherein said agent is a SEPi.

For ease of reference, these and further aspects of the present invention are now discussed under appropriate section headings. However, the teachings under each section are not necessarily limited to each particular section.

### HUMAN SEP ENZYME

As explained above, the present invention relates to the use of a novel endopeptidase enzyme—which has been designated human soluble secreted endopeptidase (human SEP)—and to a nucleotide sequence encoding same in assay methods. The present invention also relates to the use of the novel nucleic acid and amino acid sequences in methods to identify agents useful in the diagnosis and prophylaxis and/or treatment of disease. The present invention specifically relates to the use of the novel nucleic acid and amino acid sequences to evaluate and/or to screen for agents that can modulate (preferably inhibit or selectively inhibit) endopeptidase activity, preferably human SEP activity.

Human SEP is believed to be present in, and obtainable from, a variety of sources. By way of example, human SEP is found in any one or more of the cardiovascular system, the neurological system, the endocrine system and the testis.

We also believe that SEP is also present in a number of other sources—such as, for example: rodent (murine (Ikeda et al., Journal Biological Chemistry, Vol 274, 1999, pp.32469-32477) and rat (NEP II—International Patent Application WO 99/53077)), bovine, ovine, porcine, and equine.

The human SEP may be the same as the naturally occurring form—for this aspect, preferably the human SEP is the non-native amino acid sequence (i.e. it is not present in its natural environment)—or is a variant, homologue, fragment or derivative thereof In addition, or in the alternative, the human SEP is isolated human SEP and/or purified human SEP. The human SEP can be obtainable from, or produced by, any suitable source, whether natural or not, or it may be synthetic, semi-synthetic or recombinant.

The human SEP coding sequence may be the same as the naturally occurring form—for this aspect, preferably the human SEP coding sequence is the non-native nucleotide sequence (i.e. it is not present in its natural environment)—or is a variant, homologue, fragment or derivative thereof. In addition, or in the alternative, the human SEP coding sequence is an isolated human SEP coding sequence and/or a purified human SEP coding sequence. The human SEP coding sequence can be obtainable from, or produced by, any suitable source, whether natural or not, or it may be synthetic, semi-synthetic or recombinant.

### HUMAN SEP ACTIVITY AND SCREENING

Human SEP and/or its coding sequence and/or a sequence capable of hybridising thereto is/are useful for testing the selectivity of drug candidates between different SEPs.

Human SEP is believed to be able to hydrolyse (proteolyse) various bioactive peptides.

It has been demonstrated (herein) that human SEP mRNA is most abundant in the testis compared to other tissues. This is in line with data published for mouse SEP (Ghaddar et al., Biochemical Journal, Vol 347, 2000, pp. 419-429), where the mRNA has been further localized to round and elongated spermatids.

Multiple different proteolytic activities have been identified in the testis previously, and in some cases they have been shown to be essential for testis function (e.g. ACE activity). It is likely from its abundance in this tissue that one possible physiological role of SEP will be related to a function of the testis (e.g. fertility).

Human SEP may hydrolyse a particular physiologically important biological peptide within the testis that is involved in an aspect of fertility or another function of the testis to either activate, or inactivate the peptide. The exact nature of this (predicted) physiological peptide has yet to be determined. However SEP can hydrolyse many biological peptides including big-endothelin, endothelin-1, angiotensin-I, substance P, bradykinin, enkephalins, and atrial natriuretic peptide (ANP). Several of these are known to function in the testis. Endothelin-1, for example, is found in the testis where it is involved in promoting seminiferous tubule contractility. Interestingly, the enzyme activity (ECE) detected within the testis that is thought to generate the endothelin-1 from big-endothelin is sensitive to the inhibitors phosphoramidon and thiorphan. Since mouse SEP and human SEP are also sensitive to these inhibitors, this activity may be related to SEP rather than ECE. Compounds that inhibit human SEP activity may therefore lead to altered (increased or decreased) levels of endothelin within the testis, and this could potentially be useful for the prophylaxis and/or treatment of infertility or as a male contraceptive.

Vasoactive intestinal peptide (VIP) is another potential human SEP substrate that plays a role in the testis. VIP has been shown to increase blood flow in the testis and also to promote testicular steroidogenesis, as well as human sperm motility (Siow et al, Archives of Andrology, Vol 43, 1999, p. 6771). It is possible that drugs inhibiting human SEP activity could be useful in modulating VIP levels in the testis and hence testicular bloodflow, steroidogenesis or sperm motility.

Although human SEP mRNA is most abundant in testis, it can also be detected at lower levels in a variety of tissues (e.g. salivary gland and thyroid gland) using the sensitive method of reverse transcriptase-polymerase chain reaction (RT-PCR). In the mouse, SEP has been detected in heart, brain, spleen, lungs, kidney, intestine, and adrenal gland. The cDNA library screening approach that was used herein to isolate the full length human SEP cDNA sequence also identified human SEP cDNAs in libraries derived from human brain, placenta, small intestine and kidney tissue.

Human SEP enzyme can also be secreted from the cell. It is likely therefore that the human SEP protein, in addition to being abundant in the testis, will also be found at moderate levels in a wide range of other tissues throughout the body. In these tissues it is likely that human SEP will hydrolyse the biological peptide substrates mentioned above.

Drugs that inhibit the enzyme activity of SEP will therefore be likely to lead to changes in the levels of many of the human SEP substrates mentioned above in a variety of different tissues. As these human SEP substrates are usually biologically active molecules or their precursors which are often associated with peptidergic signalling processes, it is likely that human SEP inhibitors may be useful for the prophylaxis and/or treatment of many different disorders associated with peptidergic signalling that will most likely include, but is not limited to, sexual dysfunction (e.g. female sexual dysfunction, in particular FSAD, or male sexual dysfunction, in particular MED) and reproductive disorders, as well as other diseases/disorders such as neurodegenerative disorders (e.g. stroke), and cardiovascular diseases such as hypertension, etc.

Thus, human SEP and/or its coding sequence and/or a sequence capable of hybridising thereto may be useful for screening drug candidates for the prophylaxis and/or treatment of diseases associated with peptidergic signalling. In addition, it is believed that human SEP and/or its coding sequence and/or a sequence capable of hybridising thereto may be useful for screening drug candidates for the prophylaxis and/or treatment of diseases such as those described above.

Either or both of the nucleotide sequences coding for human SEP or the human SEP enzyme itself may be used to screen for agents that can affect SEP activity. In particular, the nucleotide sequence coding for human SEP itself may be used to screen for agents that can inhibit SEP activity. In addition, the nucleotide sequence coding for human SEP or the human SEP enzyme itself may be used to screen for agents that selectively affect SEP activity, such as selectively inhibit SEP activity.

### MEASUREMENT OF HUMAN SEP ACTIVITY—HUMAN SEP ASSAYS

The enzymatic (proteolytic) activity of human SEP protein can be measured in an assay involving, for example, mixing a sample of the human SEP enzyme with a substrate peptide in a buffer solution (for example 50 mM HEPES; pH 7.4), incubating the mixture for a period of time (such as 1-3 hours) enough for human SEP to act to cleave a measurable portion of the peptide substrate to a product at a temperature suitable for SEP activity (typically 30-37° C.). Thereafter, the substrate and/or products of the proteolysis can be analysed to demonstrate that the substrate has been cleaved by the SEP enzyme.

The effect of candidate human SEP inhibitor compounds or control test compounds such as phosphoramidon and thiorphan that may alter the activity of human SEP can be measured in this type of assay by including them in the initial mixture at a range of suitable test concentrations, typically 0.1 nM to 50 μM.

Samples of SEP enzyme suitable for use in the above type of assay can be produced using a recombinant expression system. This will typically involve introducing an expression plasmid containing the human SEP cDNA or gene (e.g. the expression vector obtainable from NCIMB 41110) into a host organism or cell where the human SEP protein is then expressed. SEP protein may be released (i.e. secreted extracellularly) from the host into the growth media (e.g. if artificially expressed in mammalian cells), or retained in the cell (e.g. if artificially expressed in yeast or insect cells—where possible improper expression could result in the failure of human SEP to be secreted from the cell, therefore necessitating isolation from its intracellular location). Typically the host can be a yeast, insect cell, mammalian cell, or bacteria.

The SEP enzyme can then be recovered from the culture media or host cell (e.g. by lysing the cells) which may necessitate using protein purification methods.

Human SEP enzyme for the aforementioned assay may also be purified from a suitable tissue source (if a sufficient quantity is obtainable). This tissue can include testis or brain.

Substrates suitable for use in the human SEP assay can be any peptide that SEP is able to cleave at a rate which is measurable in a useful period of time, e.g. 5 hours. Such substrate peptides can include, but are not limited to, peptides which are the same as, or similar to, biological peptides such as enkephalin, VIP, bradykinin, substance P, big endothelin, endothelin, angiotensin-I or ANP. The peptide can be modified to include a fluorescent, coloured, radioactive, or other chemical group that will facilitate measurement of the substrate and/or products before, during, or after the assay.

Preferred substrates suitable for use in the human SEP assay are SEP-cleavable synthetic peptides labelled with at least one fluorescent donor dye and at least one fluorescence acceptor dye and the assay used to detect inhibition of said SEP cleavage (proteolysis) is a Fluorescence Resonance Energy Transfer (FRET) assay.

### FRET ASSAYS

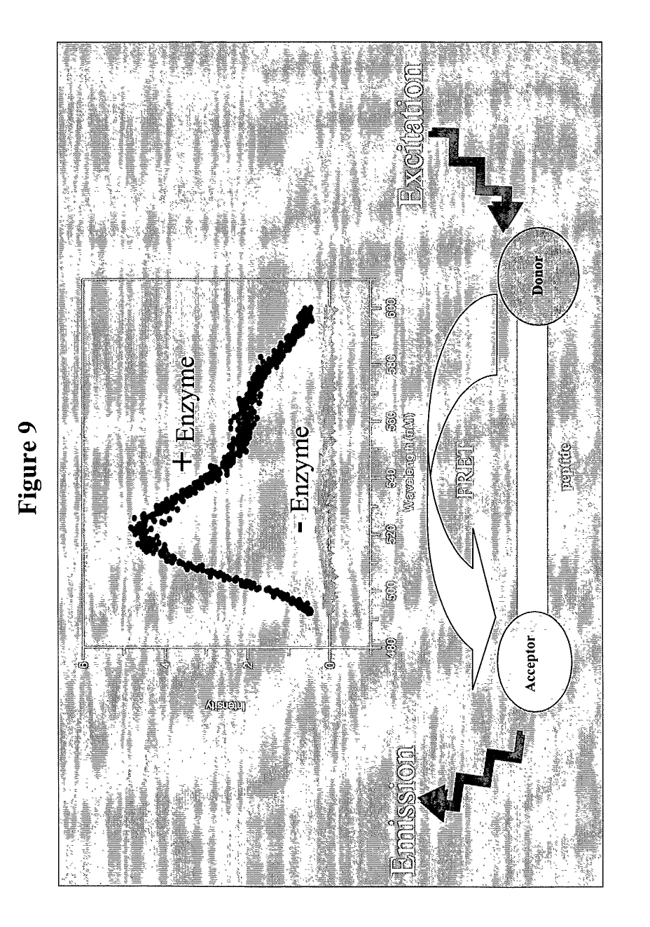

FRET is a process whereby a first fluorescent dye (the “donor” dye) is excited, typically by illumination, and transfers its absorbed energy to a second dye (the “acceptor” dye) that has a longer wavelength and therefore lower energy emission. Where the second dye is fluorescent, energy transfer results in fluorescence emission at the wavelength of the second dye. However, where the second dye is nonfluorescent, the absorbed energy does not result in fluorescence emission, and the fluorescence of the initial donor dye is said to be “quenched”. The general principle of FRET is illustrated in FIG. 9.

Energy transfer can also be utilised to quench the emission of luminescent donors, including phosphorescent and chemiluminescent donors. When a luminescent emission is restored by preventing energy transfer, the luminescence is said to be “dequenched” or “unquenched”. FRET has been utilised to study DNA hybridisation and amplification, the dynamics of protein folding, proteolytic degradation (as in the present invention), and interactions between other biomolecules. By far the most common donor-acceptor dye pair utilised for these applications is dabcyl (the quenching dye) and EDANS (the fluorophore) (as discussed in The Molecular Probes Handbook of Fluorescence Probes and Research Chemicals, 1996, chapter 9.3).

Despite the widespread use of the dabcyl-EDANS energy transfer pair, this technology possesses a number of shortcomings, including cellular autofluorescence, DNA cross-linking, and strong intrinsic absorptions of many drugs and biologically active proteins. Both dabcyl and EDANS have low extinction coefficients, resulting in assays that are comparatively insensitive.

In order to avoid the difficulties associated with the use of ultraviolet excitation, the absorption of the energy acceptor should be closely aligned with the visible light fluorophore used. Compounds have been discovered to quench the fluorescence of a large variety of dyes, including dyes that are excited in the ultraviolet, but also including fluoresceins, rhodamines, and even longer wavelength fluorophores such as CY 5 and allophycocyanin. In addition, such compounds have significantly larger extinction coefficients than the quenching compounds typically used in energy transfer assays.

An example of such a new and highly useful class of nonfluorescent energy acceptors is QSY™-7 (Molecular Probes, Inc., Oregon, USA). Preferably, said acceptor is paired in the FRET assays of the present invention with a donor fluorescent dye, preferably Rhodamine green. More preferably, said acceptor/donor FRET pairing is linked to a substrate peptide (which undergoes proteolytic degradation by e.g. SEP). Most preferably, said labelled substrate peptide is a small fluorogenic peptide, preferably Rhodamine green-Gly-Gly-dPhe-Leu-Arg-Arg-Val-Cys(QSY™7)-βAla-NH₂ (SEQ ID NO: 8).

SEP FRET Assays

Such FRET-based SEP assays are based on an assay developed by Carvalho et al. for use with NEP (Carvalho et al., Annal. Biochem. 237, pp. 167-173 (1996)). The SEP FRET assays utilise a similar intramolecularly quenched fluorogenic peptide substrate, but with a novel combination of fluorogenic donor/acceptor dyes.

The preparation of the synthetic labelled substrate peptides CP4, CP5, and CP6 are further described below.

Suitable buffers for use in human SEP assays can be any in which human SEP is found to be active and that does not otherwise interfere with the end result of the assays. Normally this will be a buffer that maintains a neutral pH. An example of such a buffer is 50 mM Tris Cl; pH 7.4. However, such a buffer is not preferred for use in a human SEP FRET assay as Tris is very prone to changes in pH due to temperature fluctuations. Therefore, a preferred buffer for use in a human SEP FRET assay is 50 mM HEPES; pH 7.4.

Peptidase FRET Assays

It will be understood by one of skill in the art that any of the above-mentioned novel peptide substrates, e.g., CP4, CP5, CP6, or others, may equally be used to analyse the proteolytic degradation of a wide variety of peptidase enzymes other than SEP.

Generally it is envisaged that the above-mentioned peptide substrates may be used in FRET assays to identify peptidase inhibitors (whereby the proteolytic action of the enzyme is inhibited by an inhibitor, thereby resulting in no signal (i.e. the fluorescence of the donor dye is “quenched”)).

Enzymes

The term “peptidase” can be used synonymously with the term “peptide hydrolase” for any enzyme that hydrolyses peptide bonds. Peptidases are further divided into “exopeptdases” that act only near a terminus of a polypeptide chain and “endopeptidases” that act internally in polypeptide chains. The types of exopeptidases and endopeptidases useful in the FRET assays of the present invention are described more fully below. The usage of the term “peptidase” is synonymous with the usage of the term “protease” as it was originally used (see Grassmann, W. and Dyckerhoff, H. Über die Proteinase und die Polypeptidase der Hefe. 13. Abhandlung uber Pflanzenproteasen in der von R. Willstätter und Mitarbeitem begonnenen Untersuchungsreihe. Hoppe-Seyler's Z. Physiol. Chem. 179 (1928) 41-78) as a general term for both exopeptidases and endopeptidases, but it should be noted that previously, in Enzyme Nomenclature (1984), “peptidase” was restricted to the enzymes included in sub-subclasses EC 3.4.11-19, the exopeptidases. Also, the term “proteinase” used previously for the enzymes included in sub-subclasses EC 3.4.21-99 carried the same meaning as “endopeptidase”, and has been replaced by “endopeptidase” for consistency.

Accordingly, the FRET assays of the present invention may also be used to identify inhibitors of peptidases (EC 3.4) generally, including exopeptidases (EC 3.4.11-19) and endopeptidases (EC 3.4.21-24 and EC 3.4.99).

Exopeptidases act only near the ends of polypeptide chains, and those acting at a free N-terminus liberate a single amino-acid residue (aminopeptidases, EC 3.4.11), or a dipeptide or a tripeptide (dipeptidyl-peptidases and tripeptidyl-peptidases, EC 3.4.14). Exopeptidases acting at a free C-terminus liberate a single residue (carboxypeptidases, EC 3.4.16-18) or a dipeptide (peptidyl-dipeptidases, EC 3.4.15). The carboxypeptidases are allocated to four groups on the basis of catalytic mechanism: the serine-type carboxypeptidases (EC 3.4.16), the metallocarboxypeptidases (EC 3.4.17) and the cysteine-type carboxypeptidases (EC 3.4.18). Other exopeptidases are specific for dipeptides (dipeptidases, EC 3.4.13), or remove terminal residues that are substituted, cyclized or linked by isopeptide bonds (peptide linkages other than those of a-carboxyl to a-amino groups) (omega peptidases, 3.4.19).

The sub-subclasses of exopeptidases mentioned above are all included within the scope of the range of enzymes useful in the FRET assays of the present invention. However, a preferred group of exopeptidases are aminopeptidases. An example of an aminopeptidase useful in the present invention is oxytocinase.

Endopeptidases are divided into sub-subclasses on the basis of catalytic mechanism, and specificity is used only to identify individual enzymes within the groups. These are the sub-subclasses of serine endopeptidases (EC 3.4.21), cysteine endopeptidases (EC 3.4.22), aspartic endopeptidases (EC 3.4.23), metalloendopeptidases (EC 3.4.24) and threonine endopeptidases (EC 3.4.25). Endopeptidases that are not assigned to any of the sub-subclasses EC 3.4.21-25 are listed in sub-subclass EC 3.4.99.

The sub-subclasses of endopeptidases mentioned above are all included within the scope of the range of enzymes useful in the FRET assays of the present invention. However, a preferred group of endopeptidases are metalloendopeptidases. Within the metalloendopeptidase group of enzymes is the neprilysin family of metalloendopeptidases. Examples of the neprilysin family of metalloendopeptidases include neprilysin (also called NEP, CD10, CALLA, enkephalinase or EC 3.4.24.11), endothelin-converting enzymes (ECE-1 and ECE-2), PEX, KELL, X-converting enzyme/damage induced neural endopeptidase (XCE/DINE), and an enzyme identified in rodents called soluble secreted endopeptidase/neprilysin II (SEP/NEPII; Ghaddar, G et al, Biochem Journal, Vol 347, 2000, pp. 419-429; Ikeda, K et al, Journal Biological Chemistry, Vol 274, 1999, pp. 32469-32477; Tanja, 0 et al, Biochem Biophys Research Communication, Vol 271, 2000, pp. 565-570; International Patent Application WO 99/53077). Preferred neprilysin family members are NEP and SEP. More preferred is SEP, including the human SEP described herein and in WO 02/06492.

In describing the specificity of endopeptidases, the term “oligopeptidase” is used to refer to those that act only (or optimally) on substrates smaller than proteins.

Whilst one embodiment of the present invention relates specifically to SEP FRET assays, also contemplated by the present invention are other peptidase FRET assays, preferably endopeptidase FRET assays, more preferably NEP FRET assays. Also contemplated by the present invention are oxytocinase FRET assays.

Assay Measurement

The method of measurement of the substrate and/or products in, for example, the human SEP assay will depend on the peptide substrate chosen and the nature of its modification. For example, if the substrate chosen contains a fluorescent group, a fluorimeter can be used. Similarly, if the substrate is radiolabelled, a scintillation counter may be used. Most substrates and products can be measured using high pressure liquid chromatography (HPLC) or mass spectrometry, and these would be the methods of choice if the substrate was not modified to include a radiolabel or fluorescence label.

Furthermore, the nucleotide sequence coding for human SEP or a sequence that is complementary thereto may also be used in assays to detect the presence of human SEP coding sequences in human cells. These assays would provide information regarding the tissue distribution of this enzyme and its biological relevance with respect to particular disease states.

The terms “SEP polypeptide”, “SEP enzyme” (and the like) encompass splice variants (isoenzymes) of the human SEP sequence. In particular, any one or more of the human SEP isoenzymes, the nucleotide sequences coding for same, the nucleotide sequences that are complementary to same, and the antibodies directed to same may be used in assays to screen for agents that selectively affect one of the isoenzymes. These assays would provide information regarding the biological relevance of each of the isoenzymes with respect to particular disease states. These assays would also allow one of skill in the art to test for and identify agents that are useful to affect the expression of or activity of human SEP—such as in a particular tissue or in a particular disease state.

### SEP POLYPEPTIDE

The term “polypeptide”—which is interchangeable with the term “protein”—includes single-chain polypeptide molecules as well as multiple-polypeptide complexes where individual constituent polypeptides are linked by covalent or non-covalent means.

Preferably, the SEP polypeptide described herein (and in detail in WO 02/06492, the content of which is incorporated herein by reference in its entirety) is a single-chain polypeptide.

The SEP polypeptide used in the methods of the present invention may be in a substantially isolated form. It will be understood that the polypeptide may be mixed with carriers or diluents which will not interfere with the intended purpose of the polypeptide and still be regarded as substantially isolated. The SEP polypeptide may also be in a substantially purified form, in which case it will generally comprise the polypeptide in a preparation in which more than 90%, e.g. 95%, 98% or 99% of the polypeptide in the preparation is a SEP polypeptide as described herein. SEP polypeptides may be modified for example by the addition of histidine residues to assist their purification.

The SEP polypeptides may also be produced by synthetic means (e.g. as described by Geysen et al., 1996) or recombinantly, as described below.

For the avoidance of doubt, it is contemplated that the methods of the invention may utilize both native (natural) and non-native forms of the SEP polypeptide (and the other peptidases mentioned herein as useful in the methods of the invention).

In a preferred embodiment, the amino acid sequence per se of the SEP polypeptide used in the methods of the present invention does not cover the native human SEP when it is in its natural environment and when it has been expressed by its native nucleotide coding sequence which is also in its natural environment and when that nucleotide sequence is under the control of its native promoter which is also in its natural environment. For ease of reference, we have called this preferred embodiment the “non-native amino acid sequence”.

The terms “variant”, “homologue”, “derivative” or “fragment” in relation to the amino acid sequence for the enzyme of the SEP polypeptide include any substitution of, variation of, modification of, replacement of, deletion of or addition of one (or more) amino acid from or to the sequence providing the resultant enzyme has SEP activity, preferably being at least as biologically active as the enzyme shown in attached SEQ ID NO: 2. In particular, the term “homologue” covers homology with respect to structure and/or function. With respect to sequence homology, preferably there is at least 78%, more preferably at least 85%, more preferably at least 90% homology to the sequence shown in SEQ ID NO: 2. More preferably there is at least 95%, more preferably at least 98%, homology to the sequence shown in SEQ ID NO: 2.

Typically, for the variant, homologue, derivative or fragment, the types of amino acid substitutions that could be made should maintain the hydrophobicity/hydrophilicity of the amino acid sequence. Amino acid substitutions may be made, for example from 1, 2 or 3 to 10, 20 or 30 substitutions provided that the modified sequence retains the ability to act as a SEP enzyme. Amino acid substitutions may include the use of non-naturally occurring analogues.

The amino acid sequence of the SEP polypeptide may be produced by expression of a nucleotide sequence coding for same in a suitable expression system.

In addition, or in the alternative, the protein itself could be produced using chemical methods to synthesise a human SEP amino acid sequence, in whole or in part. For example, peptides can be synthesised by solid phase techniques, cleaved from the resin, and purified by preparative high performance liquid chromatography (e.g. Creighton (1983) Proteins Structures and Molecular Principles, W H Freeman and Co., New York, N.Y., USA). The composition of the synthetic peptides may be confirmed by amino acid analysis or sequencing (e.g. the Edman degradation procedure).

Direct peptide synthesis can be performed using various solid-phase techniques (Roberge J Y et al, Science, Vol 269, 1995, pp. 202-204) and automated synthesis may be achieved, for example, using the ABI 431 A Peptide Synthesizer (Perkin Elmer, Boston, Mass., USA) in accordance with the instructions provided by the manufacturer. Additionally, the amino acid sequence of human SEP, or any part thereof, may be altered during direct synthesis and/or combined using chemical methods with a sequence from other subunits, or any part thereof, to produce a variant polypeptide.

In another embodiment, a human SEP natural, modified or recombinant sequence may be ligated to a heterologous sequence to encode a fusion protein. For example, for screening of peptide libraries for inhibitors of SEP activity, it may be useful to encode a chimeric SEP protein expressing a heterologous epitope that is recognised by a commercially available antibody. A fusion protein may also be engineered to contain a cleavage site located between a SEP sequence and the heterologous protein sequence, so that the SEP may be cleaved and purified away from the heterologous moiety.

Human SEP may also be expressed as a recombinant protein with one or more additional polypeptide domains added to facilitate protein purification. Such purification facilitating domains include, but are not limited to, metal chelating peptides such as histidine-tryptophan modules that allow purification on immobilised metals (Porath J, Protein Expr. Purif., Vol 3, 1992, pp. 263-281), protein A domains that allow purification on immobilised immunoglobulin, and the domain utilised in the FLAGS extension/affinity purification system (Immunex Corp, Seattle, Wash., USA). The inclusion of a cleavable linker sequence such as Factor XA or enterokinase (Invitrogen, San Diego, Calif., USA) between the purification domain and SEP is useful to facilitate purification.

A specific amino acid sequence of human SEP is shown in SEQ ID NO: 2. However, the present invention encompasses methods which use amino acid sequences encoding other members from the SEP family which would include amino acid sequences having at least 78% identity (more preferably at least 85% identity) to that specific amino acid sequence.

Polypeptides as described herein also include fragments of the present amino acid sequence and variants thereof. Suitable fragments will be at least 5, e.g. at least 10, 12, 15 or 20 amino acids in size.

Polypeptides as described herein may also be modified to contain one or more (e.g. at least 2, 3, 5 or 10) substitutions, deletions or insertions, including conserved substitutions. These aspects are discussed in a later section.

### SEP NUCLEOTIDE SEQUENCE

The term “nucleotide sequence” as used herein refers to an oligonucleotide sequence or polynucleotide sequence, and variants, homologues, fragments and derivatives thereof (such as portions thereof). The nucleotide sequence may be DNA or RNA which may be of genomic or synthetic or recombinant origin which may be double-stranded or single-stranded whether representing the sense or antisense strand.

Preferably, the term “nucleotide sequence” means DNA.

More preferably, the term “nucleotide sequence” means DNA prepared by use of recombinant DNA techniques (i.e. recombinant DNA).

For the avoidance of doubt, it is contemplated that the methods of the invention may utilize both native (natural) and non-native forms of the SEP polypeptide (and the other peptidases mentioned herein as useful in the methods of the invention) encoded by native (natural) or non-native (recombinant) nucleotide sequences, respectively.

In a preferred embodiment, the nucleotide sequence per se of the SEP polypeptide used in the methods of the present invention does not cover the native nucleotide coding sequence in its natural environment when it is under the control of its native promoter which is also in its natural environment. For ease of reference, we have called this preferred embodiment the “non-native nucleotide sequence”.

The nucleotide sequences described herein may include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the nucleotide sequences described herein may be modified by any method available in the art. Such modifications may be carried out in to enhance the in vivo activity or life span of nucleotide sequences.

The “SEP nucleotide” (“SEP polynucleotide”) also encompasses nucleotide sequences that are complementary to the sequences presented herein, or any derivative, fragment or variant thereof. If the sequence is complementary to a fragment thereof then that sequence can be used a probe to identify similar coding sequences in other organisms, etc.

The “SEP nucleotide” (“SEP polynucleotide”) also encompasses nucleotide sequences that are capable of hybridising to the sequences presented herein, or any derivative, fragment or variant thereof.

The “SEP nucleotide” (“SEP polynucleotide”) also encompasses nucleotide sequences that are capable of hybridising to the sequences that are complementary to the sequences presented herein, or any derivative, fragment or variant thereof.

The term “variant” also encompasses sequences that are complementary to sequences that are capable of hybridising to the nucleotide sequences presented herein.

Preferably, the term “variant” encompasses sequences that are complementary to sequences that are capable of hybridising under stringent conditions (e.g. 65° C. and 0.1×SSC {1×SSC=0.15 M NaCl, 0.015 Na₃ citrate pH 7.0}) to the nucleotide sequences presented herein.

The “SEP nucleotide” (“SEP polynucleotide”) also relates to nucleotide sequences that can hybridise to the nucleotide sequences presented herein (including complementary sequences of those presented herein).

The term “SEP nucleotide” (“SEP polynucleotide”) also relates to nucleotide sequences that are complementary to sequences that can hybridise to the nucleotide sequences presented herein (including complementary sequences of those presented herein).

The term “SEP nucleotide” (“SEP polynucleotide”) also relates to nucleotide sequences that are capable of hybridising to the nucleotide sequences presented herein under conditions of intermediate to maximal stringency.

The term “SEP nucleotide” (“SEP polynucleotide”) also relates to nucleotide sequences that can hybridise to the nucleotide sequence presented herein, or the complement thereof, under stringent conditions (e.g. 65° C. and 0.1×SSC).

Exemplary nucleic acids can alternatively be characterised as those nucleotide sequences which encode a human SEP protein and hybridise to the DNA sequence shown in SEQ ID NO: 1 or SEQ ID NO: 5. Preferred are such sequences encoding human SEP which hybridise under high-stringency conditions to the sequence shown in SEQ ID NO: 1 or SEQ ID NO: 5 or the complement thereof.

Advantageously, there are provided nucleic acid sequences which are capable of hybridising, under stringent conditions, to a fragment of the sequence shown in the SEQ ID NO: 1 or SEQ ID NO: 5 or the complement thereof. Preferably, the fragment is between 15 and 50 bases in length. Advantageously, it is about 25 bases in length.

The terms “variant”, “homologue”, “derivative” or “fragment” in relation to the nucleotide sequence coding for the preferred enzyme used in the methods of the present invention include any substitution of, variation of, modification of, replacement of, deletion of or addition of one (or more) nucleic acid from or to the sequence providing the resultant nucleotide sequence codes for or is capable of coding for an enzyme having SEP activity, preferably being at least as biologically active as the enzyme encoded by the sequence shown in SEQ ID NO: 1 or SEQ ID NO: 5. In particular, the term “homologue” covers homology with respect to structure and/or function providing the resultant nucleotide sequence codes for or is capable of coding for an enzyme having SEP activity. With respect to sequence homology, preferably there is at least 83%, more preferably at least 85%, more preferably at least 90% homology to a nucleotide sequence coding for the amino acid sequence shown in SEQ ID NO: 2. More preferably there is at least 95%, more preferably at least 98% homology to a nucleotide sequence coding for the amino acid sequence shown in SEQ ID NO: 2. With respect to sequence homology, preferably there is at least 83%, more preferably at least 85%, more preferably at least 90% homology to the sequence shown in SEQ ID NO: 1 or SEQ ID NO: 5. More preferably there is at least 95%, more preferably at least 98%, homology to the sequence shown in SEQ ID NO: 1 or SEQ ID NO: 5.

As indicated, the present invention preferably relates to methods using human SEP (SEP polypeptide) encoded, preferably, by a cDNA sequence.

The present invention also relates to methods using human SEP (SEP polypeptide) encoded by (i) DNA segments comprising the DNA sequence shown in SEQ ID NO: 1 or SEQ ID NO: 5 or allelic variations thereof, (ii) non-native DNA comprising the DNA sequence shown in SEQ ID NO: 1 or SEQ ID NO: 5 or allelic variations thereof, or (iii) recombinant DNA comprising the DNA sequence shown in SEQ ID NO: 1 or SEQ ID NO: 5 or allelic variations thereof.

Polynucleotides as described herein include nucleic acid sequences encoding the polypeptides as used in the methods of the present invention. It will be appreciated that a range of different polynucleotides encode a given amino acid sequence as a consequence of the degeneracy of the genetic code.

By knowledge of the amino acid sequences set out herein it is possible to devise partial and full-length nucleic acid sequences such as cDNA and/or genomic clones that encode the polypeptides as described herein. For example, polynucleotides may be obtained using degenerate polymerase chain reaction (PCR), which will use primers designed to target sequences encoding the amino acid sequences presented herein. The primers will typically contain multiple degenerate positions. However, to minimise degeneracy, sequences will be chosen that encode regions of the amino acid sequences presented herein containing amino acids such as methionine, which are coded for by only one triplet. In addition, sequences will be chosen to take into account codon usage in the organism whose nucleic acid is used as the template DNA for the PCR procedure. PCR will be used at stringency conditions lower than those used for cloning sequences with single sequence (non-degenerate) primers against known sequences.

Nucleic acid sequences obtained by PCR that encode polypeptide fragments used in the methods of the present invention may then be used to obtain larger sequences using hybridisation library screening techniques. For example a PCR clone may be labelled with radioactive atoms and used to screen a cDNA or genomic library from other species, preferably other mammalian species. Hybridisation conditions will typically be conditions of medium to high stringency (for example 0.03M sodium chloride and 0.03M sodium citrate at from about 50° C. to about 60° C.).

Degenerate nucleic acid probes encoding all or part of the amino acid sequence may also be used to probe cDNA and/or genomic libraries from other species, preferably other mammalian species. However, it is preferred to carry out PCR techniques initially to obtain a single sequence for use in further screening procedures.

Polynucleotide sequences which encode human SEP, fragments of the polypeptide, fusion proteins or functional equivalents thereof, may be used to generate recombinant DNA molecules that direct the expression of human SEP in appropriate host cells. Due to the inherent degeneracy of the genetic code, other DNA sequences, which encode substantially the same, or a functionally equivalent amino acid sequence, may be used to clone and express human SEP. As will be understood by those of skill in the art, it may be advantageous to produce human SEP-encoding nucleotide sequences possessing non-naturally occurring codons. Codons preferred by a particular prokaryotic or eukaryotic host (Murray E et al, (1989), Nuc. Acids Res., 17:477-508) can be selected, for example, to increase the rate of human SEP expression or to produce recombinant RNA transcripts having desirable properties, such as a longer half-life, than transcripts produced from naturally occurring sequence.

Polynucleotide sequences described herein, obtained using the techniques described above, may be used to obtain further homologous sequences and variants using the techniques described above. They may also be modified for use in expressing the polypeptides in a variety of host cells systems, for example to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

Altered human SEP polynucleotide sequences which may be used in accordance with the invention include deletions, insertions or substitutions of different nucleotide residues resulting in a polynucleotide that encodes the same or a functionally equivalent SEP. The protein may also have deletions, insertions or substitutions of amino acid residues, which produce a silent change and result in a functionally equivalent SEP. Deliberate amino acid substitutions may be made on the basis of similarity in polarity, charge, solubility, hydrophobicity, hydrophilicity, and/or the amphipathic nature of the residues as long as the biological activity of SEP is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, senne, threonine, phenylalanine, and tyrosine.

Included within the scope of the description are alleles of human SEP. As used herein, an “allele” or “allelic sequence” is an alternative form of human SEP. Alleles result from a mutation, i.e. a change in the nucleic acid sequence, and generally produce altered mRNAs or polypeptides whose structure or function may or may not be altered. Any given gene may have none, one or many allelic forms. Common mutational changes, which give rise to alleles, are generally ascribed to deletions, additions or substitutions of amino acids. Each of these types of changes may occur alone, or in combination with the others, one or more times in a given sequence.

The nucleotide sequences described herein may be engineered in order to alter a human SEP coding sequence for a variety of reasons, including but not limited to, alterations which modify the cloning, processing and/or expression of the gene product. For example, mutations may be introduced using techniques, which are well known in the art, e.g. site-directed mutagenesis to insert new restriction sites, to alter glycosylation patterns or to change codon preference.

Polynucleotides as described herein may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides as used herein.

Polynucleotides or primers as described herein may carry a revealing label. Suitable labels include radioisotopes such as ³²P or ³⁵S, enzyme labels, or other protein labels such as biotin. Such labels may be added to polynucleotides or primers and may be detected using by techniques known in the art.

Polynucleotides such as a DNA polynucleotide and primers may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using PCR cloning techniques. This will involve making a pair of primers (e.g. of about 15-30 nucleotides) to a region of the nucleotide sequence which it is desired to clone, bringing the primers into contact with mRNA or cDNA obtained from, e.g., a fungal, plant or prokaryotic cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

DNA molecules may be modified to increase intracellular stability and half-life. Possible modifications include, but are not limited to, the addition of flanking sequences of the 5′ and/or 3′ ends of the molecule or the use of phosphorothioate or 2′ O-methyl rather than phosphodiesterase linkages within the backbone of the molecule.

Furthermore, DNA sequences, once known, give the information needed to design assays to specifically detect isoenzymes or splice variants. Isoenzyme-specific PCR primer pairs are but one example of an assay that depends completely on the knowledge of the specific DNA sequence of the isoenzyme or splice variant. Such an assay allows detection of mRNA for the isoenzyme to access the tissue distribution and biological relevance of each isoenzyme to a particular disease state. It also allows identification of cell lines that may naturally express only one isoenzyme—a discovery that might obviate the need to express recombinant genes. If specific human SEP isoenzymes are shown to be associated with a particular disease state, this would be valuable in the design of diagnostic assays to detect the presence of isoenzyme MRNA.

An abnormal level of nucleotide sequences encoding a human SEP enzyme in a biological sample may reflect a chromosomal aberration, such as a nucleic acid deletion or mutation. Accordingly, nucleotide sequences encoding a human SEP enzyme provide the basis for probes, which can be used diagnostically to detect chromosomal aberrations such as deletions, mutations or chromosomal translocations in the gene encoding human SEP. Human SEP gene expression may be altered in such disease states or there may be a chromosomal aberration present in the region of the gene encoding a human SEP.

In an alternative embodiment, the coding sequence of human SEP could be synthesised, in whole or in part, using chemical methods well known in the art (see Caruthers M H et al, (1980), Nuc. Acids Res. Symp. Ser., pp. 215-223; Horn T et al, (1980), Nuc. Acids Res. Symp. Ser., pp. 225-232).

### NATURALLY OCCURRING

As used herein “naturally occurring” refers to a human SEP with an amino acid sequence found in nature.

### ISOLATED/PURIFIED

As used herein, the terms “isolated” and “purified” refer to molecules, either nucleic or amino acid sequences, that are removed from their natural environment and isolated or separated from at least one other component with which they are naturally associated.

### BIOLOGICALLY ACTIVE

As used herein “biologically active” refers to a protein (especially enzyme)—such as a human SEP (SEP polypeptide), preferably recombinant human SEP—having a similar structural function (but not necessarily to the same degree), and/or similar regulatory function (but not necessarily to the same degree), and/or similar biochemical function (but not necessarily to the same degree) and/or immunological activity (but not necessarily to the same degree) of the naturally occurring protein (especially enzyme). Specifically, a human SEP has the ability to proteolytically cleave certain peptide substrates, which is one of the characteristic activities of the human SEP enzyme presented herein.

### IMMUNOLOGICAL ACTIVITY

As used herein, “imnunological activity” is defined as the capability of a natural, recombinant or synthetic protein (especially enzyme) or any oligopeptide thereof, to induce a specific immune response in appropriate animals or cells and to bind with specific antibodies.

### DERIVATIVE

The term “derivative” as used herein in relation to the amino acid sequence includes chemical modification of a human SEP. Illustrative of such modifications would be replacement of hydrogen by an alkyl, acyl, or amino group.

### DELETION

As used herein a “deletion” is defined as a change in either nucleotide or amino acid sequence in which one or more nucleotides or amino acid residues, respectively, are absent.

### INSERTION/ADDITION

As used herein an “insertion” or “addition” is a change in a nucleotide or amino acid sequence, which has resulted in the addition of one or more nucleotides or amino acid residues, respectively, as compared to the naturally occurring human SEP.

### SUBSTITUTION

As used herein “substitution” results from the replacement of one or more nucleotides or amino acids by different nucleotides or amino acids, respectively.

### HOMOLOGUE

The term “homologue” with respect to the nucleotide sequences and the amino acid sequences described herein may be synonymous with allelic variations of the sequences.

In particular, the term “homology” as used herein may be equated with the term “identity”. Here, sequence homology with respect to the nucleotide sequence and the amino acid sequence described herein can be determined by a simple “eyeball” comparison (i.e. a strict comparison) of any one or more of the sequences with another sequence to see if that other sequence has at least 83% identity to the nucleotide sequence and at least 78% identity to the amino acid sequences. Relative sequence homology (i.e. sequence identity) can also be determined by commercially available computer programs that can calculate percentage (%) homology between two or more sequences. Typical examples of such computer programs are CLUSTAL or BLAST.

Percentage (%) homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software that can perform sequence comparisons include, but are not limited to, the BLAST package (see Ausubel et al., 1999 ibid—Chapter 18), FASTA (Altschul et al., 1990, J. Mol. Biol., 403-410) and the GENEWORKS suite of comparison tools. Both BLAST and FASTA are available for off-line and on-line searching (see Ausubel et al., 1999 ibid, pages 7-58 to 7-60). However, for some applications it is preferred to use the GCG Bestfit program.

Although the final % homology can be measured in terms of identity, in some cases, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

As indicated, for some applications, sequence homology (or identity) may be determined using any suitable homology algorithm, using for example default parameters. For a discussion of basic issues in similarity searching of sequence databases, see Altschul et al. (1994) Nature Genetics 6:119-129. For some applications, the BLAST algorithm is employed, with parameters set to default values. The BLAST algorithm is described in detail at http://www.ncbi.nih.gov/BLAST/blast_help.html. Advantageously, “substantial homology” when assessed by BLAST equates to sequences which match with an EXPECT value of at least about e-7, preferably at least about e-9 and most preferably e-10 or lower. The default threshold for EXPECT in BLAST searching is usually 10.

Should Gap Penalties be used when determining sequence identity, then preferably the following parameters are used:

| FOR BLAST | |
|---|---|
| GAP OPEN | 5 |
| GAP EXTENSION | 2 |

| FOR CLUSTAL | DNA | PROTEIN | |
|---|---|---|---|
| WORD SIZE | 2 | 1 | K triple |
| GAP PENALTY | 10 | 10 | |
| GAP EXTENSION | 0.1 | 0.1 | |

Other computer program methods to determine identify and similarity between the two sequences include but are not limited to the GCG program package (Devereux et al., 1984, Nucleic Acids Research, 12: 387) and FASTA (Altschul et al, 1990, J. Molec. Biol., pp. 403-410).

### POLYPEPTIDE VARIANTS AND DERIVATIVES

The terms “variant” or “derivative” in relation to the amino acid sequences described herein includes any substitution of, variation of, modification of, replacement of, deletion of or addition of one (or more) amino acids from or to the sequence providing the resultant amino acid sequence has human SEP activity, preferably having at least the same activity as the polypeptide presented in SEQ ID NO: 2.

The sequences presented herein may be modified for use in the methods of the present invention. Typically, modifications are made that maintain the human SEP activity of the sequence. Amino acid substitutions may be made, for example from 1, 2 or 3 to 10, 20 or 30 substitutions provided that the modified sequence retains the human SEP activity. Amino acid substitutions may include the use of non-naturally occurring analogues.

Conservative substitutions may be made, for example, according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

As indicated above, proteins as presented herein are typically made by recombinant means and/or by using synthetic means using techniques well known to the skilled person such as solid phase synthesis. Variants and derivatives of such sequences include fusion proteins, wherein the fusion proteins comprise at least the amino acid sequence of the SEP polypeptide described herein being linked (directly or indirectly) to another amino acid sequence. These other amino acid sequences—which are sometimes referred to as fusion protein partners—will typically impart a favourable functionality—such as to aid extraction and purification of the amino acid sequence. Examples of fusion protein partners include glutathione-S-transferase (GST), 6xHis, GAL4 (DNA binding and/or transcriptional activation domains) and β-galactosidase. It may also be convenient to include a proteolytic cleavage site between the fusion protein partner and the protein sequence so as to allow removal of the latter. Preferably the fusion protein partner will not hinder the function of the protein.

### POLYNUCLEOTIDE VARIANTS AND DERIVATIVES

The terms “variant” or “derivative” in relation to the nucleotide sequence described herein include any substitution of, variation of, modification of, replacement of, deletion of or addition of one (or more) nucleic acid from or to the sequence providing the resultant nucleotide sequence codes for a polypeptide having human SEP activity, preferably having at least the same activity as the polypeptide encoded by the sequence presented in SEQ ID NO: 1 or SEQ ID NO: 5.

As indicated above, with respect to sequence homology, preferably there is at least 83%, more preferably at least 85%, more preferably at least 90% homology to the sequence shown in SEQ ID NO: 1 or SEQ ID NO: 5. More preferably there is at least 95%, more preferably at least 98%, homology. Nucleotide homology comparisons may be conducted as described above. For some applications, a preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

As used herein, the terms “variant”, “homologue”, “fragment” and “derivative” embrace allelic variations of the sequences.

The term “variant” also encompasses sequences that are complementary to sequences that are capable of hybridising to the nucleotide sequences presented herein.

### HYBRIDISATION

The term “hybridisation” as used herein shall include “the process by which a strand of nucleic acid joins with a complementary strand through base pairing” (Coombs J (1994) Dictionary of Biotechnology, Stockton Press, New York N.Y.) as well as the process of amplification as carried out in PCR technologies as described in Dieffenbach C W and G S Dveksler (1995, PCR Primer, a Laboratory Manual, Cold Spring Harbor Press, Plainview, N.Y., USA).

Hybridisation conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego, Calif., USA), and confer a defined “stringency” as explained below.

Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid, which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency refers to conditions that permit hybridisation of only those nucleic acid sequences that form stable hybrids in 1 M Na⁺ at 65-68° C.

Maximum stringency typically occurs at about Tm-5° C. (5° C. below the Tm of the probe).

High stringency occurs at about 5° C. to 10° C. below the Tm of the probe. High stringency conditions can be provided, for example, by hybridisation in an aqueous solution containing 6×SSC, 5×Denhardt's, 1% SDS (sodium dodecyl sulphate), 0.1 Na⁺ pyrophosphate and 0.1 mg/ml denatured salmon sperm DNA as non specific competitor. Following hybridisation, high stringency washing may be done in several steps, with a final wash (about 30 min) at the hybridisation temperature in 0.2-0.1×SSC, 0.1% SDS.

Moderate, or intermediate, stringency typically occurs at about 10° C. to 20° C. below the Tm of the probe.

Low stringency typically occurs at about 20° C. to 25° C. below the Tm of the probe.

As will be understood by those of skill in the art, a maximum stringency hybridisation can be used to identify or detect identical polynucleotide sequences while an intermediate (or low) stringency hybridisation can be used to identify or detect similar or related polynucleotide sequences.

Moderate stringency refers to conditions equivalent to hybridisation in the above-described solution but at about 60-62° C. In that case the final wash is performed at the hybridisation temperature in 1×SSC, 0.1% SDS.

Low stringency refers to conditions equivalent to hybridisation in the above-described solution at about 50-52° C. In that case, the final wash is performed at the hybridisation temperature in 2×SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g., Sambrook, et al., eds. (1989) Molecular Cloning: A Laboratory Manual, Cold Spring Harbor Laboratory Press, New York; or Ausubel, et al., eds. (1990) Current Protocols in Molecular Biology, John Wiley & Sons, Inc.). Optimal hybridisation conditions have to be determined empirically, as the length and the GC content of the probe also play a role.

Polynucleotides capable of selectively hybridising to the nucleotide sequences presented herein, or to their complement, will be generally at least 83%, preferably at least 85% or 90% and more preferably at least 95% or 98% homologous to the corresponding nucleotide sequences presented herein over a region of at least 20, preferably at least 25 or 30, for instance at least 40, 60 or 100 or more contiguous nucleotides.

The term “selectively hybridisable” means that the polynucleotide used as a probe is used under conditions where a target polynucleotide is found to hybridize to the probe at a level significantly above background. The background hybridization may occur because of other polynucleotides present, for example, in the cDNA or genomic DNA library being screened. In this event, background implies a level of signal generated by interaction between the probe and a non-specific DNA member of the library which is less than 10-fold, preferably less than 100-fold as intense as the specific interaction observed with the target DNA. The intensity of interaction may be measured, for example, by radiolabelling the probe, e.g. with ³²P.

Where the polynucleotide is double-stranded, both strands of the duplex, either individually or in combination, are encompassed by the term “polynucleotide”. Where the polynucleotide is single-stranded, it is to be understood that the complementary sequence of that polynucleotide is also included within the scope of the term “polynucleotide”.

Other variants of the sequences described herein may be obtained, for example, by probing DNA libraries made from a range of individuals, for example individuals from different populations. In addition, other viral/bacterial, or cellular homologues particularly cellular homologues found in mammalian cells (e.g. bovine, ovine, porcine, equine and primate cells), may be obtained and such homologues and fragments thereof in general will be capable of selectively hybridising to the sequence shown in SEQ ID NO: 1 or SEQ ID NO: 5. Such sequences may be obtained by probing cDNA libraries made from, or genomic DNA libraries derived from, other animal species, and probing such libraries with probes comprising all or part of the sequence shown in SEQ ID NO: 1 or SEQ ID NO: 5 under conditions of medium to high stringency. Similar considerations apply to obtaining species homologues and allelic variants of the polypeptide or nucleotide sequences of the invention.

Variants and strain/species homologues may also be obtained using degenerate PCR, which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site-directed mutagenesis of characterised sequences. This may be useful where, for example, silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

The polynucleotides as described herein may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term “polynucleotides” as used herein.

Polynucleotides such as a DNA polynucleotides and probes may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR cloning techniques. This will involve making a pair of primers (e.g. of about 15 to 30 nucleotides) flanking a region of the sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

The following list represents titles of descriptive text, which is reproduced under the same (or similar) titles in WO 02/06492, the content of which is incorporated herein by reference in its entirety: Regulatory sequences, Secretion, Constructs, Vectors, Tissue, Host cells, Organism, Transformation of host cells/host organisms, Genetically engineered or genetically modified, Functionally disrupted, Genetically modified animal cell, Production of the polypeptide, Ribozymes, Detection, Diagnostics, Probes, Pharmaceuticals and Pharmaceutical combinations.

### ANTIBODIES

The amino acid sequence of the SEP polypeptide described herein can also be used to generate antibodies—such as by use of standard techniques—against the amino acid sequence.

Procedures well known in the art may be used for the production of antibodies to human SEP polypeptides. Such antibodies include, but are not limited to, polyclonal, monoclonal, chimeric, single chain, Fab fragments and fragments produced by a Fab expression library. Neutralising antibodies, i.e. those which inhibit biological activity of human SEP polypeptides, are especially preferred for diagnostics and therapeutics.

For the production of antibodies, various hosts including goats, rabbits, rats, mice, etc. may be immunised by injection with the peptidase (e.g. human SEP) or any portion, variant, homologue, fragment or derivative thereof or oligopeptide which retains immunogenic properties. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed.

Monoclonal antibodies to the peptidase (e.g. human SEP) may be even prepared using any technique which provides for the production of antibody molecules by continuous cell lines in culture. These include, but are not limited to, the hybridoma technique originally described by Kohler and Milstein (1975, Nature, Vol 256, pp. 495-497), the human B-cell hybridoma technique (Kosbor et al., (1983), Immunol. Today, Vol 4, p. 72; Cote et al., (1983), Proceedings of the National Academy of Sciences (USA), Vol 80, pp. 2026-2030) and the EBV-hybridoma technique (Cole et al., (1985), Monoclonal Antibodies and Cancer Therapy, Alan R Liss Inc., pp. 77-96). In addition, techniques developed for the production of “chimeric antibodies”, the splicing of mouse antibody genes to human antibody genes to obtain a molecule with appropriate antigen specificity and biological activity can be used (Morrison et al., (1984), Proceedings of the National Academy of Sciences (USA), Vol 81, pp. 6851-6855; Neuberger et al., (1984), Nature, Vol 312, pp. 604-608; Takeda et al., (1985), Nature, Vol 314, pp. 452-454). Alternatively, techniques described for the production of single chain antibodies (U.S. Pat. No. 4,946,779) can be adapted to produce inhibitor-specific single chain antibodies.

Antibodies may also be produced by inducing in vivo production in the lymphocyte population or by screening recombinant immunoglobulin libraries or panels of highly specific binding reagents as disclosed in Orlandi et al. (1989, Proceedings of the National Academy of Sciences (USA), Vol 86, pp. 3833-3837), and Winter G and Milstein C (1991; Nature, Vol 349, pp. 293-299).

Antibody fragments, which contain specific binding sites for human SEP, may also be generated. For example, such fragments include, but are not limited to, the F(ab′)₂ fragments which can be produced by pepsin digestion of the antibody molecule and the Fab fragments which can be generated by reducing the disulphide bridges of the F(ab′)₂ fragments. Alternatively, Fab expression libraries may be constructed to allow rapid and easy identification of monoclonal Fab fragments with the desired specificity (Huse W D et al., (1989), Science, Vol 256, pp.1275-1281).

An alternative technique involves screening phage display libraries where, for example, the phage express scFv fragments on the surface of their coat with a large variety of complementarity determining regions (CDRs). This technique is well known in the art.

Human SEP-specific antibodies are useful for the diagnosis of conditions and diseases associated with expression of human SEP polypeptide. A variety of protocols for competitive binding or immunoradiometric assays using either polyclonal or monoclonal antibodies with established specificities are well known in the art. Such immunoassays typically involve the formation of complexes between human SEP polypeptides and its specific antibody (or similar human SEP-binding molecule) and the measurement of complex formation. A two-site, monoclonal-based immunoassay utilizing monoclonal antibodies reactive to two non-interfering epitopes on a specific human SEP protein is preferred, but a competitive binding assay may also be employed. These assays are described in Maddox D E et al. (1983, Journal of Experimental Medicine, Vol 158, p.1211).

Anti-human SEP antibodies are useful for the diagnosis of disorders involving abnormal peptide signalling or other disorders or diseases characterised by abnormal expression of a human SEP. Diagnostic assays for a human SEP include methods utilising the antibody and a label to detect a human SEP polypeptide in human body fluids, cells, tissues or sections or extracts of such tissues. The polypeptides and antibodies described herein may be used with or without modification. Frequently, the polypeptides and antibodies will be labelled by joining them, either covalently or non-covalently, with a reporter molecule. A wide variety of reporter molecules are known to those of skill in the art.

Antibodies may be used to detect polypeptides as described herein present in biological samples by a method which comprises: (a) providing an antibody as described hereinbefore; (b) incubating a biological sample with said antibody under conditions which allow for the formation of an antibody-antigen complex; and (c) determining whether antibody-antigen complex comprising said antibody is formed.

Depending on the circumstances, suitable samples may include extract tissues such as testis or brain or from neoplastic growths derived from such tissues.

Antibodies may be bound to a solid support and/or packaged into kits in a suitable container along with suitable reagents, controls, instructions and the like.

### ASSAYS/IDENTIFICATION METHODS

The application also describes an assay method for detecting the presence of human SEP in cells (such as human cells) comprising: (a) performing a reverse transcriptase-polymerase chain reaction (RT-PCR) on RNA (such as total RNA) from such cells using a pair of PCR primers that are specific for human SEP, as determined from the DNA sequence shown in SEQ ID NO: 1 or SEQ ID NO: 5 or an allelic variation thereof; and (b) assaying the appearance of an appropriately sized PCR fragment - such as by agarose gel electrophoresis.

The present invention also relates to a method of identifying agents (such as compounds, other substances or compositions comprising the same) that affect (such as inhibit or otherwise modify) the activity of human SEP and/or the expression thereof, the method comprising contacting human SEP or the nucleotide sequence coding for the same with the agent and then measuring the activity of human SEP and/or the expression thereof.

The present invention also relates to a method of identifying agents (such as compounds, other substances or compositions comprising the same) that selectively affect (such as selectively inhibit or otherwise selectively modify) the activity of human SEP and/or the expression thereof, the method comprising contacting human SEP or the nucleotide sequence coding for the same with the agent and then measuring the activity of human SEP and/or the expression thereof.

The present invention also relates to a method of identifying agents (such as compounds, other substances or compositions comprising the same) that affect (such as inhibit or otherwise modify) the activity of human SEP and/or the expression thereof, the method comprising measuring the activity of human SEP and/or the expression thereof in the presence of the agent or after the addition of the agent in: (a) a cell line into which has been incorporated recombinant DNA comprising the DNA sequence shown in SEQ ID NO: 1 or SEQ ID NO: 5 or an allelic variation thereof, or (b) a cell population or cell line that naturally selectively expresses human SEP. Preferably, the activity of human SEP is determined by the assay method described above.

The present invention also relates to a method of identifying agents (such as compounds, other substances or compositions comprising the same) that selectively affect (such as inhibit or otherwise modify) the activity of human SEP and/or the expression thereof, the method comprising measuring the activity of human SEP and/or the expression thereof in the presence of the agent or after the addition of the agent in: (a) a cell line into which has been incorporated recombinant DNA comprising the DNA sequence shown in SEQ ID NO: I or SEQ ID NO: 5 or an allelic variation thereof, or (b) a cell population or cell line that naturally selectively expresses human SEP. Preferably, the activity of human SEP is determined by the assay method described above.

The present invention also relates to a method of screening an agent for modulation (preferably for specific modulation) of human SEP (or a derivative, homologue, variant or fragment thereof) activity or the expression of the nucleotide sequence coding for the same (including a derivative, homologue, variant or fragment thereof), the method comprising the steps of: (a) providing a candidate agent; (b) combining human SEP (or the derivative, homologue, variant or fragment thereof) or the nucleotide sequence coding for the same (or the derivative, homologue, variant or fragment thereof) with the candidate agent for a time sufficient to allow modulation under suitable conditions; and (c) detecting modulation of human SEP by the candidate agent (or the derivative, homologue, variant or fragment thereof) or the nucleotide sequence coding for the same (or the derivative, homologue, variant or fragment thereof) in order to ascertain if the candidate agent modulates human SEP (or the derivative, homologue, variant or fragment thereof) activity or the expression of the nucleotide sequence coding for the same (or the derivative, homologue, variant or fragment thereof).

The present invention also relates to a method of screening an agent for specific binding affinity with human SEP (or a derivative, homologue, variant or fragment thereof) or the nucleotide sequence coding for the same (including a derivative, homologue, variant or fragment thereof), the method comprising the steps of: (a) providing a candidate agent; (b) combining human SEP (or the derivative, homologue, variant or fragment thereof) or the nucleotide sequence coding for the same (or the derivative, homologue, variant or fragment thereof) with the candidate agent for a time sufficient to allow binding under suitable conditions; and (c) detecting binding of the candidate agent to human SEP (or the derivative, homologue, variant or fragment thereof) or the nucleotide sequence coding for the same (or the derivative, homologue, variant or fragment thereof) in order to ascertain if the candidate agent binds to human SEP (or the derivative, homologue, variant or fragment thereof) or the nucleotide sequence coding for the same (or the derivative, homologue, variant or fragment thereof).

The present invention also relates to a method of identifying an agent which is capable of modulating human SEP, the method comprising the steps of: (a) contacting the agent with human SEP (or a derivative, homologue, variant or fragment thereof) or the nucleotide sequence coding for the same (or the derivative, homologue, variant or fragment thereof); (b) incubating the mixture of step (a) with a bioactive peptide under conditions suitable for the proteolysis of the bioactive peptide; (c) measuring the amount of bioactive peptide proteolysis; and (d) comparing the amount of bioactive peptide proteolysis of step (c) with the amount of bioactive peptide proteolysis obtained with human SEP (or the derivative, homologue, variant or fragment thereof) or the nucleotide sequence coding for the same (or the derivative, homologue, variant or fragment thereof) incubated without the agent, thereby determining whether the agent affects (such as inhibits or selectively inhibits) bioactive peptide proteolysis.

Thus, in certain embodiments of the present invention, human SEP or a variant, homologue, fragment or derivative thereof and/or a cell line that expresses the human SEP or variant, homologue, fragment or derivative thereof may be used to screen for antibodies, peptides, or other agents, such as organic or inorganic molecules, that act as modulators of endopeptidase activity or for the expression thereof, thereby identifying a therapeutic agent capable of modulating bioactive peptide levels. For example, anti-human SEP antibodies capable of neutralising the activity of human SEP may be used to inhibit human SEP proteolysis of bioactive peptides, thereby increasing their levels. Alternatively, screening of peptide libraries or organic libraries made by combinatorial chemistry with recombinantly expressed human SEP or a variant, homologue, fragment or derivative thereof or cell lines expressing human SEP or a variant, homologue, fragment or derivative thereof may be useful for identification of therapeutic agents that function by modulating (e.g. inhibiting or selectively inhibiting) human SEP proteolysis of bioactive peptides. Synthetic compounds, natural products, and other sources of potentially biologically active materials can be screened in a number of ways deemed to be routine to those of skill in the art. For example, nucleotide sequences encoding the N-terminal region of human SEP may be expressed in a cell line, which can be used for screening of allosteric modulators, either agonists or antagonists, of human SEP activity. Alternatively, nucleotide sequences encoding the conserved catalytic domain of human SEP can be expressed in cell lines and used to screen for inhibitors or selective inhibitors of bioactive peptide proteolysis.

The ability of a test agent to interfere with human SEP activity or bioactive peptide proteolysis may be determined by measuring human SEP levels or bioactive peptide levels.

Accordingly, the present invention relates to a method of identifying a compound which is capable of modulating the bioactive peptide proteolysis activity of a human SEP, or a variant, homologue, fragment or derivative thereof, comprising the steps of (a) contacting the compound with a human SEP, or a variant, homologue, fragment or derivative thereof; (b) incubating the mixture of step (a) with a bioactive peptide under conditions suitable for the proteolysis of the bioactive peptide; (c) measuring the amount of bioactive peptide proteolysis; and (d) comparing the amount of bioactive peptide proteolysis of step (c) with the amount of bioactive peptide proteolysis obtained with the human SEP, or a variant, homologue, fragment or derivative thereof, incubated without the compound, thereby determining whether the compound stimulates or inhibits bioactive peptide proteolysis. In one embodiment of the method, the fragment may be from the N-terminal region of the human SEP and provides a method to identify allosteric modulators of the human SEP. In another embodiment of the present invention, the fragment may be from the carboxy terminal region of the human SEP and provides a method to identify inhibitors or selective inhibitors of bioactive peptide proteolysis.

The bioactive peptides may be full-length or fragments thereof and may be produced recombinantly or, preferably, synthetically. Preferably, said bioactive peptides are small synthetic peptides capable of being modulated (preferably cleaved by proteolysis/hydrolysis) by human SEP. More preferably, said synthetic peptides are labelled (preferably, fluorescently labelled, more preferably fluorescently labelled with intramolecularly quenchable fluorogenic dyes such as can be used in the FRET assays described herein).

Since human SEP may be involved in regulating bioactive peptide activity and/or in proteolysis of biologically inactive peptides into their active form, references to “bioactive peptide(s)” (and the like) hereinabove can also be taken to mean references to “biologically inactive peptide(s)” (and the like), with the context within which these references exist to be construed mutatis mutandis, as appropriate. For example, where the inhibition of human SEP activity might lead to increased levels of bioactive peptide(s), such inhibition might also, or instead, lead to increased levels of biologically inactive peptide(s), thereby leading to reduced levels of peptide(s) in their “active form”.

A human SEP polypeptide, its immunogenic fragments or oligopeptides thereof can be used for screening therapeutic compounds in any of a variety of drug screening techniques. The polypeptide employed in such a test may be free in solution, affixed to a solid support, borne on a cell surface, or located intracellularly. The abolition of activity or the formation of binding complexes between a human SEP polypeptide and the agent being tested may be measured.

Accordingly, the present invention relates to a method for screening one or a plurality of compounds for modulation (preferably specific modulation, such as specific binding affinity or inhibition) of human SEP or the expression thereof, or a portion thereof or variant, homologue, fragment or derivative thereof, comprising providing one or a plurality of compounds; combining a human SEP or a nucleotide sequence coding for the same or a portion thereof or variant, homologue, fragment or derivative thereof with the or each of a plurality of compounds for a time sufficient to allow modulation under suitable conditions; and detecting, for example, (i) binding of a human SEP, or portion thereof or variant, homologue, fragment or derivative thereof, to each of the plurality of compounds, thereby identifying the compound or compounds which modulate a human SEP or a nucleotide sequence coding for the same; or (ii) inhibition of a human SEP, or portion thereof or variant, homologue, fragment or derivative thereof, by each of the plurality of compounds, thereby identifying the compound or compounds which modulate (inhibit) a human SEP or a nucleotide sequence coding for the same. In such an assay, the plurality of compounds may be produced by combinatorial chemistry techniques known to those of skill in the art.

Another technique for drug screening provides for high throughput screening (HTS) of compounds having suitable binding affinity to the human SEP polypeptides and is based upon the method described in detail in Geysen, WO 84/03564, published on Sep. 13, 1984. In summary, large numbers of different small peptide test compounds are synthesised on a solid substrate, such as plastic pins or some other surface. The peptide test compounds are reacted with human SEP fragments and washed. A bound human SEP is then detected—such as by appropriately adapting methods well known in the art. A purified human SEP can also be coated directly onto plates for use in the aforementioned drug screening techniques. Alternatively, non-neutralising antibodies can be used to capture the peptide and immobilise it on a solid support.

This invention also contemplates the use of competitive drug screening assays in which neutralising antibodies capable of binding a human SEP specifically compete with a test compound for binding a human SEP. In this manner, the antibodies can be used to detect the presence of any peptide, which shares one or more antigenic determinants with a human SEP.

The assay method of the present invention may be a high throughput screen (HTS). In this regard, the teachings of WO 84/03564 may be adapted for the human SEP of the present invention.

The teachings of U.S. Pat. No. 5,738,985 may also be adapted for the assay method of the present invention.

Specifically contemplated by the present invention are FRET assays using, for instance, the labelled substrate peptide Rhodamine green-Gly-Gly-dPhe-Leu-Arg-Arg-Val-Cys(QSY™-7)-βAla-NH₂ (SEQ ID NO: 8), which is cleavable by the human SEP described above. In addition, FRET assays as described above, wherein the human SEP enzyme is replaced by any peptidase, are also contemplated by the present invention. Preferably, said peptidase is an exopeptidase or an endopeptidase. More preferably, said exopeptidase is oxytocinase and said endopeptidase is NEP or non-human SEP.

### AGENTS

The present invention also provides one or more agents identified by the assays, methods and identification methods of the present invention.

The agent of the present invention can be, for example, an organic compound or an inorganic compound. The agent can be, for example, a nucleotide sequence that is antisense to all or part of the sequence shown in SEQ ID NO: 1 or SEQ ID NO: 5. Preferably, the agent will be a modulator of SEP activity, more preferably a SEP inhibitor (SEPi) or a selective SEPi.

A SEPi is a compound which inhibits the enzymatic activity of SEP, that is prevents it cleaving (by proteolysis) a substrate peptide, polypeptide or protein.

The invention further provides an agent of the present invention (or even a pharmaceutically acceptable salt thereof, or a pharmaceutically acceptable solvate thereof) or a pharmaceutical composition containing any of the foregoing, for use as a medicament.

The present invention also relates to the use of an agent to affect human SEP activity (such as to inhibit, selectively inhibit, modulate or agonise) in any one or more of the urogenital system, cardiovascular system, the neurological system, and the endocrine system.

It is to be understood that any agent (which includes, but is not limited to, a modulator, inhibitor or selective inhibitor of human SEP) that is identified using any assay (or modification thereof) described above is also deemed an aspect of the present invention. Moreover, it is also to be understood that any agent (which includes, but is not limited to, modulators, inhibitors or selective inhibitors), which is capable of modulating (preferably inhibiting or selectively inhibiting) any peptidase and that is identified using any assay (or modification thereof) described above is also deemed an aspect of the present invention. Preferably, said agent (which includes, but is not limited to, modulators, inhibitors or selective inhibitors) modulates (preferably inhibits or selectively inhibits) an exopeptidase or an endopeptidase. More preferably, said exopeptidase is oxytocinase and said endopeptidase is NEP or non-human SEP.

### PHARMACEUTICALS

The present invention also provides a pharmaceutical composition for treating an individual in need of the same due to human SEP activity, the composition comprising a therapeutically effective amount of an agent that affects (such as inhibits or selectively inhibits) said activity and a pharmaceutically acceptable carrier, diluent or excipient.

Thus, the present invention also covers pharmaceutical compositions comprising the agents of the present invention (an agent capable of modulating the expression pattern of the nucleotide sequence of the present invention or the activity of the expression product thereof and/or an agent identified by an assay according to the present invention). In this regard, and in particular for human therapy, even though the agents of the present invention can be administered alone, they will generally be administered in admixture with a pharmaceutical carrier, excipient or diluent selected with regard to the intended route of administration and standard pharmaceutical practice.

By way of example, in the pharmaceutical compositions of the present invention, the agents of the present invention may be admixed with any suitable binder(s), lubricant(s), suspending agent(s), coating agent(s), or solubilising agent(s).

In general, a therapeutically effective daily oral or intravenous dose of the agents of the present invention is likely to range from 0.01 to 50 mg/kg body weight of the subject to be treated, preferably 0.01 to 20 mg/kg, more preferably 0.1 to 20 mg/kg. The agents of the present invention may also be administered by intravenous infusion, at a dose which is likely to range from 0.001-10 mg/kg/hr.

Thus, the present invention also provides a method of treating an individual in need of the same due to human SEP activity comprising administering to said individual an effective amount of a pharmaceutical composition comprising an agent identified by a method of the present invention.

Typically, the physician will determine the actual dosage which will be most suitable for an individual patient and it will vary with the age, weight, sex and response of the particular patient. The above dosages are exemplary of the average case. There can, of course, be individual instances where higher or lower dosage ranges are merited, and such are within the scope of this invention.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavemosally, intravenously, intramuscularly or subcutaneously. For parenteral administration, the compositions may be best used in the form of a sterile aqueous solution, which may contain other substances, for example enough salts or monosaccharides to make the solution isotonic with blood. For buccal or sublingual administration the compositions may be administered in the form of tablets or lozenges which can be formulated in a conventional manner.

For oral, parenteral, buccal and sublingual administration to subjects (such as patients), the daily dosage level of the agents of the present invention may typically be from 10 to 500 mg (in single or divided doses). Thus, and by way of example, tablets or capsules may contain from 5 to 100 mg of active agent for administration singly, or two or more at a time, as appropriate. It is also possible to administer the agents of the present invention in sustained release formulations.

In some applications, generally in humans, oral administration of the agents of the present invention is the preferred route, being the most convenient and can in some cases avoid disadvantages associated with other routes of administration—such as those associated with intracavernosal (i.c.) administration. In circumstances where the recipient suffers from a swallowing disorder or from impairment of drug absorption after oral administration, the drug may be administered parenterally.

For veterinary use, the agent of the present invention is typically administered as a suitably acceptable formulation in accordance with normal veterinary practice and the veterinary surgeon will determine the dosing regimen and route of administration which will be most appropriate for a particular animal. However, as with human treatments, it may be possible to administer the agent alone for veterinary treatments.

Typically, the pharmaceutical compositions—which may be for human or animal usage—will comprise any one or more of a pharmaceutically acceptable diluent, carrier or excipient. The choice of pharmaceutical carrier, excipient or diluent can be selected with regard to the intended route of administration and standard pharmaceutical practice. As indicated above, the pharmaceutical compositions may comprise as—or in addition to—the carrier, excipient or diluent any suitable binder(s), lubricant(s), suspending agent(s), coating agent(s) or solubilising agent(s).

The application yet further relates to pharmaceutical compositions which may comprise all or portions of human SEP polynucleotide sequences, human SEP antisense molecules, human SEP polypeptides, protein, peptide or organic modulators of human SEP bioactivity, such as inhibitors, selective inhibitors, antagonists (including antibodies), alone or in combination with at least one other agent, such as stabilising compound, and may be administered in any sterile, biocompatible pharmaceutical carrier, including, but not limited to, saline, buffered saline, dextrose, and water.

### GENERAL METHODOLOGY REFERENCES

Although in general the techniques mentioned herein are well known in the art, reference may be made in particular to Sambrook et al., Molecular Cloning, A Laboratory Manual (1989) and Ausubel et al., Short Protocols in Molecular Biology (1999) 4^{th} Ed, John Wiley & Sons, Inc. PCR is described in U.S. Pat. No. 4,683,195, U.S. Pat. No. 4,800,195 and U.S. Pat. No. 4,965,188.

### DEPOSIT

The following sample was deposited in accordance with the Budapest Treaty at the recognised depository The National Collections of Industrial, Food and Marine Bacteria (NCIMB) at 23 St. Machar Drive, Aberdeen, Scotland, AB24 3RY, United Kingdom on 29 Jun. 2001:

NCIMB number NCIMB 41110 is E. coli MSSE82.

The depositor was Pfizer Limited, Ramsgate Road, Sandwich, Kent, CT13 9NJ, United Kingdom.

One skilled in the art could readily grow the above-mentioned E. coli clone (NCIMB 41110) in Luria Broth containing ampicillin and isolate the plasmid DNA of the clone using the alkali lysis method as described in Sambrook, et al., eds. (1989) Molecular Cloning: A Laboratory Manual, Cold Spring Harbor Laboratory Press, New York, N.Y., USA. The di-deoxy termination method as described by Sanger et al. (Proceedings of the National Academy of Sciences (USA), (December 1977), 74(12):5463-5467) and modified by Applied Biosystems, Foster City, Calif., USA (see Applied Biosystems manufacturer's literature) for fluorescent detection could then be used to sequence the DNA and identify human SEP.

The terms “cell” or “cells” used herein when referring to the above-mentioned deposited biological material deposited under Accession Number NCIMB 41110 are interchangeable with the equivalent terms “micro-organism” or “micro-organisms” or “bacterium” or “bacteria”.

The present application also encompasses sequences derivable and/or expressible from that deposit and embodiments comprising the same. The present application also encompasses partial sequences derivable and/or expressible from that deposit and embodiments comprising the same, wherein those partial sequences code for active polypeptides (active enzymatic sites). The present application also encompasses proteins comprising sequences derivable and/or expressible from that deposit and embodiments comprising the same. The present application also encompasses proteins comprising partial sequences derivable and/or expressible from that deposit and embodiments comprising the same, wherein those partial sequences code for active polypeptides (active enzymatic sites).

### EXAMPLES

### Example 1

### Identification of Human SEP

Database Mining

The novel gene of human SEP was found by mining databases of human expressed sequence tags (ESTs) using the neprilysin protein as probe and the BLAST algorithm. EST hits (database=Incyte Gold™; gene id.=241161) were then assembled into contiguous sequence which predicted a large fragment of coding sequence. This corresponds to the catalytic domain by homology with the neprilysin protein sequence (60% identity). Probes for PCR cloning were designed using this region of the predicted coding sequence for the novel gene. Thereafter, further mining of unfinished sequence from the human genomic database identified 5 predicted exons, which were hypothesised to belong to the same novel gene in the missing N-terminal region. This was later confirmed following sequencing of the full length clone obtained from human testis library (see below).

The novel human SEP sequence includes 3′ UTR and part of the coding sequence in the highly conserved C-terminal region of human SEP which contains one of the two active catalytic sites. The novel human SEP appears to contain an insert of 37 amino-acids.

Isolation of Full Length Human SEP cDNA

An oligonucleotide (5′-ctgtcttgatggattggatg-3′) was designed using partial human SEP cDNA sequence from the above mentioned assembly of contiguous expression sequence tags (ESTs) that would enable longer human SEP cDNAs to be amplified from cDNA libraries using 5′-RACE (rapid amplification of 5′ cDNA ends) PCR.

A panel of 12 arrayed 96-well format human Rapid-Screen™ cDNA libraries were then screened by 5′-RACE PCR. cDNAs corresponding to human SEP were identified in libraries derived from brain, liver, placenta, small intestine, and testis.

None of these cDNAs were full length, so further 5′-RACE was performed on the panel of libraries using a primer (5′-gtccttggcagtcgaattctcc-3′) designed from the sequence of one of the longer, but partial length cDNAs from testis. This identified a longer (˜3.0 kb), putative full length cDNA clone in the testis library, which was isolated and sequenced from both ends. The full length human SEP clone was termed MSSE82 (and was deposited at NCIMB under Accession Number NCIMB 41110), with the full length cDNA cloned into the pCMV6-XL4 vector which has Genbank accession number #AF067196.

SEQ ID NO: 1 shows the nucleotide sequence (cDNA) coding for human SEP.

SEQ ID NO: 2 shows human SEP protein predicted from translation of cDNA sequence in +1 open reading frame.

SEQ ID NOS: 3 and 4 show oligonucleotide primer sequences used in the identification of the nucleotide sequence (cDNA) coding for human SEP.

SEQ ID NO: 5 shows the nucleotide sequence (cDNA) coding for human SEP—including 5′ and 3′ partial vector sequences (the first 65 nucleotides and last 17 nucleotides, respectively).

### Example 2

### Tissue Distribution of Human SEP mRNA

A multiple tissue messenger RNA blot was probed for human SEP. SEP mRNA was detected in a testis sample but not in other tissues.

A fragment of DNA corresponding to the entire human SEP coding sequence was amplified by PCR using the SEP clone MSSE82 as a template. The fragment was radiolabelled with ³²P dCTP using a megaprime kit (Amersham PLC, UK). The radiolabelled fragment was used as a hybridisation probe to screen a multiple tissue mRNA array (dot blot) (from Clontech, USA) containing mRNAs from a selection (76) of different human tissues and cell lines. Following hybridisation and washing, the blot was subjected to autoradiography. A signal was detected from the testis, but not other tissues.

RT-PCR analysis has shown human SEP mRNA also in the salivary gland and thyroid gland as well as testis (data not shown).

### Example 3

### Production of Recombinant SEP Enzyme

A culture of Chinese Hamster Ovary (CHO) cells is transfected with the plasmid MSSE82 using the lipofectamine method as described in the lipofectamine reagent protocol (Invitrogen Ltd, Paisley, UK). The cell media is harvested at 24 or 48 hours post-transfection, and cleared of cell debris by centrifugation at 3000 g for 5 min. The media is then dialysed overnight at 4° C. against 50 mM HEPES pH7.4/10% glycerol, using a “slide a lyser” (from Pierce and Warner, Chester UK). The dialyzed sample is then frozen in aliquots and stored under liquid nitrogen.

### Example 4

### Assays of Peptidase Activity

The utilisation of FRET to analyse the activity of peptidases is exemplified herein by homogeneous FRET assays for the analysis of the endopeptidases NEP (neutral endopeptidase) and SEP (soluble secreted endopeptidase).

Background

The novel homogeneous peptidase assays of the present invention are based on a FRET assay developed by Carvalho et al. for use with NEP (Carvalho et al., Annal. Biochem. 237, pp. 167-173 (1996)). The peptidase FRET assays utilise a similar, although not identical, intramolecularly quenched fluorogenic peptide substrate, but substituted with a novel combination of fluorogenic donor/acceptor dyes, such as Rhodamine green (Molecular Probes, Inc.) and QSY™7 (Molecular Probes, Inc.), or 5-(and 6) tetramethylrhodamine *mixed isomers* (Molecular Probes, Inc.) and QSY™7, or 5-carboxyfluorescein (Molecular Probes, Inc.) and 5-(and 6) tetramethylrhodamine *mixed isomers*.

The peptidase activity of, for example, the endopeptidases NEP or SEP is measured by monitoring their ability to proteolyse the synthetic peptide substrate Acceptor dye-Gly-Gly-dPhe-Leu-Arg-Arg-Val-Cys(donor dye)-βAla-NH₂ (SEQ ID NO: 8). For example, CP4 is cleaved by endopeptidases NEP or SEP, creating the cleavage product Rhodamine green-Gly-Gly-dPhe-Leu-Arg-Arg-OH (SEQ ID NO: 9), the release of which is monitored by fluorescence.

The two fluorophores (fluorogenic dyes) chosen for these assays have overlapping emission and absorption spectra and hence are suitable for energy transfer. The Rhodamine green acts as a donor and when excited at 485 nm gives out an emission (fluorescence) at 535 nm which in turn excites the QSY™7 (FRET is occurring). The QSY™7 is fluorescently silent and so gives off no emission above 535 nm hence no signal is observed (the Rhodamine green emission is quenched).

Upon cleavage (selective hydrolysis) by NEP or SEP at the Arg-Val peptide bond of the peptide substrate, the Rhodamine green and QSY™7 moieties move apart and so upon excitation at 485 nm, energy transfer can no longer take place. As a result, an increase in fluorescence is observed at 535 nm for the Rhodamine green. Other suitable fluorophores exhibit similar, but not identical, behavior and are excited and emit energy at different wavelengths depending on each dyes' individual properties.

Preparation of the Synthetic Peptide Substrates

Peptide assembly was completed on 0.25 mmol FMOC-PAL-PEG-PS resin by solid phase peptide synthesis protocols using modifications to manufacturer supplied (Applied Biosystems, Foster City, Calif., USA) 9-fluoreneylmethoxycarbonyl (FMOC)-based synthesis cycles. Our modified cycles deprotect the amino terminus with 2×5 minute treatments with 20% piperidine/N-methylpyrrolidinone (NMP); the efficiency of which is monitored by UV absorbance at 301 nm by passage of a small aliquot of deprotection solution through a UV absorbance detector. In a separate cartridge, the incoming amino acid is activated with 0.9 equivalents each of 2-(1H-Benzotriazole-1-yl)-1,1,3,3 tetramethyluronium hexafluorophosphate (HBTU)/1-Hydroxybenzotriazole (HOBt) dissolved in N,N-dimethylformamide (DMF). 2 equivalents of diiusopropylethyl amine (DIEA) are added. Concurrently, the resin is then washed with NMP to remove deprotection by-products. The wash solution is drained from the resin and the activated amino acid ester is transferred to the resin and stirred to allow coupling to the amino terminus for 20 minutes. The residual coupling solution is drained and the resin washed again with NMP. To ensure peptide homogeneity, a solution of 0.4M Acetic Anhydride/0.04M HOBt in NMP and 12 mmole DIEA are added to the resin to acetylate any potential unreacted sites. Finally, the resin is washed with NMP, drained, then washed with a mixture of 1:1 dichloromethane/2,2,2-trifluoroethanol and drained. This typifies one cycle of peptide synthesis. The completed synthesis resin was cleaved and deprotected using Reagent K (King, D. S. et. al., (1990), Int. J. Pep. Prot. Res., 36, pp. 255-66) affording 251 mg (100%) crude peptide CP1 Electrospray mass spectrometry (ESMS) (m/z calculation (calc.)=977.21 (MH+average), obs.=977.47).

Preparation of Rhodamine green-Gly-Gly-dPhe-Leu-Arg-Arg-Val-Cys(QS™7)-βAla-NH₂ (CP4)

50 mg (51 μmol) of crude CP1 was dissolved in solution of 10% DIEA/DMF containing 45 mg (52.4 μmol) QSY™-7 maleimide After 10 minutes, the reaction was judged to be incomplete via HPLC-MS analysis and an additional 30 mg (30.7 μmol) crude peptide was added. After 30 additional minutes, the reaction was judged via HPLC-MS to be complete and all starting reagents consumed. The product was isolated by C18 preparative HPLC chromatography and fractions exhibiting desired product molecular weight by Matrix Assisted Laser Desorption lonisation mass spectrometry (MALDI-MS) were pooled and lyophilised to 73.7 mg (50%) of a purple powder, CP2 ESMS (m/z calc.=1797.86 (MH+monoisotopic), obs.=1797.86).

73.7 mg (41 μmol) of CP2 was dissolved in a 2% DIEA/DMF solution containing 35 mg (52.8 μmol) Rhodamine Green carboxylic acid, trifluoroacetamide, succinimidyl ester (5(6)-CR 110 TFA, SE) *mixed isomers*. After 2 hours, the reaction was judged to be complete via HPLC-MS analysis. The product was isolated via C4 preparative HPLC chromatography and fractions exhibiting desired product molecular weights (MALDI-MS) were pooled and lyophilised to 71.4 mg (74%) of a purple powder CP3 ESMS (m/z calc.=2345.92 (MH+monoisotopic), obs.=2345.47).

71.4 mg (30.4 μmol) of CP3 was dissolved in 10 ml 4:1 CH₃CN/H₂O. To this was added 200 mg (1886 μmol) Na₂CO₃. After 16 hr. vortexing, the supernatant was decanted from the insoluble material. The reaction vessel was rinsed with 1 ml DMSO; this was combined with the supernatant and the product isolated via C4 preparative HPLC chromatography. Fractions exhibiting product molecular weights (MALDI-MS) were combined and lyophilised to 64 mg (98%) of a purple powder, CP4 ESMS (m/z calc.=2155.54 (MH+average), obs.=2155.27). CP4 is the desired synthetic peptide substrate Rhodamine green-Gly-Gly-dPhe-Leu-Arg-Arg-Val-Cys(QSY™7)-βAla-NH₂.

Preparation of 5-(and 6) tetramethyl rhodamine Gly-Gly-dPhe-Leu-Arg-Arg-Val-Cys(QSY™7)-βAla-NH₂ (CP5)

CP5 was prepared in an analogous manner to CP4 starting with identically prepared CP2. 1.05 mg (0.58 μmol) of CP2 was dissolved in 360 ul 2% DIEA/DMF. To this was added 30 μl of a 10 mg/ml (0.432 μmol) solution of 5-(and-6)-carboxytetramethylrhodamine, succinimidyl ester (5(6)-TAMRA, SE) *mixed isomers*. After 4 hours, the reaction was judged to be complete via HPLC-MS analysis. The product was isolated via C4 preparative chromatography and fraction exhibiting the desired product molecular weights (MALDI-MS) were pooled and lypohilyzed to 1.35 mg (99+%) CP5 ESMS (m/z calc.=2323.81(MH+average), obs.=2323.42)

Preparation of 5-carboxyfluorescein-Gly-Gly-dPhe-Leu-Arg-Arg-Val-Cys(5-(and 6)tetramethylrhodamine)-βAla-NH₂ (CP6)

First, 5-carboxyfluorescein-Gly-Gly-dPhe-Leu-Arg-Arg-Val-Cys(H)-βAla-NH₂ (CP7) was prepared. 512 mg (0.137 mmol/gm, therefore 0.07 mmol) of the synthesis resin elaborated to afford CP1 was slurried in 5 ml DMF containing 40 mg (0.085 mmol) 5-carboxyfluorescein, succinimidyl ester (5-FAM, SE) *single isomer*. After 1 hour, the resin was filtered, washed with 3×15 ml DMF followed by washes with 3×15 ml dichloromethane and dried in vacuo affording 300 mg tagged resin. The peptide CP7 was cleaved from the solid support with Reagent K (above) yielding 50 mg (54%) CP7, ESMS (m/z calc.=1335.51(MH+average), obs.=1335.02).

10 mg (7.5 μmol) CP7 was dissolved in 1 ml DMF. To this was added a solution of 5 mg tetramethylrhodamine-5-maleimide *single isomer* dissolved in 1 ml 20% DIEA/DMF. After 90 minutes, the reaction was judged to be complete via HPLC-MS analysis. The product was isolated via C4 preparative chromatography and the fractions exhibiting the desired product molecular weights (MALDI-MS) were pooled and lyophilyzed affording 1.15 mg (8%)CP6 ESMS (m/z calc.=1817.02 (MH+average), obs.=1816.91)

Materials

All reagents were purchased of the highest commercial purity available and were used without further refinement. All reagents for peptide synthesis were purchased from Applied Biosystems, Foster City, Calif., USA with the following exceptions: QSY™-7 maleimide (Catalogue number Q-10257), Rhodamine Green carboxylic acid, trifluoroacetamide, succinimidyl ester (5(6)-CR 110 TFA, SE) *mixed isomers* (Catalogue number R-6112) , 5-(and-6)-carboxytetramethylrhodamine, succinimidyl ester (5(6)-TAMRA, SE) *mixed isomers* (Catalog number C1171), 5-carboxyfluorescein, succinimidyl ester (5-FAM, SE) *single isomer*(Catalog number C2210) and tetramethylrhodamine-5-maleimide *single isomer* (Catalog number T-6027) were all purchased from Molecular Probes, Inc., Oregon, USA; FMOC-PAL-PEG-PS was purchased from Perceptive Biosystems, Massachusetts, USA (Catalogue number GEN913384); FMOC-B-Alanine and FMOC-d-phenylalanine were purchased from Novabiochem, California, USA; FMOC-Arg(Pbf)-OH was purchased from AnaSpec, Inc., California, USA; 2,2,2-Trifluoroethanol was purchased from Aldrich, Wis., USA. Sodium Carbonate was purchased from Fisher, Pa., USA.

Preparative HPLC chromatography was performed on Vydac (California, USA) C18 (Catalogue number 218TP1022) or C4 (Catalogue number 214TP1022) columns at 10 ml/min flow rate eluting with a linear gradient of 0% to 80%(A=5% CH₃CN/0.1% TFA/94.9% H₂O, B=100% CH₃CN) over 30 minutes collecting 30 second time fractions. Analytical HPLC-MS was performed using a Micromass (Manchester, UK) LCT mass spectrometer (masses based on externally calibrated standards) coupled with a Waters (Massachusetts, USA) 2690 HPLC inlet and a Waters 996 photodiode array detector performing chromatography on a Vydac C4 (Catalogue number 214TP5415) column with a linear gradient of 0% to 80%(A=5% CH₃CN/0.1% TFA/94.9% H₂O, B=100% CH₃CN) over 30 minutes at 1 ml/min flow rate. Deconvoluted molecular weights were calculated from multiply charged observed ions using Micromass transform software. MALDI-MS were obtained on a Perceptive Biosystems Voyager-DE linear mass spectrometer using alpha cyano 4-hydroxy cinnamic acid matrix (Hewlett Packard, California, USA) and reported masses based on external calibration.

Process (Including Chemical Structures)

CP4 (=synthetic peptide substrate Rhodamine green-Gly-Gly-dPhe-Leu-Arg-Arg-Val-Cys(QSY™-7)-βAla-NH₂) is synthesised by incorporating the key intermediate CP3 in a solid phase peptide synthesis scheme.

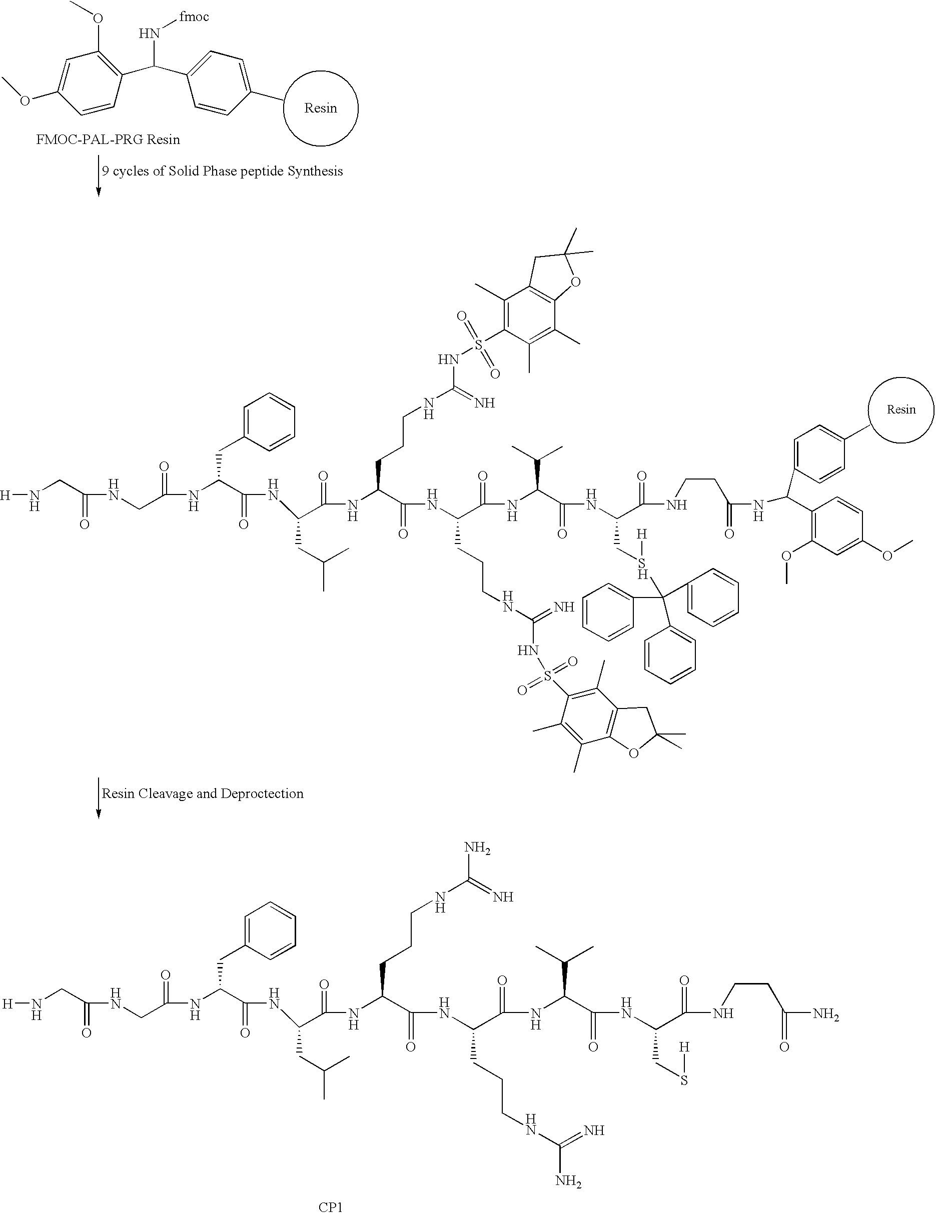

In summary, FMOC-PAL-PEG Resin is elaborated using Solid Phase Peptide Synthesis protocols optimised for efficiency of yield and time. These cycles (full details supra) incorporate 2 FMOC deprotections, washes, a single coupling of HBTU activated amino acid, washes, capping and finally, washing first with NMP then with 1:1 trifluoroethanol/dichloromethane. These washes help to relax resin secondary structure allowing for thorough deprotection and efficient coupling of the next incoming amino acid during the next cycle.

CP2 is synthesised (full details supra) as follows:

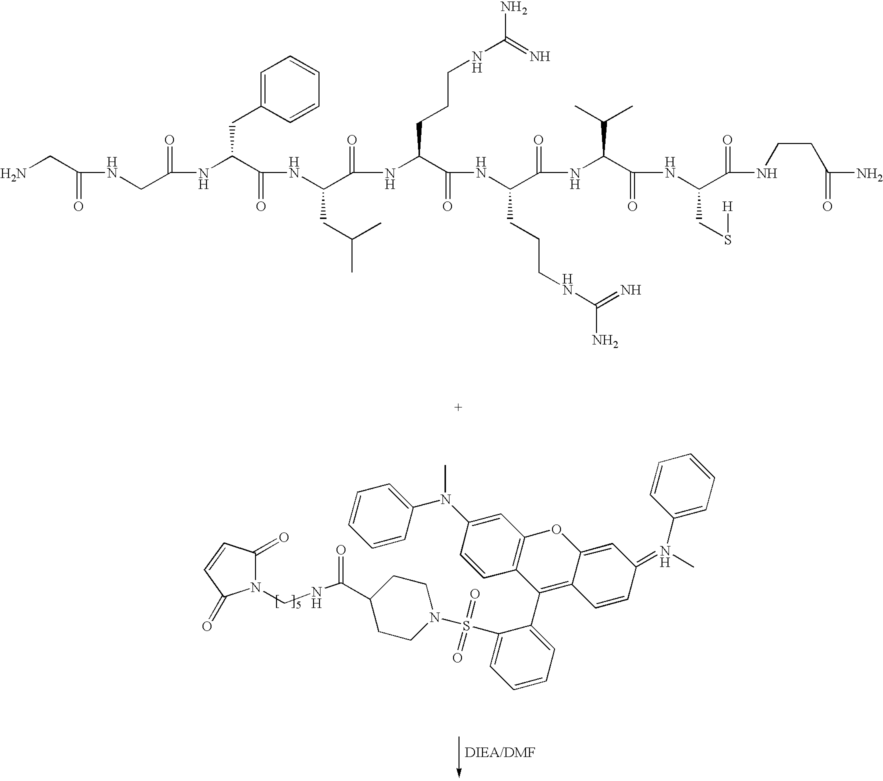

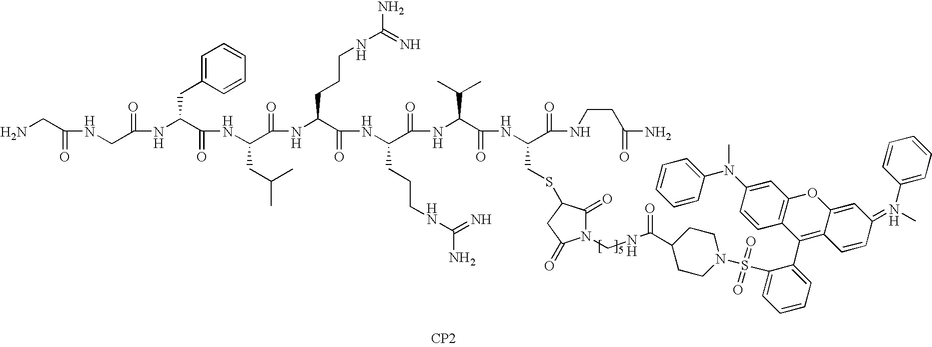

Following this incorporation of the QSY™-7 tag, the second fluorophore, Rhodamine Green is added as the bis-trifluoroacetyl protected dye as shown below.

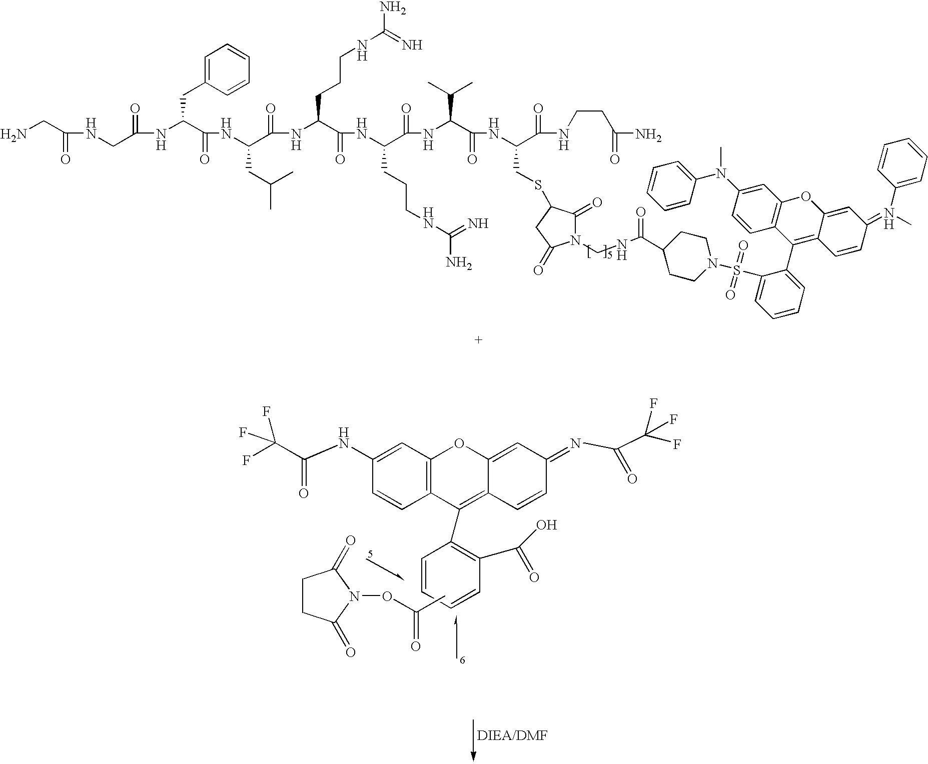

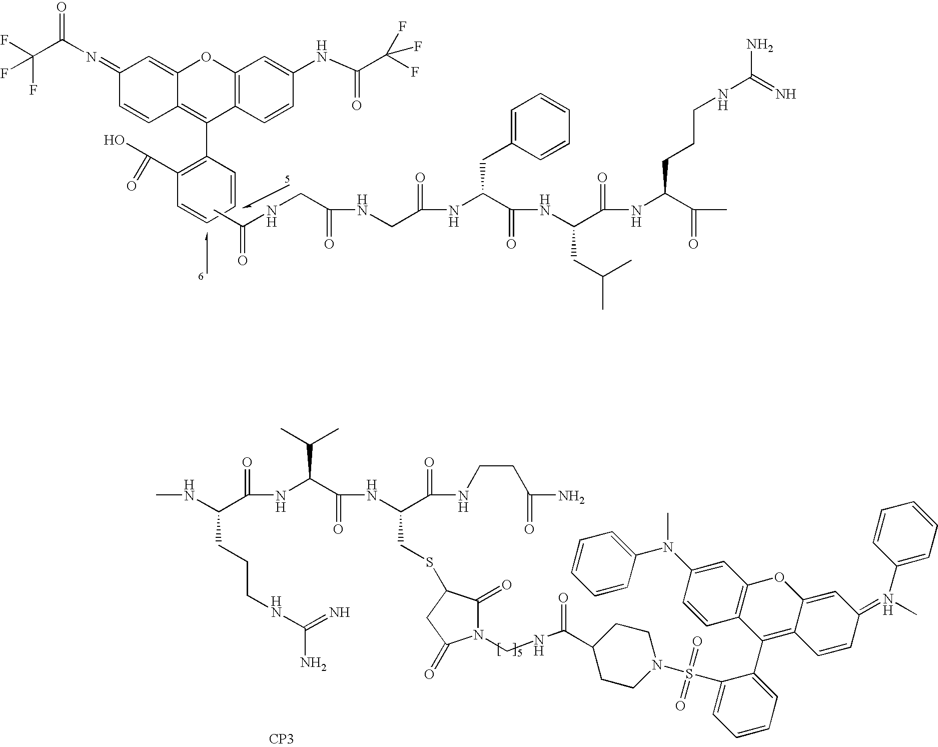

Finally, the trifluoroacetyl groups are removed by treatment with Na₂CO₃ affording the desired substrate, CP4.

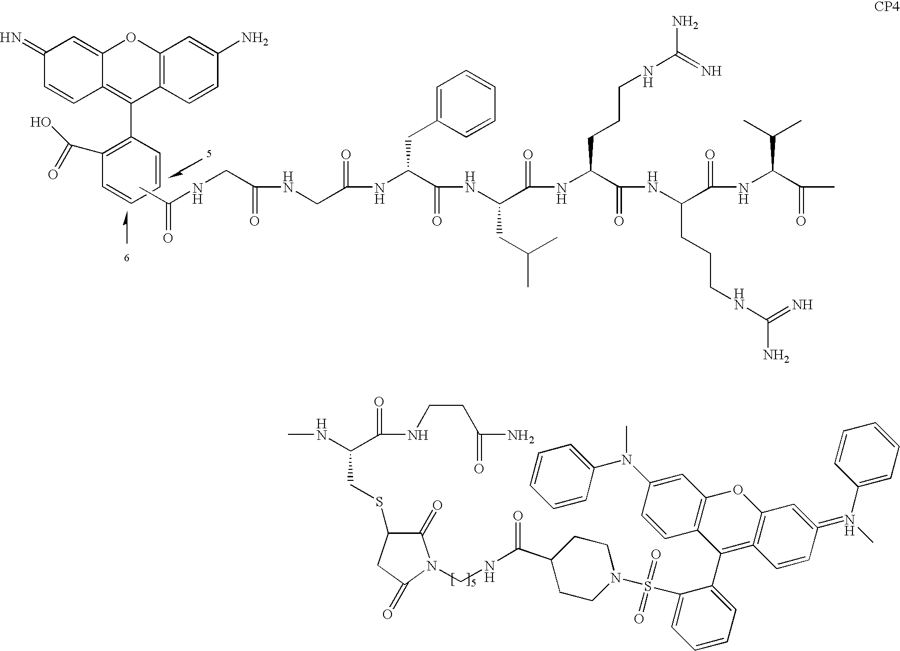

CP5 is prepared analogously to CP4 where 5-carboxyfluorescein, succinimidyl ester (5-FAM, SE) *single isomer* is substituted in place of Rhodarnine Green carboxylic acid, trifluoroacetamide, succinimidyl ester and, without necessity of the trifluoroacetyl protecting groups, affords directly CP5 from the coupling reaction:

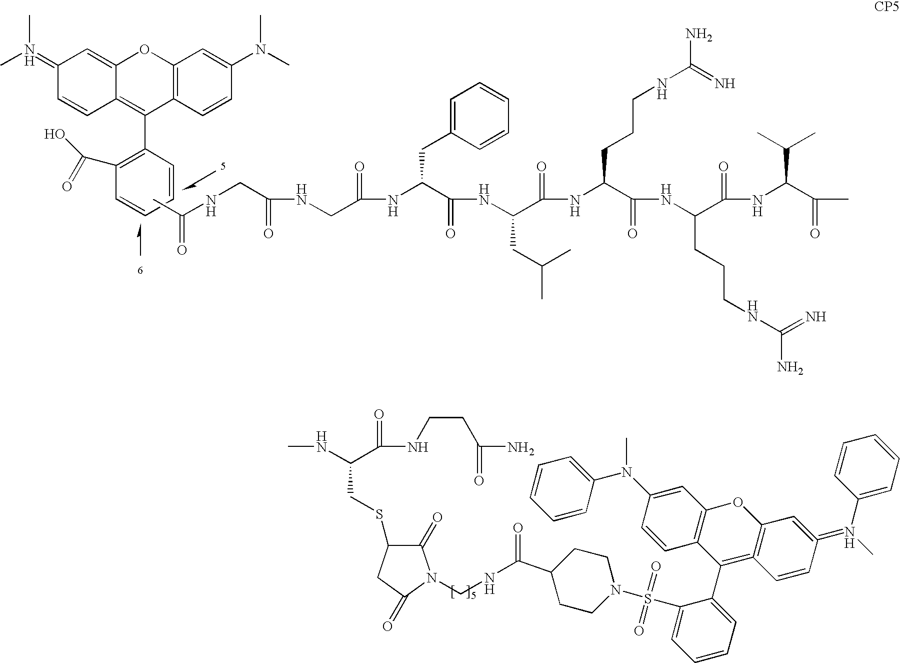

CP6 is prepared according to the flow diagram below:

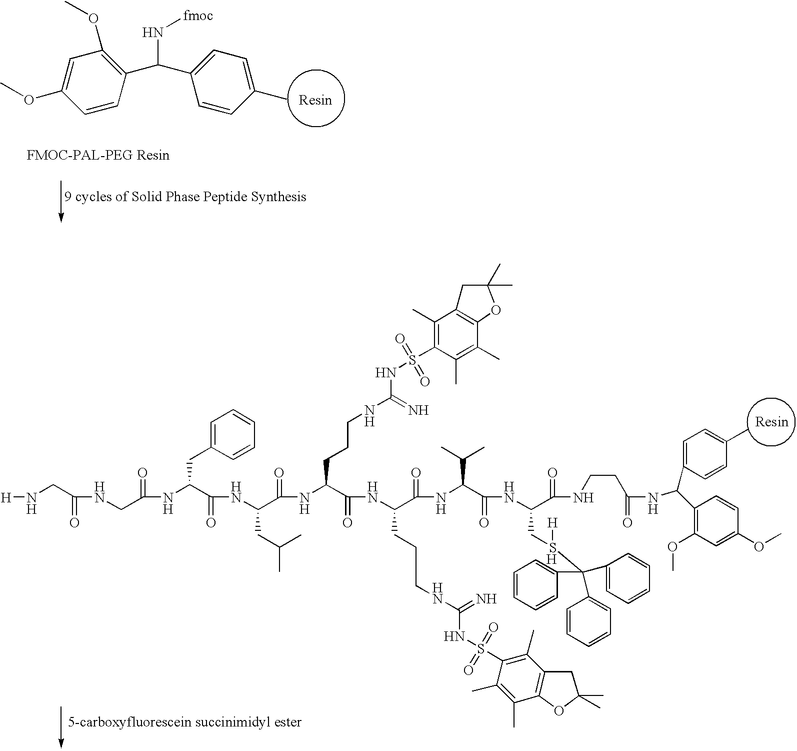

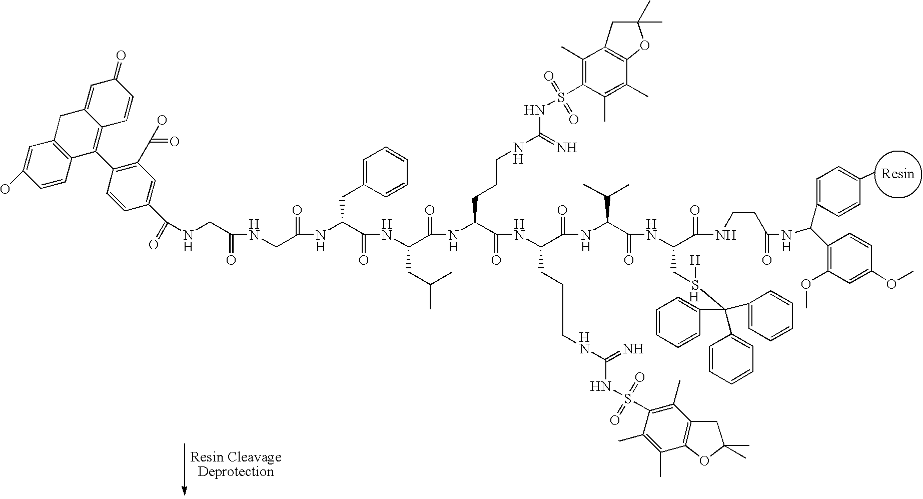

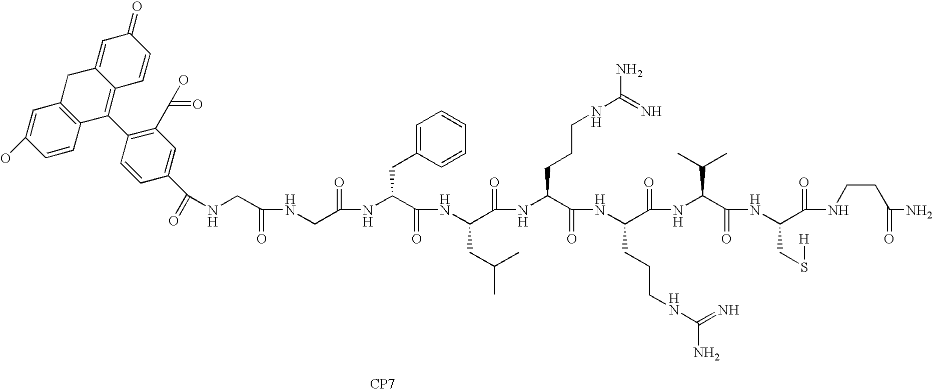

CP7 is then tagged with 5-tetramethylrhodamine maleimide affording CP6

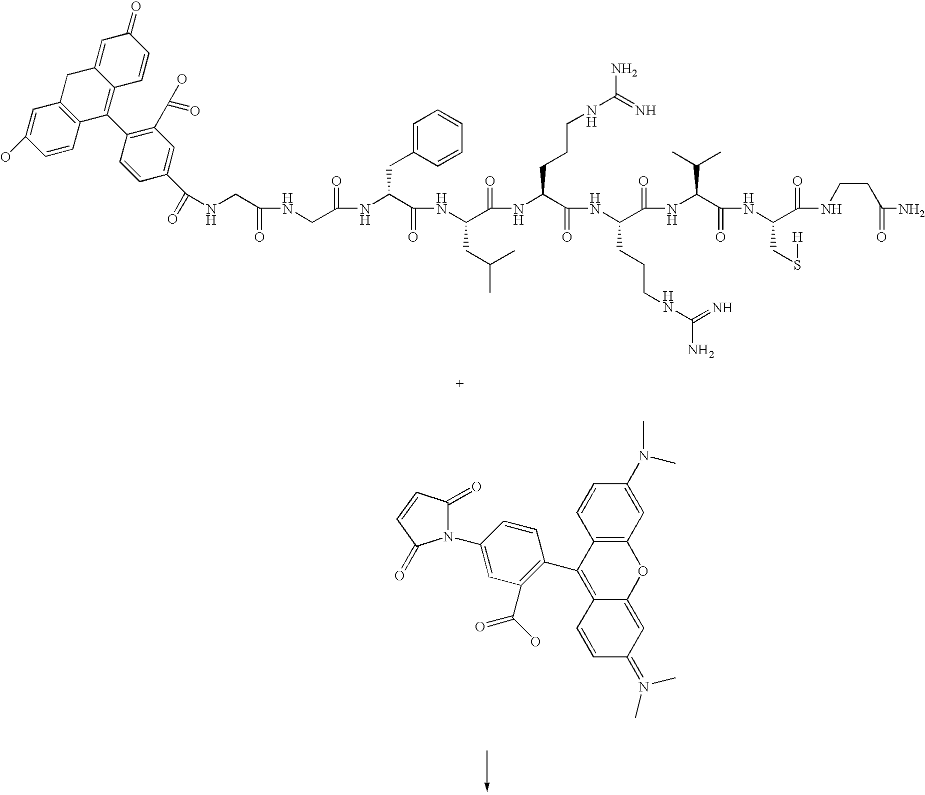

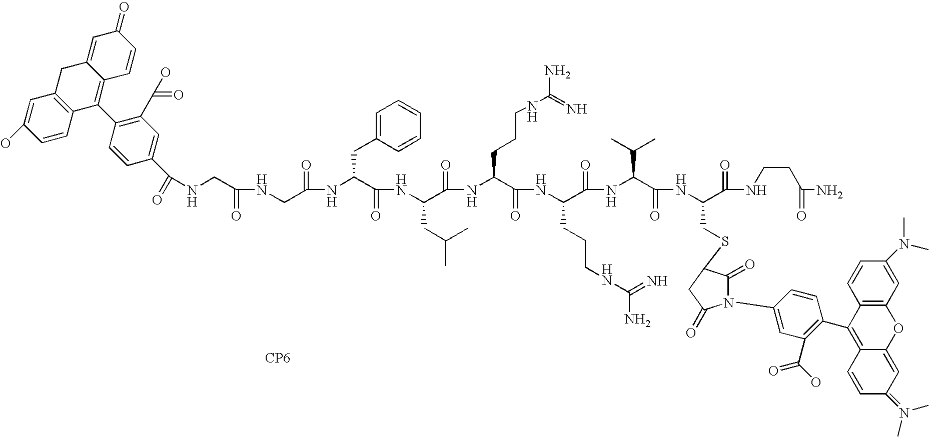

FRET Assays

(a) NEP FRET Assay

Introduction

The following Example describes the development and screening of a novel homogeneous FRET-based assay, for a metalloendopeptidase, specifically NEP. This 384 well assay utilises a novel substrate (the preparation of which is described above) with a low kₘ value, which makes it cost effective for high throughput screening (HTS). In addition the fluorophores used in design of the substrate have desirable optical properties to avoid compound interference.

Following assay development, the assay was successfully transferred and validated on a Robolab 9600 linear track screening robot (Robocon, Vienna, Austria). Examples of the high quality of this assay are given below along with the speed at which the HTS was run, achieving an average throughput of 62,000 data points within a 24 hour period.

Materials and Methods

All assays were conducted in 384 well black, flat bottom plates, purchased from Matrix Technologies Ltd. (Cheshire, UK).

Compounds for screening were supplied as singles at 40 μM in 1% DMSO to achieve a final screening concentration of 10 μM.

The peptide substrate (Rhodamine green-Gly-Gly-dPhe-Leu-Arg-Arg-Val-Cys(QSY™7)-βAla-NH₂) (SEQ ID NO: 8) was made as described above and labelled with fluorophores purchased from Molecular Probes, Inc. All other reagents were purchased from Sigma/Aldrich (Poole, Dorset, UK).

Recombinant human NEP enzyme was expressed using Pichia pastoris and purified to homogeneity using a three-step purification procedure.

All assays were performed in 50 mM Hepes buffer pH 8, 0.1% v/v pluronic-F127 and 1% v/v protease inhibitor cocktail (antipain, aprotinin, leupeptin, pepstatin A). 10 μl of compound in 1% DMSO was added to each well, followed by 20 μl of recombinant enzyme (10 nM final concentration). The reaction was initiated by the addition of 10 μl of substrate (1 μM final concentration) and plates were incubated at 25° C. for one hour. Termination of the reaction was achieved by the addition of 20 μl of Stop (excess of a known inhibitor) to a concentration of 10 μM in the well. Plates were counted using Tecan Ultra (485 nm excitation, 535 nm emission; Tecan Austria, Salzburg, Austria).

Assay Development and Steady State Kinetics

The steady state kinetics of the NEP enzyme were investigated in terms of k_{cat} and kₘ values (see Table 1), which were in good agreement with the literature. In addition, NEP enzyme concentration and reaction time were optimised and a suitable stopping reagent identified (an excess of a potent standard). Solvent tolerance was also investigated and found to be acceptable up to 1% DMSO final concentration in the assay. Finally the assay had an absolute requirement for 0.1% pluronic F127, this is thought to be due to the peptide substrate being very hydrophobic and sticking to the wells in the 384 well plate.

TABLE 1

| Kinetic Parameter | Experimental Value | Standard (Std) Error |
|---|---|---|
| Kₘ | 8 μM | 0.3 |
| K_{cat} | 5 min₋₁ | |
| K_{cat}/Kₘ | 10,500 M⁻¹ S⁻¹ | |

HTS Assay Validation and Screening

The assay was successfully transferred on to a Robolab 9600 screening robot (Robocon) using two standard validation procedures:

1. Reagent stability was measured over time by calculation of the Assay Value Ratio (AVR); and

2. A subset of the Pfizer compound collection was screened to determine the percentage cut-off for selection of primary active compounds (actives).

Reagent stability is measured over time to determine whether 24 hour screening is possible on the automated system. AVR or Assay Value Ratio is a statistical measure of assay quality which is calculated using the formula shown below. Acceptable statistics are obtained when AVR≦0.6 (z′≧0.4).

AVR=((3*STD_{highs}+3*STD_{lows})/(AVE_{highs}−AVE_{lows}))

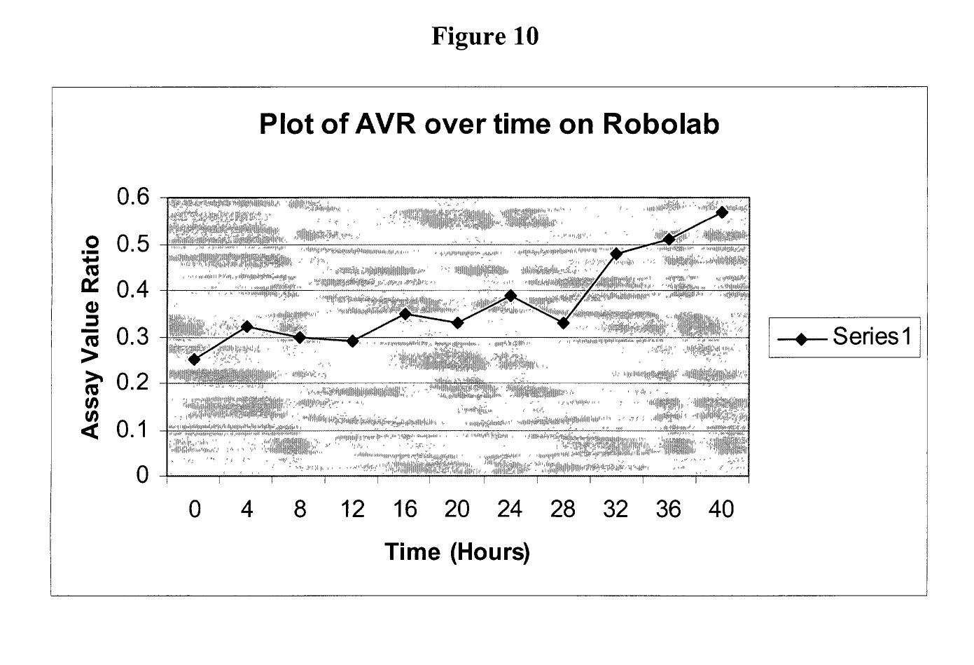

In order to measure AVR values, screening plates were prepared with maximum (0.25% DMSO final concentration) and minimum (excess of a potent standard) wells. Each AVR plate was then scheduled on the robot using a four hour cycle time. The results are illustrated in FIG. 10.

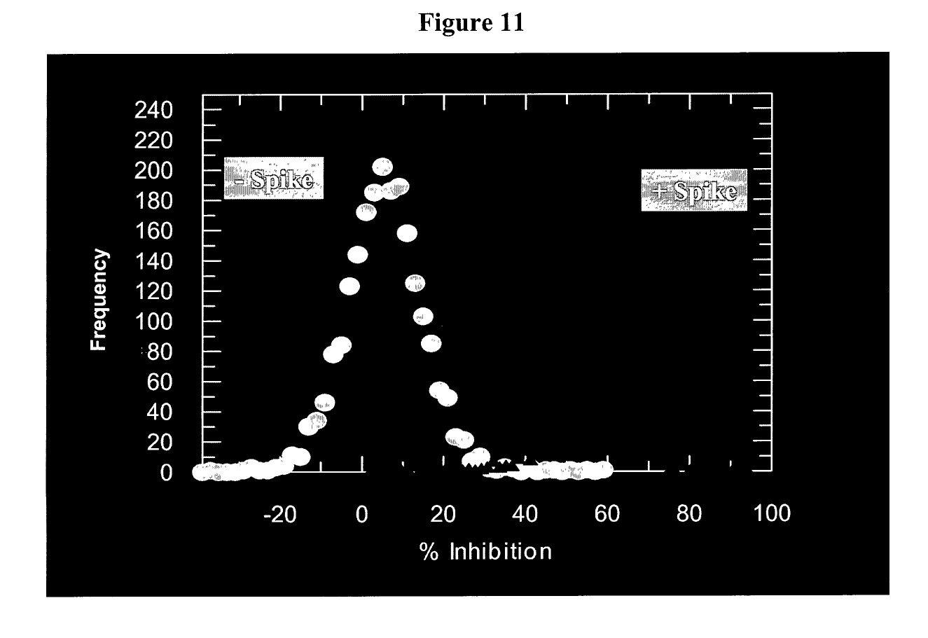

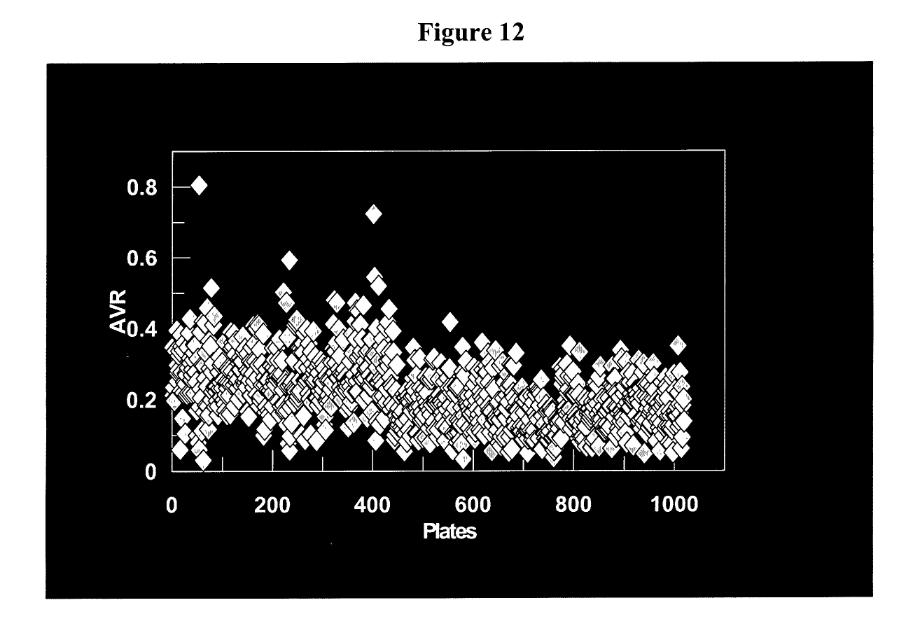

The second part of the validation process, involved screening a subset of the Pfizer compound collection to determine the percentage cut-off for selection of primary active compounds. Two sets of identical compound plates were screened, one set spiked with a known concentration of inhibitor and the other minus spike. The results are shown in FIG. 11.

Primary screening was completed in seven scheduled robot runs, achieving an average throughput of 170 plates (62,000 compounds) per day. Plates were scheduled on the robot using an eight minute cycle time. Control and enzyme were added to plates using a Tecan Genesis liquid handling robot (Tecan AG, Hombrechtikon, Switzerland). Substrate was added using a Labsystems 384 multidrop (Labsystems Oy, Helsinki, Finland) and stop solution was added using a Robocon Reag liquid dispenser (Robocon). All reagents were chilled to 4° C.

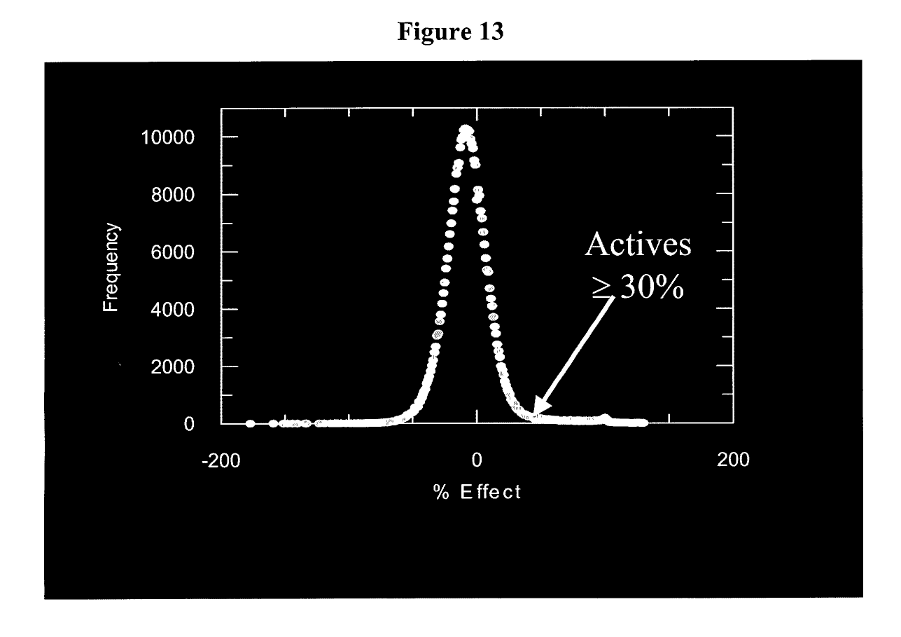

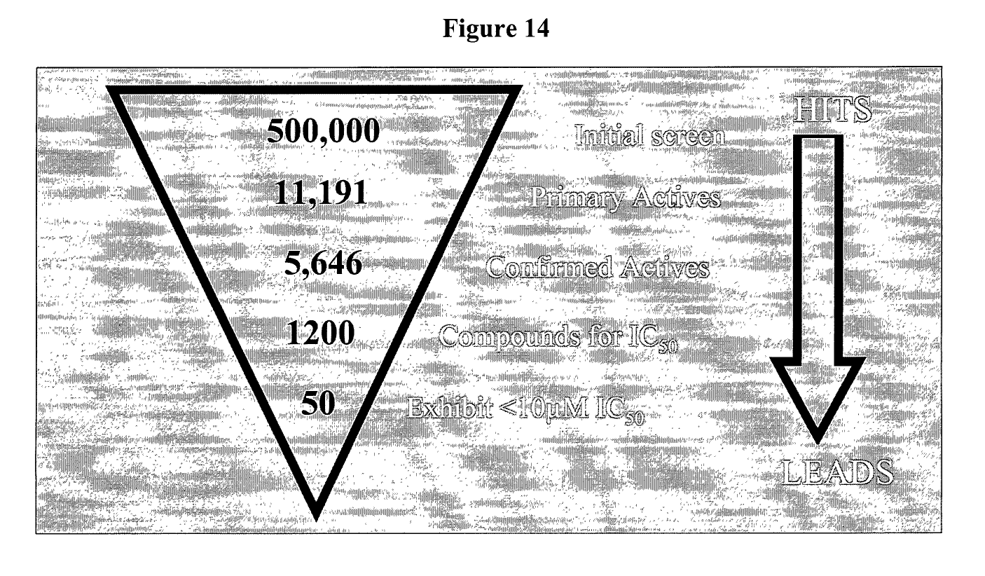

FIGS. 13 and 14 illustrate the high assay quality achieved throughout the HTS. AVR values were consistently of a high standard and actually improved during screening.

All actives from the primary screening campaign were ordered from the Pfizer Automated Liquid Sample Bank (ALSB) and re-tested on a fully integrated Tecan Genesis, Genmate, Ultra system (Tecan Austria; Tecan AG). Fifty percent of the primary actives confirmed upon retest and 1200 compounds were selected for IC₅₀ determination.

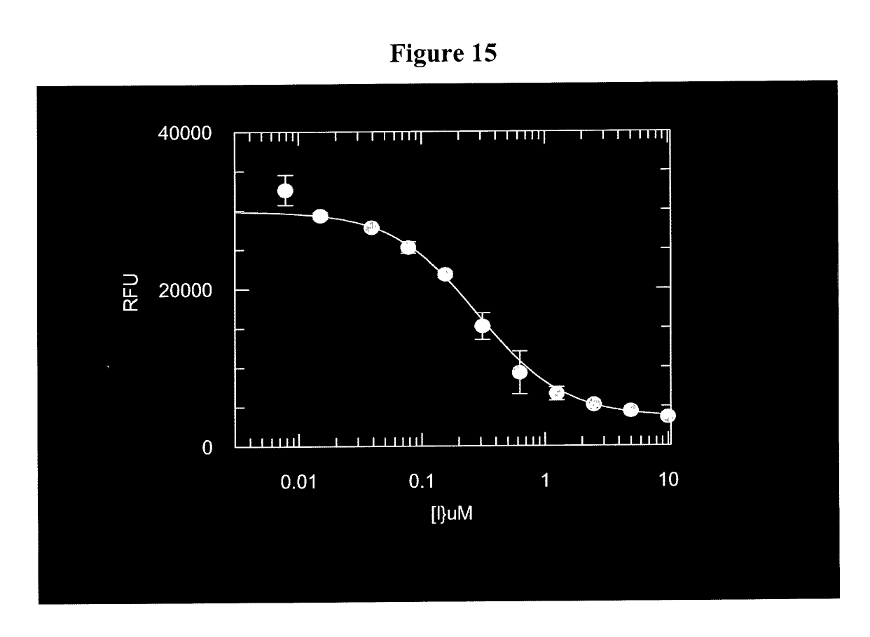

The quality of the IC₅₀ data was of a high standard and from the 1200 confirmed actives, three hits under 1 μM were identified. An example of the data obtained from one of these compounds is shown in FIG. 14. All IC₅₀ curves (an example is shown in FIG. 15) were fitted to a full four parameter logistic equation using either Grafit 4 (Erithacus Software, Horley, Surrey, UK) or the Pfizer HTS data handling programme.

Assay Miniaturisation

Following successful completion of the 384 well HTS, assay miniaturisation into low volume 384 Coming/Costar plates (Coming Costar UK, High Wycombe, Buckinghamshire, UK) and 1536 well Greiner plates (Greiner Labortechnik Ltd., Gloucestershire, UK) was investigated. The data from these experiments are shown below in Table 2.

TABLE 2

| Assay Format | Total Assay Volume | AVR |
|---|---|---|
| 96 well | 80 μl | 0.2 |
| 384 well | 40 μl | 0.2 |
| Low volume 384 well | 11 μl | 0.45 |
| 1536 well | 5.5 μl | 0.70 |

Summary and Conclusions

Novel NEP inhibitors (NEPi) have been identified using a novel 384 well FRET-based assay. The assay was developed following the definition of the steady state kinetics of the NEP enzyme and successfully validated in 384 well fonnat for high throughput screening (HTS). The HTS campaign was carried out using a Robocon 9600 linear track screening robot (Robocon). An average throughput of 62,000 data points per day was achieved and the primary HTS was completed in seven scheduled runs. This facilitated the identification of a range of diverse inhibitors, several of which exhibited sub-micromolar potency.

Following the 384 well HTS, we investigated assay miniaturisation in low volume 384 and 1536 well plates. The initial results were very encouraging and further work is planned to optimise the assay. A low volume 384 well assay would be a useful model for evaluating new 384 and 1536 liquid handling technologies.

In summary the above provides an Example of a versatile, homogenous fluorescence based assay for HTS, amenable to both automation and miniaturisation. As compound numbers continue to grow and the need for increases in throughput and reduction in cost become the key drivers for HTS, it is expected that fluorescence based screens will continue to play an ever increasing role in the future.

(b) SEP FRET Assay

The SEP FRET assay follows essentially the same processes as set out above for the NEP FRET assay. However, there are a few differences in view of the enzyme under investigation.

Reagents for the Assay are First Prepared as Follows:

A substrate solution is made up by resuspending the substrate Rhodamine green-Gly-Gly-dPhe-Leu-Arg-Arg-Val-CyS(QSY™7)-βAla-NH₂ (SEQ ID NO: 8) in 50 mM HEPES buffer pH7.4 (Sigma, UK) at a concentration of 2 μM, then adding 1 EDTA-free protease inhibitor cocktail tablet (Roche Diagnostics, UK) per 25 ml.

An aliquot of SEP enzyme described above is thawed then diluted in 50 mM HEPES, pH7.4 by a predetermined factor specific to each enzyme batch, such that 50 μl contains sufficient enzyme to convert approximately 30% of substrate to product during the assay.

A 4% DMSO solution comprised of 4 ml DMSO plus 96 ml 50 mM HEPES pH7.4 is prepared.

A product solution is prepared by adding 500 μl of substrate solution to 250 μl enzyme solution plus 250 μl of 4% DMSO solution, and incubating at 37° C. for 16 hours.

Assays Are Set Up As Follows:

In a black 96 well microtitre plate, 100 μl of substrate solution is added to 50 μl of 4% DMSO solution. A similar non-specific background blank is also set up in which the 50 μl of 4% DMSO solution additionally contains 40 μM phosphoramidon. 50 μl of enzyme solution is added to the assay and blank, and the 96 well plate placed in a BMG galaxy fluorescence reader, operating with the Biolise software package (BMG Lab technologies, Offenberg, Germany).

The plate is incubated in the fluorescence reader for 1 hour at 37° C. and a fluorescence measurement taken every 3 minutes (Excitation (Ex) 485 nm/Emission (Em) 535 nm). The proteolytic activity of SEP corresponds to the rate of increase in fluorescence of the sample—rate of increase in fluorescence units of the non-specific background blank. The maximum velocity measurement (MaxV) calculated by the software over four successive readings is used for this calculation.

A fluorescence measurement taken from 200 μl of product in a well on an identical microtitre plate is taken. If required, this value is used, together with the measured fluorescence units from the 60 min timepoint of the SEP assay, to calculate the percentage (%) of the substrate proteolysed during the 1 hour incubation period or to convert the measured rates of fluorescence increase into other useful units such as ng substrate proteolysed/min/ml enzyme.

The assay is used to calculate enzyme kinetic parameters such as Vmax and Km following standard principles described in Fundamentals of Enzyme Kinetics by Athel Cornish Bowden, 1979, published by Butterworths.

Using the SEP FRET Assay to Determine The Inhibition Parameters of SEP Inhibitors

To determine the IC₅₀ of SEP inhibitors (SEPi; for example, phosphoramidon), multiple SEP assays are performed as described above with a range of test concentrations of inhibitor included in the 50 μl of DMSO solution (made by appropriate dilution of a 10 mM 100% DMSO stock of inhibitor with 4% DMSO/50 mM HEPES pH7.4.). Using a suitable standard graph fitting computer program, a sigmoidal dose response curve is fitted to a plot of log inhibitor concentration versus MaxV (or % inhibition or % activity). The IC₅₀ is calculated as the inhibitor concentration causing 50% maximal inhibition. Typically for a given IC₅₀ determination, a dose range of at least 10 inhibitor concentrations differing in half log unit increments is used.

The SEP assay is used to determine the Ki and mode of inhibition (i.e. whether the inhibition is competitive, mixed, non-competitive, etc.) following standard enzymology principles as described, for example, in Fundamentals of Enzyme Kinetics by Athel Cornish Bowden, 1979, published by Butterworths.

The above SEP FRET assay has also been adapted to use the 384 well plate format (as per the NEP FRET assay hereinbefore described). As per the NEP FRET assay, 0.1% v/v pluronic-F127 was also required for efficient functioning of the smaller well formatted assay.

### Example 5

### An Animal Model of Sexual Arousal

In our studies we have developed a robust reproducible model of the physiology of male and female sexual arousal. This model uses an anaesthetised rabbit and employs Laser Doppler technologies to monitor intracavernosal pressure and genital blood flow whilst routinely recording cardiovascular parameters. We are capable of measuring small changes in intracavemosal pressure within the penis and vaginal (and even clitoral) blood flow induced by pelvic nerve stimulation or infusion of VIP in the absence and presence of test agents.

We believe that our animal model directly reflects the clinical data. Hence, this model can be used to study candidate agents for the prophylaxis and/or treatment of MED and FSAD, such as measuring enhancement of penile erection via increases in intracavemosal pressure and enhancement of vaginal or clitoral blood flow.

Identifying VIP and Other Neuropeptides as Potential Substrates That Are Involved In Male and Female Sexual Arousal

The ability of SEP to degrade the pro-sexual neuropeptide known as the vasoactive intestinal peptide (VIP) is measured by making use of, for example, a radioimmunoassay (RIA), such as the VIP RIA, that can be obtained commercially from Peninsula Laboratories, California, USA.

A sample of SEP enzyme, typically 5-100 μl of recombinant SEP (produced by the method described above) is incubated with a sample of VIP peptide, typically 1-10 ng for 5 hours at 37° C., in a buffer such as 50 mM HEPES, pH7.4. A negative control identical to this but also containing 10 μM phosphoramidon is set up and treated in an identical manner. After the incubation period, the quantity of VIP remaining in both the sample and the negative control is determined using the RIA as per manufacturer's instructions. The reduction in quantity of VIP in the sample relative to the negative control is a measure of the VIP proteolysing activity of SEP. The assay is used to determine enzyme kinetic parameters such as Vmax and Km following standard principles as described, for example, in Fundamentals of Enzyme Kinetics by Athel Cornish Bowden, 1979, published by Butterworths.

### Example 6

### Animal Test Methods for SEP Inhibitors

Female Sexual Dysfunction Animal Model

Anaesthetic Protocol

Female New Zealand rabbits (˜2.5 kg) are pre-medicated with a combination of Medetomidine (Domitor®) 0.5 ml/kg i.m., and Ketamine (Vetalar®) 0.25 ml/kg i.m. whilst maintaining oxygen intake via a face mask. The rabbits are tracheotomised using a Portex™ uncuffed endotracheal tube 3 ID., connected to a ventilator and maintained at a ventilation rate of 30-40 breaths per minute, with an approximate tidal volume of 18-20 ml, and a maximum airway pressure of 10 cm H₂O. Anaesthesia is then switched to Isoflurane and ventilation continued with O₂ at 2 l/min. The right marginal ear vein is cannulated using a 23G or 24G catheter, and Lactated Ringer solution perfused at 0.5 ml/min. The rabbit is maintained at 3% Isoflurane during invasive surgery, dropping to 2% for maintenance anaesthesia.

Cannulation of Vessels

The left groin area of the rabbit is shaved and a vertical incision is made approximately 5cm in length along the thigh. The femoral vein is exposed, isolated and then cannulated with a PVC catheter (17G; Portex Limited, Hythe, Kent, UK) for the infusion of drugs and compounds. Cannulation is repeated for the femoral artery, inserting the catheter to a depth of 10 cm to ensure that the catheter reaches the abdominal aorta. This arterial catheter is linked to a Gould system to record blood pressure. Samples for blood gas analysis are also taken via the arterial catheter. Systolic and diastolic pressures are measured, and the mean arterial pressure calculated using the formula [(diastolic ×2+systolic)÷3]. Heart rate is measured via the pulse oxymeter and Po-ne-mah data acquisition software system (Ponemah Physiology Platform, Gould Instrument Systems, Inc., Ohio, USA).

Stimulation of the Pelvic Nerve

A ventral midline incision is made into the abdominal cavity. The incision is about 5 cm in length just above the pubis. The fat and muscle is bluntly dissected away to reveal the hypogastric nerve which runs down the body cavity. It is essential to keep close to the side curve of the pubis wall in order to avoid damaging the femoral vein and artery, which lie above the pubis. The sciatic and pelvic nerves lie deeper and are located after further dissection on the dorsal side of the rabbit. Once the sciatic nerve is identified, the pelvic nerve is easily located. The term pelvic nerve is loosely applied; anatomy books on the subject fail to identify the nerves in sufficient detail. However, stimulation of the nerve causes an increase in vaginal and clitoral blood flow, and innervation of the pelvic region. The pelvic nerve is freed away from surrounding tissue and a Harvard bipolar stimulating electrode is placed around the nerve. The nerve is slightly lifted to give some tension, then the electrode is secured in position. Approximately lml of light paraffin oil is placed around the nerve and electrode. This acts as a protective lubricant to the nerve and prevents blood contamination of the electrode. The electrode is connected to a Grass S88 Stimulator. The pelvic nerve is stimulated using the following parameters: 5V, pulse width 0.5 ms, duration of stimulus 10 seconds and a frequency range of 2 to 16 Hz. Reproducible responses are obtained when the nerve is stimulated every 15-20 minutes.

A frequency response curve is determined at the start of each experiment in order to determine the optimum frequency to use as a sub-maximal response, normally 4 Hz. The compound(s) to be tested are infused, via the femoral vein, using a Harvard 22 infusion pump allowing a continuous 15 minute stimulation cycle.

Positioning of the Laser Doppler Probes

A ventral midline incision is made, at the caudal end of the pubis, to expose the pubic area. Connective tissue is removed to expose the tunica of the clitoris, ensuring that the wall is free from small blood vessels. The external vaginal wall is also exposed by removing any connective tissue. One Laser Doppler flow probe is inserted 3 cm into the vagina, so that half the probe shaft is still visible. A second probe is positioned so that it lay just above the external clitoral wall. The position of these probes is then adjusted until a signal is obtained. A second probe is placed just above the surface of a blood vessel on the external vaginal wall. Both probes are clamped in position.

Vaginal and clitoral blood flow is recorded either as numbers directly from the Flowmeter using Po-ne-mah data acquisition software (Ponemah Physiology Platform, Gould Instrument Systems, Inc.), or indirectly from Gould chart recorder trace. Calibration is set at the beginning of the experiment (0-125 ml/min/100 g tissue).

Infusion of Vasoactive Intestinal Peptide (VIP)

The doses of VIP (Bachem, H-3775) infused are 2.0, 6.0, 20.0, 60.0 μg/kg i.v. and are infused in a volume of 0.5 ml of saline. VIP is infused using a Harvard 22 pump, infusing at 500 μl/min via a 3-way tap into the femoral vein. After VIP infusion, the catheter is flushed with heparinised saline (Hepsaline) so that no VIP is left in the catheter.

For experiments using VIP infusions, there is a need for an initial sensitising dose response curve (2-60 μg/kg), in order that reproducible responses are obtained. An initial infusion of Hepsaline (50UI/ml) is infused to act as a negative control.

Infusion of Inhibitors

SEP inhibitors and vehicle controls are infused at the same rate as VIP. SEP inhibitors are left for 30 minutes prior to a VIP dose response curve, and left for 15 minutes prior to pelvic nerve stimulation.

Data are expressed as mean genital (vagina/clitoris) blood flow ±standard error of the mean (s.e.m.). Significant changes are identified using Student's t-tests.

Male Sexual Dysfunction Animal Model

Anaesthetised Rabbit Methodology

Male New Zealand rabbits (˜2.5 kg) are pre-medicated with a combination of Medetomidine (Domitor®) 0.5 ml/kg i.m., and Ketamine (Vetalar®) 0.25 ml/kg i.m. whilst maintaining oxygen intake via a face mask. The rabbits are tracheotomised using a Portex™ uncuffed endotracheal tube 3 ID., connected to a ventilator and maintained at a ventilation rate of 30-40 breaths per minute, with an approximate tidal volume of 18-20 ml, and a maximum airway pressure of 10 cm H₂O. Anaesthesia is then switched to Isoflurane and ventilation continued with O₂ at 2 l/min. The right marginal ear vein is cannulated using a 23G or 24G catheter, and Lactated Ringer solution perfused at 0.5 ml/min. The rabbit is maintained at 3% Isoflurane during invasive surgery, dropping to 2% for maintenance anaesthesia.

Cannulation of Vessels

The left groin area of the rabbit is shaved and a vertical incision is made approximately 5 cm in length along the thigh. The femoral vein is exposed, isolated and then cannulated with a PVC catheter (17G; Portex Limited) for the infusion of drugs and compounds. Alternatively, or in addition, the left jugular vein is exposed, isolated and then cannulated with a PVC catheter (17G; Portex Limited) for the infusion of drugs and compounds. Cannulation is repeated for the femoral artery, inserting the catheter to a depth of 10 cm to ensure that the catheter reaches the abdominal aorta. This arterial catheter is linked to a Gould system to record blood pressure. Samples for blood gas analysis are also taken via the arterial catheter. Systolic and diastolic pressures are measured, and the mean arterial pressure calculated using the formula [(diastolic ×2+systolic)÷3]. Heart rate is measured via the pulse oxymeter and Po-ne-mah data acquisition software system (Ponemah Physiology Platform, Gould Instrument Systems, Inc.).

Stimulation of the Pelvic Nerve

A ventral midline incision is made into the abdominal cavity. The incision is about 5 cm in length just above the pubis. The fat and muscle is bluntly dissected away to reveal the hypogastric nerve which runs down the body cavity. It is essential to keep close to the side curve of the pubis wall in order to avoid damaging the femoral vein and artery, which lie above the pubis. The sciatic and pelvic nerves lie deeper and are located after further dissection on the dorsal side of the rabbit. Once the sciatic nerve is identified, the pelvic nerve is easily located. The term pelvic nerve is loosely applied; anatomy books on the subject fail to identify the nerves in sufficient detail. However, stimulation of the nerve causes an increase in intracavernosal pressure and cavernosal blood flow, and innervation of the pelvic region. The pelvic nerve is freed away from surrounding tissue and a Harvard bipolar stimulating electrode is placed around the nerve. The nerve is slightly lifted to give some tension, then the electrode is secured in position. Approximately lml of light paraffin oil is placed around the nerve and electrode. This acts as a protective lubricant to the nerve and prevents blood contamination of the electrode. The electrode is connected to a Grass S88 Stimulator.

The pelvic nerve is stimulated using the following parameters: 5V, pulse width 0.5 ms, duration of stimulus 20 seconds with a frequency of 16 Hz. Reproducible responses are obtained when the nerve is stimulated every 15-20 minutes.

Several stimulations using the above parameters are performed to establish a mean control response. The compound(s) to be tested are infused, via the jugular vein, using a Harvard 22 infusion pump allowing a continuous 15 minute stimulation cycle. The skin and connective tissue around the penis is removed to expose the penis. A catheter set (Insyte-W, Becton-Dickinson 20 Gauge 1.1×48 mm, Beckton-Dickinson) is inserted through the tunica albica into the left corpus cavemosal space and the needle removed, leaving a flexible catheter. This catheter is linked via a pressure transducer (Ohmeda 5299-04) to a Gould system to record intracavemosal pressure. Once an intracavemosal pressure is established, the catheter is sealed in place using Vetbond (tissue adhesive, 3M). Heart rate is measured via the pulse oxymeter and Po-ne-mah data acquisition software system (Ponemah Physiology Platform, Gould Instrument Systems, Inc.).

Intracavernosal blood flow is recorded either as numbers directly from the Flowmeter using Po-ne-mah data acquisition software (Ponemah Physiology Platform, Gould Instrument Systems, Inc.), or indirectly from Gould chart recorder trace. Calibration is set at the beginning of the experiment (0-125 ml/min/100 g tissue).

Infusion of Inhibitors

The SEP inhibitor and vehicle controls are infused at a rate of 0.1 ml/second. SEP inhibitors are left for 15 minutes prior to pelvic nerve stimulation.

Data are expressed as mean intracavemosal blood pressure ± s.e.m. Significant changes are identified using Student's t-tests.

It will be appreciated that the foregoing is provided by way of example only and modification of detail may be made without departing from the scope of the invention.

For the avoidance of doubt, all references disclosed herein are incorporated by reference.

[image]

[image]

[image]

[image]

[image]

[image]

1. A method for identifying an agent which binds to and/or modulates a SEP polypeptide comprising contacting said agent with a polypeptide comprising: SEQ ID NO: 2, an amino acid sequence encoded by the polynucleotide sequence of SEQ ID NO: 1 or SEQ ID NO: 5; or a polypeptide encoded by the cDNA of NCIMB 41110, and determining whether binding and/or modulation occurs.

2. The method of claim 1, comprising contacting said SEP polypeptide with a SEP substrate peptide in the presence of said agent, wherein said substrate peptide is capable of providing a detectable signal in response to cleavage by said SEP polypeptide, wherein said agent is identified as a modulator of said SEP polypeptide if there is a difference in the detectable signal in the presence and in the absence of said agent.

3. The method of claim 2, wherein said method identifies an agent that decreases said detectable signal and is a SEP polypeptide inhibitor.

4. The method of claim 2 or claim 3, wherein said substrate peptide is labelled with at least one fluorescent donor dye and said signal is detected by Fluorescence Resonance Energy Transfer (FRET) assay.

5. The method of claim 4, wherein said labelled substrate peptide is Rhodamine green-Gly-Gly-dPhe-Leu-Arg-Arg-Val-CyS(QSY™7)-βAla-NH₂, 5-(and 6) tetramethyl rhodamine Gly-Gly-dPhe-Leu-Arg-Arg-Val-CyS(QSY™7)-βAla-NH₂ or 5-carboxyfluorescein-Gly-Gly-dPhe-Leu-Arg-Arg-Val-Cys(5-(and 6)tetramethylrhodamine)-βAla-NH₂.

6. The method of any one of claims 1 to 5, wherein binding between said SEP polypeptide and said agent is detected.

7. The method of claim 6, wherein said method is a competitive binding assay.

8. A method for identifying an agent which inhibits or selectively inhibits a peptidase comprising contacting said peptidase and a peptidase substrate peptide selected from the group consisting of Rhodamine green-Gly-Gly-dPhe-Leu-Arg-Arg-Val-Cys(QSY™7)-βAla-NH₂, 5-(and 6) tetramethyl rhodamine Gly-Gly-dPhe-Leu-Arg-Arg-Val-CyS(QSY™7)-βAla-NH₂ and 5-carboxyfluorescein-Gly-Gly-dPhe-Leu-Arg-Arg-Val-Cys (5- (and 6) tetramethylrhodamine)-βAla-NH₂, in the presence of said agent, wherein said substrate peptide is capable of providing a detectable signal in response to cleavage by said peptidase as detected by Fluorescence Resonance Energy Transfer (FRET) assay, wherein said agent is identified as an inhibitor of said peptidase if there is a decrease in the detectable signal in the presence of said agent as compared to in the absence of said agent.

9. The method of claim 8, wherein said peptidase is an exopeptidase or an endopeptidase.

10. The method of claim 8, wherein said peptidase is oxytocinase, neutral endopeptidase (NEP), or non-human SEP.

11. An agent identified by the method of any one of claims 1 to 10; wherein said agent is not phosphoramidon, thiorphan, fasidotrilat, omapatrilat or FR901533.

12. An agent identified by the method of any one of claims 1 to 7; wherein said agent has a greater than 30-fold selectivity for SEP over neutral endopeptidase NEP EC 3.4.24.11 and angiotensin converting enzyme (ACE).

13. An agent according to claim 12; wherein said agent has a greater than 50-fold selectivity for SEP over neutral endopeptidase NEP EC 3.4.24.11 and angiotensin converting enzyme (ACE).

14. An agent identified by the method of any one of claims 1 to 7; wherein said agent has a greater than 100-fold selectivity for SEP over endothelin converting enzyme (ECE).
